(19) United States
(12) Patent Application Publication
(10) Pub. No.: US 2026/0028410 A1
(43) Pub. Date: Jan. 29, 2026

(54) TREATMENT OF GRAFT VERSUS HOST DISEASES USING ANTI-CD122 ANTIBODY

(71) Applicant: FORTE SUBSIDIARY, INC., Dallas, TX (US)

(72) Inventor: Paul A. WAGNER, San Juan, PR (US)

(21) Appl. No.: 19/355,733

(22) Filed: Oct. 10, 2025

Related U.S. Application Data
(63) Continuation of application No. PCT/US2024/023897, filed on Apr. 10, 2024.
(60) Provisional application No. 63/495,533, filed on Apr. 11, 2023.

(51) Int. Cl. C07K 16/28; A61K 39/00; A61K 45/06; A61P 37/06

(52) U.S. Cl. CPC C07K 16/2866; A61K 45/06; A61P 37/06; A61K 2039/505; A61K 2039/54; C07K 2317/24; C07K 2317/565; C07K 2317/76

(57) ABSTRACT

Provided herein, in certain aspects, are anti-CD122 antibodies for use in methods for treatment of graft versus host diseases. In some embodiments, the anti-CD122 antibodies are used to treating acute graft versus host disease. In some embodiments, the anti-CD122 antibodies are used to treating chronic graft versus host disease. In some embodiments, the anti-CD122 antibodies are used in a combination therapy with a JAK inhibitor in methods for treatment of graft versus host diseases.

### CROSS-REFERENCE TO RELATED APPLICATIONS

This application is a continuation of International Patent Application No. PCT/US2024/023897 filed Apr. 10, 2024, which claims the benefit of U.S. provisional application Ser. No. 63/495,533 filed Apr. 11, 2023, which are hereby incorporated by reference in their entireties.

### INCORPORATION BY REFERENCE OF SEQUENCE LISTING

The present application is being filed along with a Sequence Listing in electronic format. The Sequence Listing is provided as a file entitled 53654-723.301_SL.XML, created Oct. 10, 2025, which is 264,449 bytes in size. The information in the electronic format of the Sequence Listing is incorporated by reference in its entirety.

### TECHNICAL FIELD

Described herein are methods of preventing and/or treating acute graft versus host disease and chronic graft versus host disease using an anti-CD122 antibody.

### BACKGROUND

Interleukin receptors are involved in mediating many cellular responses including T cell immune responses. The interleukin 2 receptor is present in three forms with respect to ability to bind interleukin 2 (IL2). The low affinity form of the receptor is a monomer of the interleukin 2 receptor subunit alpha (Gene Symbol: IL2RA; also known as CD25) and is not directly involved in signal transduction. The intermediate affinity receptor form is composed of a beta/gamma subunit heterodimer, while the high affinity receptor form is composed of an alpha/beta/gamma subunit heterotrimer. Both the intermediate and high affinity forms of the receptor are involved in receptor-mediated endocytosis and transduction pathways for IL2. The IL2RB gene encodes interleukin 2 receptor subunit beta (also known as CD122). The protein encoded by the IL2RB gene (CD122) is a type I transmembrane protein with its amino-(N-)terminal domains extracellular to the plasma membrane in mature forms. CD122 protein is primarily expressed in the hematopoietic system. The IL2RG gene encodes interleukin 2 receptor subunit gamma protein (also known as CD132) which serves as the gamma subunit of IL2 receptors. Interleukin 2 receptor beta/gamma subunit heterodimers (IL2Rβ/IL2Rγ complex) are composed of CD122/CD132. Interleukin 2 receptor alpha/beta/gamma subunit heterotrimers (IL2Ru/IL2Rβ/IL2Rγ complex) are composed of CD25/CD122/CD132.

In addition to functioning in IL2-mediating signaling as the interleukin 2 receptor subunit beta, CD122 also transmits signals from the cytokine interleukin 15 (IL15). Unlike the alpha subunit of the IL2 receptor, the interleukin 15 receptor subunit alpha (Gene Symbol: IL15RA; also known as CD215) is capable of binding its ligand (IL15) with high affinity independent of the other receptor subunits. IL15 signaling through trans-presentation of IL15 bound to the interleukin 15 receptor subunit alpha occurs to transmit signals through neighboring cells. IL15/IL15Ra bound complexes from a cell can initiate signal transduction in the trans-presentation conformation by interacting with IL15 beta/gamma receptors on neighboring cells. Interleukin 15 beta/gamma receptor heterodimers (IL15Rβ/IL15Rγ complex) have an intermediate affinity for IL15 and are composed of CD122/CD132. CD215 can also be found as part of interleukin 15 alpha/beta/gamma heterotrimers in a cis configuration for signal transduction. This IL15 receptor alpha/beta/gamma subunit heterotrimer (IL15Rα/IL15Rβ/IL15Rγ complex) is a high affinity IL15 receptor composed of CD215/CD122/CD132 and has a binding affinity for IL15 similar to that of CD215 monomer for IL15. It is through these various interleukin receptor complexes that CD122 is involved in transmitting signals from the cytokines IL2 and IL15.

A graft versus host disease can occur after an immune-competent graft is administered to a subject and graft versus host diseases are characterized by pathogenic inflammation in organs of affected subjects. Effective prophylactics and treatments for graft versus host diseases represent significant unmet medical burdens and there is a need for improved methods of preventing and/or treating graft versus host diseases.

### SUMMARY

Cancers originating in the blood and bone marrow have the potential for long term curative treatment through hematopoietic stem cell transplantation (HSCT). HSCT can replenish immune cells and may induce anticancer beneficial effects. However, in some cases, the success of this treatment is limited by the development of graft versus host disease (GvHD). A large proportion of patients undergoing allogeneic hematopoietic stem cell transplantation (alloHSCT) go on to develop acute or chronic GvHD. Either form of GvHD is triggered by the reactivity of donor-derived immune cells against allogenic tissues in the host and remains a major unmet medical need currently with limited treatment options. Acute and/or chronic GvHD symptoms and complications often markedly affect quality of life following alloHSCT and, along with cancer reoccurrence, progression of acute or chronic GvHD is a main cause of death after alloHSCT. In addition, acute and/or chronic GvHD is responsible for approximately 15-30% of complication-related deaths following HSCT. However, the therapeutic effectiveness of alloHSCT in treating malignancies is based on the allorecognition of donor T cells, which can induce a cytotoxic effect on tumor cells from the host. This is termed a graft versus leukemia (GvL) or a graft versus tumor (GvT) effect. While allorecognition of GvL or GvT provides a therapeutic benefit to subjects in need thereof, the same phenomenon of allorecognition of non-malignant cells, tissues, and organs of the host forms the basis of GvHD. A therapy aimed at strong immunosuppression to prevent or treat acute and/or chronic GvHD has potential to abrogate the beneficial GvL or GvT effect. Conversely, it is possible that weak or no immunosuppression increases a risk or severity of acute and/or chronic GvHD following alloHSCT.

Described herein, in some aspects, are methods of preventing or treating acute or chronic graft versus host disease (aGvHD or cGvHD) in a subject in need thereof. In an aspect, the methods comprise administering to the subject an effective amount of an anti-CD122 antibody, or its antigen-binding fragment thereof, comprising i) a variable heavy chain (VH) domain and ii) a variable light chain (VL) domain, wherein the VH domain comprises an HCDR1 sequence comprising a sequence selected from SEQ ID NOs: 1-11, an HCDR2 sequence comprising a sequence selected from SEQ ID NOs: 12-23, and an HCDR3 sequence comprising a sequence selected from SEQ ID NOs: 24-36, and the VL domain comprises an LCDR1 sequence comprising a sequence selected from SEQ ID NOs: 37-47, an LCDR2 sequence comprising a sequence selected from GTS, TTS, YTS, WAS, KAS, GAT, YAS or STS, and an LCDR3 sequence comprising a sequence selected from SEQ ID NOs: 56-67. In some embodiments, the HCDR1 sequence comprises SEQ ID NO: 7, the HCDR2 sequence comprises SEQ ID NO: 18, the HCDR3 sequence comprises SEQ ID NO: 30, the LCDR1 sequence comprises SEQ ID NO: 43, the LCDR2 sequence comprises YTS, and the LCDR3 sequence comprises SEQ ID NO: 62. In some embodiments, the HCDR1 sequence comprises SEQ ID NO: 1, the HCDR2 sequence comprises SEQ ID NO: 12, the HCDR3 sequence comprises SEQ ID NO: 24, the LCDR1 sequence comprises SEQ ID NO: 37, the LCDR2 sequence comprises GTS, and the LCDR3 sequence comprises SEQ ID NO: 56. In some embodiments, the HCDR1 sequence comprises SEQ ID NO: 2, the HCDR2 sequence comprises SEQ ID NO: 13, the HCDR3 sequence comprises SEQ ID NO: 25, the LCDR1 sequence comprises SEQ ID NO: 38, the LCDR2 sequence comprises TTS, and the LCDR3 sequence comprises SEQ ID NO: 57. In some embodiments, the HCDR1 sequence comprises SEQ ID NO: 3, the HCDR2 sequence comprises SEQ ID NO: 14, the HCDR3 sequence comprises SEQ ID NO: 26, the LCDR1 sequence comprises SEQ ID NO: 39, the LCDR2 sequence comprises YTS, and the LCDR3 sequence comprises SEQ ID NO: 58. In some embodiments, the HCDR1 sequence comprises SEQ ID NO: 4, the HCDR2 sequence comprises SEQ ID NO: 15, the HCDR3 sequence comprises SEQ ID NO: 27, the LCDR1 sequence comprises SEQ ID NO: 40, the LCDR2 sequence comprises WAS, and the LCDR3 sequence comprises SEQ ID NO: 59. In some embodiments, the HCDR1 sequence comprises SEQ ID NO: 5, the HCDR2 sequence comprises SEQ ID NO: 16, the HCDR3 sequence comprises SEQ ID NO: 28, the LCDR1 sequence comprises SEQ ID NO: 41, the LCDR2 sequence comprises YTS, and the LCDR3 sequence comprises SEQ ID NO: 60. In some embodiments, the HCDR1 sequence comprises SEQ ID NO: 6, the HCDR2 sequence comprises SEQ ID NO: 17, the HCDR3 sequence comprises SEQ ID NO: 29, the LCDR1 sequence comprises SEQ ID NO: 42, the LCDR2 sequence comprises KAS, and the LCDR3 sequence comprises SEQ ID NO: 61. In some embodiments, the HCDR1 sequence comprises SEQ ID NO: 8, the HCDR2 sequence comprises SEQ ID NO: 19, the HCDR3 sequence comprises SEQ ID NO: 31, the LCDR1 sequence comprises SEQ ID NO: 44, the LCDR2 sequence comprises YTS, and the LCDR3 sequence comprises SEQ ID NO: 63. In some embodiments, the HCDR1 sequence comprises SEQ ID NO: 9, the HCDR2 sequence comprises SEQ ID NO: 20, the HCDR3 sequence comprises SEQ ID NO: 32, the LCDR1 sequence comprises SEQ ID NO: 45, the LCDR2 sequence comprises GAT, and the LCDR3 sequence comprises SEQ ID NO: 64. In some embodiments, the HCDR1 sequence comprises SEQ ID NO: 1, the HCDR2 sequence comprises SEQ ID NO: 21, the HCDR3 sequence comprises SEQ ID NO: 33, the LCDR1 sequence comprises SEQ ID NO: 37, the LCDR2 sequence comprises GTS, and the LCDR3 sequence comprises SEQ ID NO: 65. In some embodiments, the HCDR1 sequence comprises SEQ ID NO: 1, the HCDR2 sequence comprises SEQ ID NO: 21, the HCDR3 sequence comprises SEQ ID NO: 34, the LCDR1 sequence comprises SEQ ID NO: 37, the LCDR2 sequence comprises GTS, and the LCDR3 sequence comprises SEQ ID NO: 65. In some embodiments, the HCDR1 sequence comprises SEQ ID NO: 10, the HCDR2 sequence comprises SEQ ID NO: 22, the HCDR3 sequence comprises SEQ ID NO: 35, the LCDR1 sequence comprises SEQ ID NO: 46, the LCDR2 sequence comprises YAS, and the LCDR3 sequence comprises SEQ ID NO: 66. In some embodiments, the HCDR1 sequence comprises SEQ ID NO: 11, the HCDR2 sequence comprises SEQ ID NO: 23, the HCDR3 sequence comprises SEQ ID NO: 36, the LCDR1 sequence comprises SEQ ID NO: 47, the LCDR2 sequence comprises STS, and the LCDR3 sequence comprises SEQ ID NO: 67. In some embodiments, the VH domain comprises at least 80%, at least 85%, at least 90%, at least 95%, at least 97%, at least 99%, or 100% sequence identity to a sequence selected from SEQ ID NOs: 68-80. In some embodiments, the VL domain comprises at least 80%, at least 85%, at least 90%, at least 95%, at least 97%, at least 99%, or 100% identity to a sequence selected from SEQ ID NOs: 81-93. In some embodiments, the VH domain comprises a portion of a heavy chain comprising at least 80%, at least 85%, at least 90%, at least 95% sequence identity to a sequence selected from SEQ ID NOs: 94-101. In some embodiments, the VL domain comprises a portion of a light chain comprising at least 80%, at least 85%, at least 90%, at least 95% sequence identity to a sequence selected from SEQ ID NOs: 102-105. In some embodiments, the anti-CD122 antibody is a monoclonal antibody. In some embodiments, the anti-CD122 antibody is a humanized antibody. In some embodiments, the anti-CD122 antibody or its antigen-binding fragment thereof comprises IgG-scFv, IgA, IgM, IgE antibody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody. In some embodiments, the anti-CD122 antibody interferes with IL15 binding to the intermediate affinity IL-βγ receptor composed of CD122 beta chain and CD132 gamma chain subunits. In some embodiments, the anti-CD122 antibody diminishes or disrupts IL15-induced signal transduction. In some embodiments, the anti-CD122 antibody interferes with IL2 binding to the intermediate affinity IL-βγ receptor composed of CD122 beta chain and CD132 gamma chain subunits. In some embodiments, the anti-CD122 antibody diminishes or disrupts IL2-induced signal transduction mediated through the intermediate affinity IL-βγ receptor composed of CD122 beta chain and CD132 gamma chain subunits. In some embodiments, the GvHD is acute graft versus host disease (aGvHD). In some embodiments, the administering an effective amount of the anti-CD122 antibody delays an onset of one or more symptoms of aGvHD in the subject. In some embodiments, the administering an effective amount of the anti-CD122 antibody alleviates one or more symptoms of aGvHD in the subject. In some embodiments, the one or more symptoms of aGvHD is selected from a group consisting of: itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss. In some embodiments, the GvHD is chronic graft versus host disease (cGvHD). In some embodiments, the administering an effective amount of the anti-CD122 antibody delays an onset of one or more symptoms of chronic GvHD (cGvHD) in the subject. In some embodiments, the administering an effective amount of the anti-CD122 antibody alleviates one or more symptoms of cGvHD in the subject. In some embodiments, the one or more symptoms of cGvHD is selected from a group consisting of: skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, and changes in hair texture. In some embodiments, the administering an effective amount of the anti-CD122 antibody increases the survival rate of the subject. In some embodiments, the anti-CD122 antibody is administered systemically, locally, intradermally, subcutaneously, or topically. In some embodiments, the anti-CD122 antibody administered systemically is administered by intravenous injection. In some embodiments, the method further comprises administering to the subject an effective amount of a JAK inhibitor, thereby preventing or treating GvHD in the subject. In some embodiments, the anti-CD122 antibody and the JAK inhibitor are co-administered. In some embodiments, the anti-CD122 antibody and the JAK inhibitor are administered separately or sequentially. In some embodiments, the JAK inhibitor is selected from ruxolitinib, abrocitinib, baricitinib, delgocitinib, fedratinib, filgotinib, oclacitinib, pacritinib, peficitinib, tofacitinib, itacitinib and upadacitinib. In some embodiments, the JAK inhibitor is ruxolitinib. In some embodiments, the JAK inhibitor is administered first and the anti-CD122 antibody is administered second. In some embodiments, the administering an effective amount of the anti-CD122 antibody and the JAK inhibitor delays an onset of one or more symptoms of aGvHD or cGvHD in the subject. In some embodiments, the administering an effective amount of the anti-CD122 antibody and the JAK inhibitor alleviates one or more symptoms of aGvHD or cGvHD in the subject. In some embodiments, the administering an effective amount of the anti-CD122 antibody and the JAK inhibitor increases the survival rate of the subject. In some embodiments, the administering an effective amount of the anti-CD122 antibody and the JAK inhibitor increases the survival rate of the subject compared with a subject treated with a JAK inhibitor as a monotherapy. In some embodiments, the anti-CD122 antibody and the JAK inhibitor are administered by the same route of administration. In some embodiments, the anti-CD122 antibody and the JAK inhibitor are administered by separate routes of administration. In some embodiments, the anti-CD122 antibody is administered systemically, locally, intradermally, subcutaneously, or topically, and wherein the JAK inhibitor is administered systemically, locally, intradermally, subcutaneously, or topically. In some embodiments, the anti-CD122 antibody administered systemically is administered by intravenous injection or intraperitoneal injection. In some embodiments, the JAK inhibitor administered systemically is administered by intravenous injection, by enteral administration, or through inhalation. In some embodiments, the JAK inhibitor administered by enteral administration is administered orally.

Described herein, in some aspects, are methods of preventing or treating acute or chronic graft versus host disease (aGvHD or cGvHD) in a subject in need thereof. In an aspect, the methods comprise administering to the subject an effective amount of an anti-CD122 antibody, or its antigen-binding fragment thereof, comprising i) a variable heavy chain (VH) domain and ii) a variable light chain (VL) domain, wherein the VH domain comprises an HCDR1 sequence comprising a sequence selected from SEQ ID NOs: 110-115, an HCDR2 sequence comprising a sequence selected from SEQ ID NOs: 116-123, and an HCDR3 sequence comprising a sequence selected from SEQ ID NOs: 124-130, and the VL domain comprises an LCDR1 sequence comprising a sequence selected from SEQ ID NOs: 131-140, an LCDR2 sequence comprising a sequence selected from SEQ ID NOs: 141-146, and an LCDR3 sequence comprising a sequence selected from SEQ ID NOs: 147-151. In some embodiments, the HCDR1 sequence comprises SEQ ID NO: 110, the HCDR2 sequence comprises SEQ ID NO: 116, the HCDR3 sequence comprises SEQ ID NO: 124, the LCDR1 sequence comprises SEQ ID NO: 131, the LCDR2 sequence comprises SEQ ID NO: 141, and the LCDR3 sequence comprises SEQ ID NO: 147. In some embodiments, the HCDR1 sequence comprises SEQ ID NO: 111, the HCDR2 sequence comprises SEQ ID NO: 116, the HCDR3 sequence comprises SEQ ID NO: 125, the LCDR1 sequence comprises SEQ ID NO: 132, the LCDR2 sequence comprises SEQ ID NO: 141, and the LCDR3 sequence comprises SEQ ID NO: 147. In some embodiments, the HCDR1 sequence comprises SEQ ID NO: 111, the HCDR2 sequence comprises SEQ ID NO: 116, the HCDR3 sequence comprises SEQ ID NO: 125, the LCDR1 sequence comprises SEQ ID NO: 133, the LCDR2 sequence comprises SEQ ID NO: 141, and the LCDR3 sequence comprises SEQ ID NO: 147. In some embodiments, the HCDR1 sequence comprises SEQ ID NO: 111, the HCDR2 sequence comprises SEQ ID NO: 117, the HCDR3 sequence comprises SEQ ID NO: 125, the LCDR1 sequence comprises SEQ ID NO: 132, the LCDR2 sequence comprises SEQ ID NO: 142, and the LCDR3 sequence comprises SEQ ID NO: 148. In some embodiments, the HCDR1 sequence comprises SEQ ID NO: 110, the HCDR2 sequence comprises SEQ ID NO: 118, the HCDR3 sequence comprises SEQ ID NO: 124, the LCDR1 sequence comprises SEQ ID NO: 134, the LCDR2 sequence comprises SEQ ID NO: 143, and the LCDR3 sequence comprises SEQ ID NO: 149. In some embodiments, the HCDR1 sequence comprises SEQ ID NO: 112, the HCDR2 sequence comprises SEQ ID NO: 119, the HCDR3 sequence comprises SEQ ID NO: 126, the LCDR1 sequence comprises SEQ ID NO: 135, the LCDR2 sequence comprises SEQ ID NO: 142, and the LCDR3 sequence comprises SEQ ID NO: 147. In some embodiments, the HCDR1 sequence comprises SEQ ID NO: 113, the HCDR2 sequence comprises SEQ ID NO: 120, the HCDR3 sequence comprises SEQ ID NO: 124, the LCDR1 sequence comprises SEQ ID NO: 136, the LCDR2 sequence comprises SEQ ID NO: 143, and the LCDR3 sequence comprises SEQ ID NO: 148. In some embodiments, the HCDR1 sequence comprises SEQ ID NO: 110, the HCDR2 sequence comprises SEQ ID NO: 119, the HCDR3 sequence comprises SEQ ID NO: 127, the LCDR1 sequence comprises SEQ ID NO: 137, the LCDR2 sequence comprises SEQ ID NO: 143, and the LCDR3 sequence comprises SEQ ID NO: 149. In some embodiments, the HCDR1 sequence comprises SEQ ID NO: 110, the HCDR2 sequence comprises SEQ ID NO: 121, the HCDR3 sequence comprises SEQ ID NO: 128, the LCDR1 sequence comprises SEQ ID NO: 138, the LCDR2 sequence comprises SEQ ID NO: 144, and the LCDR3 sequence comprises SEQ ID NO: 148. In some embodiments, the HCDR1 sequence comprises SEQ ID NO: 114, the HCDR2 sequence comprises SEQ ID NO: 122, the HCDR3 sequence comprises SEQ ID NO: 129, the LCDR1 sequence comprises SEQ ID NO: 139, the LCDR2 sequence comprises SEQ ID NO: 145, and the LCDR3 sequence comprises SEQ ID NO: 150. In some embodiments, the HCDR1 sequence comprises SEQ ID NO: 115, the HCDR2 sequence comprises SEQ ID NO: 123, the HCDR3 sequence comprises SEQ ID NO: 130, the LCDR1 sequence comprises SEQ ID NO: 140, the LCDR2 sequence comprises SEQ ID NO: 146, and the LCDR3 sequence comprises SEQ ID NO: 151. In some embodiments, the VH domain comprises at least 80%, at least 85%, at least 90%, at least 95%, at least 97%, at least 99%, or 100% sequence identity to a sequence selected from SEQ ID NOs: 152-158. In some embodiments, the VL domain comprises at least 80%, at least 85%, at least 90%, at least 95%, at least 97%, at least 99%, or 100% identity to a sequence selected from SEQ ID NOs: 159-165. In some embodiments, the VH domain comprises a portion of a heavy chain comprising at least 80%, at least 85%, at least 90%, at least 95% sequence identity to a sequence selected from SEQ ID NOs: 177-196. In some embodiments, the VL domain comprises a portion of a light chain comprising at least 80%, at least 85%, at least 90%, at least 95% sequence identity to a sequence selected from SEQ ID NOs: 197-199. In some embodiments, the anti-CD122 antibody is a monoclonal antibody. In some embodiments, the anti-CD122 antibody is a humanized antibody. In some embodiments, the anti-CD122 antibody or its antigen-binding fragment thereof comprises IgG-scFv, IgA, IgM, IgE antibody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody. In some embodiments, the anti-CD122 antibody interferes with IL15 binding to the intermediate affinity IL-βγ receptor composed of CD122 beta chain and CD132 gamma chain subunits. In some embodiments, the anti-CD122 antibody diminishes or disrupts IL15-induced signal transduction. In some embodiments, the anti-CD122 antibody interferes with IL2 binding to the intermediate affinity IL-βγ receptor composed of CD122 beta chain and CD132 gamma chain subunits. In some embodiments, the anti-CD122 antibody diminishes or disrupts IL2-induced signal transduction mediated through the intermediate affinity IL-βγ receptor composed of CD122 beta chain and CD132 gamma chain subunits. In some embodiments, the GvHD is acute graft versus host disease (aGvHD). In some embodiments, the administering an effective amount of the anti-CD122 antibody delays an onset of one or more symptoms of aGvHD in the subject. In some embodiments, the administering an effective amount of the anti-CD122 antibody alleviates one or more symptoms of aGvHD in the subject. In some embodiments, the one or more symptoms of aGvHD comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss. In some embodiments, the GvHD is chronic graft versus host disease (cGvHD). In some embodiments, the administering an effective amount of the anti-CD122 antibody delays an onset of one or more symptoms of chronic GvHD (cGvHD) in the subject. In some embodiments, the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, or changes in hair texture. In some embodiments, the administering an effective amount of the anti-CD122 antibody increases the survival rate of the subject. In some embodiments, the anti-CD122 antibody is administered systemically, locally, intradermally, subcutaneously, or topically. In some embodiments, the anti-CD122 antibody administered systemically is administered by intravenous injection. In some embodiments, the method further comprises administering to the subject an effective amount of a JAK inhibitor, thereby preventing or treating GvHD in the subject. In some embodiments, the anti-CD122 antibody and the JAK inhibitor are co-administered. In some embodiments, the anti-CD122 antibody and the JAK inhibitor are administered separately or sequentially. In some embodiments, the JAK inhibitor is selected from ruxolitinib, abrocitinib, baricitinib, delgocitinib, fedratinib, filgotinib, oclacitinib, pacritinib, peficitinib, tofacitinib, itacitinib and upadacitinib. In some embodiments, the JAK inhibitor is ruxolitinib. In some embodiments, the JAK inhibitor is administered first and the anti-CD122 antibody is administered second. In some embodiments, the administering an effective amount of the anti-CD122 antibody and the JAK inhibitor delays an onset of one or more symptoms of aGvHD or cGvHD in the subject. In some embodiments, the administering an effective amount of the anti-CD122 antibody and the JAK inhibitor alleviates one or more symptoms of aGvHD or cGvHD in the subject. In some embodiments, the administering an effective amount of the anti-CD122 antibody and the JAK inhibitor increases the survival rate of the subject. In some embodiments, the administering an effective amount of the anti-CD122 antibody and the JAK inhibitor increases the survival rate of the subject compared with a subject treated with a JAK inhibitor as a monotherapy. In some embodiments, the anti-CD122 antibody and the JAK inhibitor are administered by the same route of administration. In some embodiments, the anti-CD122 antibody and the JAK inhibitor are administered by separate routes of administration. In some embodiments, the anti-CD122 antibody is administered systemically, locally, intradermally, subcutaneously, or topically, and wherein the JAK inhibitor is administered systemically, locally, intradermally, subcutaneously, or topically. In some embodiments, the anti-CD122 antibody administered systemically is administered by intravenous injection or intraperitoneal injection. In some embodiments, the JAK inhibitor administered systemically is administered by intravenous injection, by enteral administration, or through inhalation. In some embodiments, the JAK inhibitor administered by enteral administration is administered orally.

Described herein, in some aspects, are methods of preventing or treating acute or chronic graft versus host disease (aGvHD or cGvHD) in a subject in need thereof. In an aspect, a method of treating graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 152, and an LCDR1, LCDR2, LCDR3 of a light chain variable domain comprising the amino acid sequence set forth in SEQ ID NO: 159, wherein the administering treats one or more symptoms of acute GvHD or chronic GvHD. In an aspect, a method of preventing graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 152, and an LCDR1, LCDR2, LCDR3 of a light chain variable domain comprising the amino acid sequence set forth in SEQ ID NO: 159, wherein the administering prevents one or more symptoms of acute GvHD or chronic GvHD. In an aspect, a method of treating graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 160, wherein the administering treats one or more symptoms of acute GvHD or chronic GvHD. In an aspect, a method of preventing graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 160, wherein the administering prevents one or more symptoms of acute GvHD or chronic GvHD. In an aspect, a method of treating graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 161, wherein the administering treats one or more symptoms of acute GvHD or chronic GvHD. In an aspect, a method of preventing graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 161, wherein the administering prevents one or more symptoms of acute GvHD or chronic GvHD. In an aspect, a method of treating graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 156, and an LCDR1, LCDR2, LCDR3 of a light chain variable domain comprising the amino acid sequence set forth in SEQ ID NO: 163, wherein the administering treats one or more symptoms of acute GvHD or chronic GvHD. In an aspect, a method of preventing graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 156, and an LCDR1, LCDR2, LCDR3 of a light chain variable domain comprising the amino acid sequence set forth in SEQ ID NO: 163, wherein the administering prevents one or more symptoms of acute GvHD or chronic GvHD. In an aspect, a method of treating graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 158, and an LCDR1, LCDR2, LCDR3 of a light chain variable domain comprising the amino acid sequence set forth in SEQ ID NO: 165, wherein the administering treats one or more symptoms of acute GvHD or chronic GvHD. In an aspect, a method of preventing graft versus host disease (GvHD) in a subject in need thereof comprises administering to the subject an effective amount of an anti-CD122 antibody, or its antigen-binding fragment thereof, comprising a HCDR1, HCDR2, HCDR3 of a heavy chain variable domain comprising the amino acid sequence set forth in SEQ ID NO: 158, and an LCDR1, LCDR2, LCDR3 of a light chain variable domain comprising the amino acid sequence set forth in SEQ ID NO: 165, wherein the administering prevents one or more symptoms of acute GvHD or chronic GvHD.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an acute GvHD study design for a dose-ranging investigation of an anti-CD122 antibody compared with ruxolitinib treatment.

FIG. 2 shows a graph of survival duration in an acute GvHD mouse model comparing effectiveness of anti-CD122 antibody monotherapy treatment results using different dosages of an anti-CD122 antibody (Antibody 1) compared with ruxolitinib monotherapy effectiveness.

FIG. 3 shows an acute GvHD study design for single therapy and combination therapy treatment test groups.

FIG. 4 shows a graph of survival duration in an acute GvHD mouse model comparing effectiveness of anti-CD122 antibody (Antibody 1) plus ruxolitinib treatment versus ruxolitinib monotherapy versus vehicle-treated control.

FIG. 5 shows an acute GvHD study design for single therapy and combination therapy treatment test groups.

FIG. 6 shows a graph of survival duration in an acute GvHD mouse model comparing effectiveness of: i) anti-CD122 antibody (Antibody 2) plus ruxolitinib treatment, ii) anti-CD122 antibody (Antibody 3) plus ruxolitinib treatment, and iii) ruxolitinib monotherapy, versus iv) vehicle-treated control.

FIG. 7 shows an allogeneic chronic GvHD study design for assessing anti-CD122 antibody and ruxolitinib treatment effectiveness alone and in combination.

### DETAILED DESCRIPTION

Immune cell responses are often context dependent and in some cases are influenced by signals from their environment through a variety of receptor-ligand interactions. For instance, in various embodiments, these signals amplify and modify a T cell receptor (TCR) signal received by antigenic stimulation in a resting naïve or memory T cell, regulate T cell proliferation and differentiation in recently activated T cells, or control effector functions in particular somatic environments. IL2 and IL15 share similar and contrasting roles in regulation of T cell function. As a non-limiting example, both IL2 and IL15 are involved in T cell differentiation. IL2 promotes the differentiation of immature T cells into regulatory T cells, which thereby are capable of suppressing other T cells that could attack normal healthy cells in the body. IL2 signaling is involved in peripheral tolerance through the elimination of self-reactive T cells by way of the activation-induced cell death (AICD) pathway. In some cases, IL2 also promotes the differentiation of immature T cells into either effector T cells or into memory T cells when an initial T cell is stimulated by an antigen. IL2 has also been demonstrated to enhance the activity of both cytotoxic T cells and natural killer (NK) cells. In some cases, IL15 regulates the activation and proliferation of T cells and NK cells. In contrast to IL2 signaling, in some cases, IL15 signaling inhibits IL2-mediated AICD by eliciting antiapoptotic actions. In some instances, IL15 stimulates the persistence of memory phenotype CD8+ T cells that are involved in the elimination of invading pathogens, thereby protecting the subject against infection.

IL2 and IL15 have distinct means for initiating signaling through their receptors. IL2 is a predominantly secreted cytokine that either in its soluble form or linked to extracellular matrix can bind to heterodimeric (IL2Rβ/IL2Rγ) and heterotrimeric (IL2Rα/IL2Rβ/IL2Rγ) receptor complexes both involving CD122 on the surface of activated cells. IL15, mainly secreted along with IL15Rα, is primarily membrane bound and induces signaling in the context of cell-cell contacts, at the immunological synapse. IL15Ru presents membrane bound IL15 in the trans configuration to neighboring CD8+ T cells and NK cells. Despite these differences in the mechanisms of the initiation of ligand-mediated signaling, once activated, IL2 receptor complexes and IL15 receptor complexes activate shared molecular pathways including the JAK1/JAK3/STAT5, the PI3K, and the MAPK signal transduction pathways. IL2RP binds to JAK1 and IL2Ry binds to JAK3. Ligand binding of IL2R and IL15R can result in activation of JAK kinases and phosphorylation of tyrosine residues on IL2RP and IL2Ry. Tyrosine phosphorylation of IL2RP permits recruitment of STAT5A, STAT5B, STAT3, and/or SHC1. Following their recruitment to IL2R or IL15R, STAT5 and/or SHC1 proteins can be phosphorylated by one or more JAK proteins. Tyrosine phosphorylation of STAT5 permits protein dimerization, subsequent nuclear translocation and STAT5-mediated gene transcription. Tyrosine phosphorylation of SHC1 permits recruitment of GRB2 and SOS to facilitate activation of the Raf-ERK MAP kinase signaling cascade. In some cases, activation of these pathways modulates gene transcription to regulate apoptosis, proliferation, or differentiation of immune cells. Functioning in both IL2 receptors and IL15 receptors, CD122 serves critical roles in these various capacities.

Disclosed herein, in certain aspects, are anti-CD122 antibodies, and pharmaceutical compositions which comprise the anti-CD122 antibodies. In some embodiments, also disclosed herein are methods used for targeting CD122-expressing tissues and cells with an anti-CD122 antibody described herein.

### Graft Versus Host Disease (GvHD)

Graft versus host disease (GvHD) is a condition which often occurs following an allogeneic transplant. In GvHD, donated multipotent hematopoietic stem cells (typically derived from bone marrow, peripheral blood stem cells, umbilical cord stem cells, or stem cells of other sources) recognize the recipient's cells and organs as foreign, and these donated cells and their derivatives attack the host's body. There are two recognized forms of GvHD: acute graft versus host disease (aGvHD) and chronic graft versus host disease (cGvHD). A host subject receiving an allogeneic transplant is at risk for developing aGvHD, cGvHD, or both conditions. Acute GvHD and chronic GvHD can affect the skin, the gastrointestinal (GI) tract, the liver, and other tissues and organs. Pathogenic inflammation can occur in various affected organs in subjects presenting with aGvHD and/or cGvHD.

Acute GvHD occurs in up to 50% of transplant recipients with onset typically occurring within 3 months of transplant. Moderate-to-severe aGvHD develops in about 20-50% of recipients of an HLA-identical sibling allogeneic stem cell transplant. Estimates of mortality directly attributable to aGvHD or treatment thereof occurs in approximately 10-20% of patients. In aGvHD, a combination of symptoms in various organs is often involved including skin (rash), GI tract (vomiting and/or diarrhea), and liver (e.g., jaundice). The skin is the most commonly affected site in aGvHD and symptoms often manifest as a rash resembling a sunburn with blistering or peeling and often affect the back, shoulders, ears, neck, palms of hands, and soles of feet of the host. In some patients, the most common manifestation of aGvHD is a maculopapular rash, typically occurring at or near the time of white blood cell engraftment. This rash typically involves the nape of the neck, ears, shoulders, palms of the hands, and soles of the feet initially. The rash can later spread to involve the entire integument. Histologic examination of the skin often reveals changes in both epidermal and dermal layers. Characteristic findings of aGvHD pathology in the skin include exocytosed lymphocytes, dyskeratotic epidermal keratinocytes, follicular involvement, satellite lymphocytes near dyskerototic epidermal keratinocytes, and dermal perivascular lymphocytic infiltration. A consistent aGvHD pathological feature in the skin is apoptosis at the base of crypts.

aGvHD in the GI tract often causes abdominal pain, diarrhea, persistent nausea and/or vomiting, and a loss of appetite or a feeling of satiety after eating a small amount of food. A diagnosis of GI involvement in aGvHD may require pathological evaluation of biopsied tissue. An extent of GI involvement in aGvHD can be measured according to volume of diarrhea in the subject per day.

In some cases, aGvHD also affects the liver, causing symptoms such as dark urine, jaundice, and elevated liver enzymes in the blood. An extent of liver involvement in aGvHD can be measured according to serum total bilirubin levels in a patient and also if bilirubin levels in the patient rise over time.

The stage of liver involvement in aGvHD can be combined with assessments of the stage of cutaneous and GI tract involvement to determine an overall severity grade for aGvHD. Less commonly involved in aGvHD are the hematopoietic system, eyes, lungs, and/or kidneys. Pathological alterations in these organ systems are not used to establish an initial diagnosis of aGvHD, but may be informative of overall severity grade for aGvHD once aGvHD diagnosis has already been established. Hematopoietic involvement in aGvHD can manifest as thymic atrophy, a cytopenia (e.g., thrombocytopenia), and/or hypogammaglobulinemia (e.g., IgA deficiency). Involvement in the eyes in aGvHD can lead to photophobia, hemorrhagic conjunctivitis, and an inability to completely close the eyes. Kidney involvement in aGvHD can present as nephritis or nephrotic syndrome. Lung involvement in aGvHD can manifest itself as interstitial pneumonitis. A diagnosis of aGvHD can be made on clinical grounds alone in a subject that presents with a typical aGvHD rash, abdominal cramps with diarrhea, and serum bilirubin concentrations that rise within the first 100 days following transplantation.

Occurrence of prior aGvHD is a main risk factor for development of cGvHD. The pathogenesis of cGvHD is complex and includes tissue damage, unusual antigen presentation and aberrant myeloid and lymphoid interactions. The initial phase of cGvHD includes an effect of early post-transplant inflammation and tissue injury. Excessive release of inflammatory cytokines activates antigen-presenting cells which stimulate the activation of donor alloreactive T cells having enhanced T cell effector lineages. Macrophages are also sequestered in affected tissues. Following this initial phase, in some cases, cGvHD progresses to the presence of chronic inflammation and dysregulation of the immune system operating outside of the normal regulatory immune responses. Further progression of cGvHD is evident as aberrant repair mechanisms lead to a release of profibrotic mediators via monocytes and macrophages. In some cases, this causes fibroblast activation, collagen deposition, and ultimately fibrosis.

Symptoms of cGvHD in the skin can include rash, raised, or discolored skin areas, and skin thickening or tightening. Signs of cGvHD in the liver include abdominal swelling, a yellow discoloration of the eyes and or skin (jaundice), and abnormal blood test results including elevated liver enzymes. Signs of cGvHD in the eyes include dry eyes or changes in vision. Signs of cGvHD in the mouth and oral region include dry mouth, white patches on the inside of the mouth, and pain or sensitivity to spicy foods. Signs of pulmonary cGvHD include shortness of breath, dry cough, or alterations seen on a chest X-ray. Signs of cGvHD in the GI tract include difficulty swallowing, pain with swallowing, or weight loss. Signs of neuromuscular cGvHD include fatigue, or muscle weakness or pain. In some cases, cGvHD affects the vagina or vulva resulting in vaginal dryness or pain. cGvHD affecting the connective tissue often results in tightness in the joints and a decreased range of bodily motion. Chronic GvHD develops in up to 40% of transplant recipients and onset typically occurs after about 100 days following transplant. In addition to involvement of the skin, GI tract, and liver, cGvHD symptoms can involve dysfunction in the lungs, mucosal surfaces (e.g., eyes, mouth, and/or GI tract), muscles, and joints (e.g., connective tissues).

In some cases, GvHD is diagnosed during a physical examination by a medical practitioner by observation of GvHD-related symptoms and/or by evaluating the results of biopsies and clinical lab tests. In the case of cGvHD, symptoms sometimes present as vague or even transitory which may make a diagnosis of cGvHD possible only following the exclusion of other potential causes of symptomatology. Although the manifestation of cGvHD symptoms can be heterogeneous at onset, certain features are termed diagnostic features sufficient to establish a diagnosis of cGvHD. Diagnostic features of cGvHD include sclerosis, lichen-planus-like lesions, poikiloderma, esophageal webs, and fasciitis and bronchiolitis obliterans. In contrast, distinctive features which are highly suggestive of cGvHD but are not sufficient by themselves to establish diagnosis include oral ulcers and atrophy, onchodystrophy, and sicca syndrome. Distinctive features of cGvHD such as those listed above may be confirmed as cGvHD through biopsy or by other diagnostic test criteria. The most frequent sites of pathology involved at the initial diagnosis of cGvHD are skin, mouth (e.g., lichen-planus-like lacy buccal involvement, xerostomia from salivary gland dysfunction, food sensitivity, oral pain, erythema, and/or non-healing mouth ulcers), liver, and eye. Less frequently involved sites of pathology at the initial diagnosis of cGvHD are GI tract (e.g., as evidenced by unexplained weight loss), lung, esophagus, female genital tract, and joints.

To attempt to mitigate the risk of GvHD occurrence, the best HLA-matched donor is selected for the transplant into the host. Additionally, prophylactic (preventative) treatments often aimed at suppressing the immune system are regularly initiated following transplant. These treatments are aimed at decreasing the ability of the donor's cells and derivatives thereof of initiating an active immune response against host cells, tissues, and organs. Fungal, bacterial, and viral infections are major risks for subjects undergoing an immunosuppressive prophylactic treatment regimen as the host's body will maintain a decreased ability to fight infection while under immunosuppression. Prophylactic antibiotics, antifungals and antiviral medicines are often administered during immunosuppressive therapy to decrease risks of infection.

Treatments currently used in an attempt to ameliorate symptoms in aGvHD include administration of corticosteroids, ruxolitinib, sirolimus, mycophenolate mofetil (CellCept), mycophenolate sodium (Myfortic), or antithymocyte globulin. These drugs may be administered orally and/or intravenously. A different therapeutic approach to treat aGvHD is extracorporeal photopheresis, involving removal and separation of leukocytes from the affected subject and then exposure of those cells to ultraviolet irradiation in the presence of a photosensitizing agent prior to reinfusion of the treated cells into the subject. TNFα inhibitors (e.g., adalimumab or infliximab) have been attempted as therapeutics to treat or ameliorate symptoms of aGvHD. In a first-line standard of care therapy including treatment using one or more corticosteroids, approximately 30-50% of patients have an inadequate response to treatment. In patients with grade 3 and grade 4 aGvHD disease, following solely a first-line standard of care therapy leads to a 2-year mortality rate of >70%.

In contrast, treatments currently used to attempt to reduce symptoms of cGvHD include various forms of immunosuppressive therapies. In addition, the heterogenous nature of cGvHD has led to a variety of treatments that can be tried, such as extracorporeal photopheresis, although very few of which are approved by the FDA to treat the condition. Approved treatments include, ruxolitinib, belumosudil, and ibrutinib. A first-line standard of care therapy for cGvHD includes treatment with one or more corticosteroids, however there is limited success following this first-line therapy. Approximately 50-60% of patients undergoing corticosteroid treatment for cGvHD will require initiation of a second-line therapy within 2 years.

In various aspects of methods provided herein, aGvHD typically occurs in the early post-transplantation period. The initial signs and symptoms often occur during the time of white blood cell engraftment. Although initial definitions of aGvHD required onset of symptoms before 100 days post transplantation, the current consensus uses clinical findings rather than a set time period to differentiate aGvHD from cGvHD. The skin, gastrointestinal tract, and liver are the principal target organs for aGvHD. Other affected organs include the hematopoietic system, the eyes, kidneys, and lungs. Diagnosis of aGvHD is often made in post-hematopoietic cell transplantation in a patient having a rash, abdominal cramps with diarrhea, and rising serum bilirubin concentration during the first 100 days following transplantation. However, histologic confirmation via skin and/or gastrointestinal biopsy is often undertaken. Biomarkers for aGvHD include suppression of tumorigenicity 2 (ST2), regenerating islet-derived 3-alpha (REG3alpha), and tumor necrosis factor receptor 1 (TNFR1).

In contrast, cGvHD presents with a variety of clinical features that often resemble autoimmune and other immunologic disorders, such as scleroderma, Sjogren's syndrome, primary biliary cirrhosis, and bronchiolitis obliterans. In some cases, clinical manifestation is widespread. Alternatively, symptoms are restricted to a single organ or site. The most frequent symptoms include skin involvement (resembling lichen planus or cutaneous scleroderma), dry oral mucosa, gastrointestinal tract ulcerations and sclerosis, elevated serum bilirubin, and bronchiolitis obliterans. Promising serum biomarkers for cGvHD include CXCL9, ST2, matrix metalloproteinase-3, osteopontin, CXCL10, CXCL11, and CD163.

aGvHD Treatment with an Anti-CD122 Antibody

Disclosed herein, in certain aspects, are anti-CD122 antibodies for use in methods of preventing and/or treating acute graft versus host disease. Methods for prophylaxis of aGvHD or treatment for aGvHD comprising administration of an anti-CD122 antibody are described herein. In some embodiments, the methods of prophylaxis or treatment comprise contacting a plurality of cells in the subject with an anti-CD122 antibody. In some embodiments, the anti-CD122 antibody functions as an IL15R inhibitor in the method of treatment. In some embodiments, the anti-CD122 antibody functions as an intermediate affinity IL-βγ receptor inhibitor in the method of treatment. The intermediate affinity IL-βγ receptor is composed of CD122 beta chain and CD132 gamma chain subunits. In some embodiments, the anti-CD122 antibody functions as an IL2R inhibitor in the method of treatment. In some embodiments, the anti-CD122 antibody functions as an IL15R inhibitor and an IL2R inhibitor in the method of treatment. In some embodiments, the anti-CD122 antibody inhibits the intermediate affinity IL-βγ receptor composed of CD122 beta chain and CD132 gamma chain subunits. In some embodiments, IL2-mediated signaling through the intermediate affinity IL-βγ receptor is inhibited in the method of treatment. In some embodiments, IL15-mediated signaling is inhibited in the method of treatment. In some embodiments, IL15-mediated signaling through the intermediate affinity IL-βγ receptor is inhibited in the method of treatment. In some embodiments, the anti-CD122 antibody functions as an IL15R inhibitor in the method of use. In some embodiments, the anti-CD122 antibody functions as an intermediate affinity IL-βγ receptor inhibitor in the method of use.

In some embodiments, the anti-CD122 antibody functions as an IL2R inhibitor in the method of use. In some embodiments, the anti-CD122 antibody functions as an intermediate affinity IL-βγ receptor inhibitor in the method of use. In some embodiments, the anti-CD122 antibody functions as an IL15R inhibitor and an IL2R inhibitor in the method of use. In some embodiments, the IL15R inhibitor is an IL15Rβinhibitor. The IL15Rβ subunit is also known as CD122. In some embodiments, the IL2R inhibitor is an IL2Rβ inhibitor. The IL2Rβ subunit is also known as CD122. In some embodiments, the anti-CD122 antibody functions as an inhibitor of the intermediate affinity IL-βγ receptor composed of CD122 beta chain and CD132 gamma chain subunits. In some embodiments, the anti-CD122 antibody functions as an inhibitor IL2 signaling and/or IL15 signaling in cells expressing the intermediate affinity IL-βγ receptor. In some embodiments, the anti-CD122 antibody interferes with IL15 binding to the IL15R and/or interferes with IL2 binding to the IL2R. In some instances, interfering with IL15 binding to the IL15R comprises competitive binding of the antibody to the IL15R compared to IL15 binding to the IL15R. In some instances, interfering with IL15 binding to the IL15R comprises competitive inhibition of the anti-CD122 antibody to endogenous IL15-mediated signaling. In some instances, interfering with IL2 binding to the IL2R comprises competitive binding of the anti-CD122 antibody to the IL2R compared to IL2 binding to the IL2R. In some instances, interfering with IL2 binding to the intermediate affinity IL-βγ receptor comprises competitive binding of the anti-CD122 antibody to the intermediate affinity IL-βγ receptor compared to IL2 binding to the intermediate affinity IL-βγ receptor. In some instances, interfering with IL2 binding to the IL2R comprises competitive inhibition of the anti-CD122 antibody to endogenous IL2-mediated signaling. In some instances, the anti-CD122 antibody interferes with IL2 and/or IL15 binding to the intermediate affinity IL-βγ receptor by binding to CD122 with a stronger affinity than IL2 and/or IL15 binds to intermediate affinity IL-βγ receptor. In some embodiments, the anti-CD122 antibody functions as an inhibitor of IL2-mediated signal transduction and/or IL15-mediated signal transduction in i) lymphocytes or progenitors thereof and/or ii) NK cells or progenitors thereof. In some embodiments, IL2 signaling in cells of the subject expands CD4+ helper T cells and regulatory T cells (Tregs). In some embodiments, inhibiting IL2 signaling in cells of the subject selectively affects IL2-mediated expansion of CD4+ helper T cells. In some embodiments, IL15 signaling in cells of the subject supports the development of memory T cells and NK cells. In some embodiments, inhibiting IL15 signaling in cells of the subject selectively affects IL2-mediated support of memory T cell development. In some instances in subjects having received an alloHSCT, IL15 signaling in cells of the subject is more efficient than IL2 signaling in cooperating with IL21 to boost the expansion and effector function of donor-derived CD8+ T cells. In some embodiments of methods described herein, inhibiting IL15 signaling in cells of the subject reduces expansion and effector function of donor-derived CD8+ T cells. In some embodiments, inhibiting IL15 signaling and/or IL2 signaling in cells of the subject reduces the activation and expansion of alloreactive T cells from secondary lymphoid tissues (SLTs). In some embodiments, inhibiting IL15 signaling and/or IL2 signaling in cells of the subject reduces recruitment of alloreactive T cells into aGvHD target tissues from the blood. In some embodiments, inhibiting IL15 signaling and/or IL2 signaling in cells of the subject reduces the activation and expansion of alloreactive T cells locally maintained within diseased tissue. In some instances, subjects with aGvHD avoid T cell exhaustion by continued activation and proliferation of alloreactive T cells following establishing of local sources of T cell progenitors within diseased tissue. In some embodiments of methods described herein, inhibiting IL15 signaling and/or IL2 signaling in cells of the subject reduces the activation and expansion of alloreactive T cells locally maintained within diseased tissue and results in an increased percentage of exhausted T cells compared to no inhibition of IL15 signaling and/or IL2 signaling. In some instances, a tissue-resident Tcf7-expressing (protein product is TCF-1) T cell subset population is involved in intratissue aGvHD maintenance. In some embodiments of methods described herein, inhibiting IL15 signaling and/or IL2 signaling in cells of the subject reduces activation and proliferation of tissue-resident Tcf7-expressing T cell within affected tissues of the subject. In some embodiments of methods described herein, inhibiting IL15 signaling and/or IL2 signaling in cells of the subject reduces proliferation of NK cells.

In some embodiments, the administering of the anti-CD122 antibody inhibits IL2 and/or IL15 signaling. In some embodiments, the administering of the anti-CD122 antibody inhibits IL2 signaling. In some embodiments, the administering of the anti-CD122 antibody inhibits IL15 signaling. In some embodiments, the administering of the anti-CD122 antibody inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the anti-CD122 antibody disrupts or diminishes an IL15/IL15Ra complex from binding to an IL15Rβ/IL15Rγ complex. In some embodiments, the anti-CD122 antibody disrupts or diminishes IL2 from binding to an IL2Rβ/IL2Rγ complex. In some embodiments, the anti-CD122 antibody disrupts or diminishes both IL2 and IL15 from binding to an intermediate affinity IL-βγ receptor. In some embodiments, the effective amount of the anti-CD122 antibody does not significantly disrupt IL2 from binding to a high affinity IL2Rα/IL2Rβ/IL2Rγ complex. In some embodiments, the effective amount of the anti-CD122 antibody does not significantly disrupt IL2 from signaling through a high affinity IL2Rα/IL2Rβ/IL2Rγ complex.

In some embodiments, the anti-CD122 antibody targets CD122 in CD122-expressing cells in a subject following administering of the antibody. In some embodiments, targeting CD122 with an anti-CD122 antibody reduces the ability of IL2 to bind to an IL2R composed of a CD122 subunit and reduces the ability to elicit an amount of IL2-mediated signal transduction. In some embodiments, targeting CD122 with an anti-CD122 antibody reduces the ability of IL15, or IL15 bound to IL15Rα, to bind to an IL15R composed of a CD122 subunit and reduces the ability to elicit an amount of IL15-mediated signal transduction. In some embodiments, targeting CD122 with an anti-CD122 antibody reduces the ability of IL2 to bind to intermediate affinity IL2R composed of beta and gamma receptor subunits. In some embodiments, targeting CD122 with an anti-CD122 antibody reduces the ability of IL15, or IL15 bound to IL15Rα, to bind to intermediate affinity IL15R composed of beta and gamma receptor subunits. In some embodiments, targeting CD122 with an anti-CD122 antibody reduces the ability of IL2 and/or IL15 from binding to intermediate affinity IL-βγ receptor and reduces the ability to elicit an extent of IL2-mediated and/or IL15-mediated signal transduction. In some embodiments, targeting CD122 with an anti-CD122 antibody reduces the ability of IL2 and/or IL15 from binding to high affinity IL-βγ receptor (composed of an alpha/beta/gamma subunit heterotrimer) and reduces the ability to elicit an extent of IL2-mediated and/or IL15-mediated signal transduction. In some embodiments, targeting CD122 with an anti-CD122 antibody reduces the ability of IL2 and/or IL15 from binding to intermediate affinity IL-βγ receptor and reduces the ability to elicit an extent of IL2-mediated and/or IL15-mediated signal transduction more effectively than acting through high affinity IL-αβγ receptor. In some embodiments, this preferential effect of targeting intermediate affinity IL-βγ receptor with an anti-CD122 antibody can be due to the specific affinity of the anti-CD122 antibody to CD122 and intermediate affinity IL-βγ receptor composed of CD122. Intermediate-affinity heterodimeric IL-βγ receptor have a K_{d}˜10⁻⁹ M for IL2 or IL15, whereas the high-affinity heterotrimer IL-αβγ receptor combinations have a K_{d}˜10⁻¹¹ M for IL2 or IL15. In some embodiments, selecting an anti-CD122 antibody with a particular binding affinity for CD122 close to the natural affinity for IL2 or IL15 for IL-βγ receptor, or in between the natural affinity for IL2 or IL15 for IL-βγ receptor compared to the natural affinity for IL12 or IL15 for their respective IL-αβγ receptors is a means of selectively targeting particular cells or cell types. In some embodiments, cells or cell types with strong expression of intermediate affinity IL2R and/or IL15R, but lacking strong expression of high affinity IL-αβγ receptor can be selectively targeted by an anti-CD122 antibody with a K_{d}˜10⁻⁹ to 10⁻¹⁰ M. In some embodiments, cells or cell types expressing high affinity IL-αβγ receptor can be less affected by an anti-CD122 antibody with a K_{d}˜10⁻⁹ to 10⁻¹⁰ M, as the antibody may not substantially interference with ligand binding to the high affinity receptor. In some embodiments, the anti-CD122 antibody administered to the subject is selected based on its binding affinity to CD122. In some embodiments, targeting CD122 with an anti-CD122 antibody reduces the ability of IL15, or IL15 bound to IL15Rα, to bind to an IL15R composed of a CD122 subunit and reduces the ability to elicit an amount of IL15-mediated signal transduction. In some embodiments, the anti-CD122 antibody binds to an epitope within the extracellular domain of CD122. In some embodiments, the anti-CD122 antibody binds to a linear epitope in CD122. In some embodiments, the anti-CD122 antibody binds to a conformational epitope in CD122. In some embodiments, the epitope overlaps with a binding region of IL2 and/or IL15 on the extracellular domain of CD122.

In some embodiments, the anti-CD122 antibody directly inhibits initiation and/or maintenance of IL2-mediated signal transduction and/or IL15-mediated signal transduction. In some embodiments, targeting CD122 with an anti-CD122 antibody can reduce the ability of IL2 to bind to an IL2R composed of a CD122 subunit and can reduce the ability to elicit an amount of IL2-mediated signal transduction. In some embodiments, targeting CD122 with an anti-CD122 antibody can reduce the ability of IL15 to bind to an IL15R composed of a CD122 subunit and can reduce the ability to elicit an amount of IL15-mediated signal transduction. In some embodiments, targeting CD122 with an anti-CD122 antibody inhibits IL2-mediated signal transduction, IL15-mediated signal transduction, or IL2-mediated signal transduction and IL15-mediated signal transduction. In some embodiments, the CD122 epitope in which the anti-CD122 antibody binds overlaps with a binding region of IL2 and/or IL15 on the extracellular domain of CD122. In some embodiments, the CD122 epitope in which the anti-CD122 antibody binds does not overlap with a binding region of IL2 and/or IL15 on the extracellular domain of CD122.

In some embodiments, the anti-CD122 antibody indirectly inhibits initiation and/or maintenance of IL2-mediated signal transduction and/or IL15-mediated signal transduction. In some embodiments, targeting CD122 with an anti-CD122 antibody does not significantly reduce the ability of IL2 to bind to an IL2R composed of a CD122 subunit, but can reduce the ability to elicit an amount of IL2-mediated signal transduction. In some embodiments, targeting CD122 with an anti-CD122 antibody does not significantly reduce the ability of IL15 to bind to an IL15R composed of a CD122 subunit, but can reduce the ability to elicit an amount of IL15-mediated signal transduction. In some embodiments, targeting CD122 with an anti-CD122 antibody inhibits IL2-mediated signal transduction, IL15-mediated signal transduction, or IL2-mediated signal transduction and IL15-mediated signal transduction. In some embodiments, targeting CD122 with an anti-CD122 antibody does not disrupt IL2 or IL15 from binding to IL2R or IL15R respectively, but prevents the IL2R complex or the IL15R complex from effectively initiating and/or maintaining IL2-mediated signal transduction or IL15-mediated signal transduction respectively. In some embodiments, the CD122 epitope in which the anti-CD122 antibody binds does not overlap with a binding region of IL2 and/or IL15 on the extracellular domain of CD122.

In some embodiments, methods described herein involve contacting one or more cells in the subject with an anti-CD122 antibody to prevent or treat aGvHD. In some embodiments, methods using the anti-CD122 antibody inhibit IL2 signaling in cells contacted with the anti-CD122 antibody. In some embodiments, methods using the anti-CD122 antibody inhibit IL15 signaling in cells contacted with the anti-CD122 antibody. In some embodiments, methods using the anti-CD122 antibody inhibit IL2 and IL15 signaling in cells contacted with the anti-CD122 antibody. In some embodiments, a plurality of cells in the subject expressing CD122 exhibit IL2 signaling inhibition after being contacted with the anti-CD122 antibody. In some embodiments, a plurality of cells in the subject expressing CD122 exhibit IL15 signaling inhibition after being contacted with the anti-CD122 antibody. In some embodiments, a plurality of cells in the subject expressing CD122 exhibit IL2 and IL15 signaling inhibition after being contacted with the anti-CD122 antibody. In some embodiments, a plurality of cells in the subject expressing CD122 are targeted by a mechanism of cell-mediated immune defense after being contacted with the anti-CD122 antibody. In some embodiments, the mechanism of cell-mediated immune defense is antibody-dependent cellular cytotoxicity (ADCC). In some embodiments, contacting a plurality of cells in the subject with the anti-CD122 antibody inhibits IL2-mediated cellular proliferation. In some embodiments, contacting a plurality of cells in the subject with the anti-CD122 antibody inhibits IL15-mediated cellular proliferation. In some embodiments, contacting a plurality of cells in the subject with the anti-CD122 antibody inhibits IL2-mediated cellular proliferation and IL15-mediated cellular proliferation. In some embodiments, the IL2-mediated cellular proliferation and/or IL15-mediated cellular proliferation affects proliferation of lymphocytes in the subject. In some embodiments, memory T cell proliferation is reduced in the subject following administration of the anti-CD122 antibody. In some embodiments, CD4+ T cell proliferation is reduced in the subject following administration of the anti-CD122 antibody. In some embodiments, CD8+ T cell proliferation is reduced in the subject following administration of the anti-CD122 antibody.

In another aspect, a method of prophylaxis for aGvHD comprises administering an effective amount of an anti-CD122 antibody described herein.

In some embodiments, methods of prophylaxis and/or treatment of aGvHD comprising administration of an anti-CD122 antibody alter a level of a serum biomarker for aGvHD. In some embodiments, methods of prophylaxis and/or treatment of aGvHD comprising administration of an anti-CD122 antibody lower a level of a serum biomarker for aGvHD. In some embodiments, methods of prophylaxis and/or treatment of aGvHD comprising administration of an anti-CD122 antibody lower a level of a serum biomarker for aGvHD below a threshold level.

In some embodiments, the anti-CD122 antibody administered to the subject is an antibody comprising a combination of CDRs (CDR series) as disclosed herein. In some embodiments, the anti-CD122 antibody administered to the subject is an antibody comprising a CDR series named either E1, E2, E3, E4, E5, E6, E7, E8, E9, E10, E11, E12, or E13. In some embodiments, the anti-CD122 antibody administered to the subject is an antibody comprising a CDR series named either E14, E15, E16, E17, E18, E19, E20, E21, E22, E23, E24, E25, E26, E27, or E28. In some embodiments, the anti-CD122 antibody administered to the subject is an antibody comprising a CDR series named either E29, E30, E31, E32, E33, E34, E35, E36, E37, E38, E39, or E40. In some embodiments, the anti-CD122 antibody administered to the subject is an antibody comprising a CDR series named either E41, E42, E43, E44, E45, E46, E47, E48, E49, E50, E51, E52, E53, E54, E55, or E56. In some embodiments, the anti-CD122 antibody administered to the subject is an antibody comprising a CDR series named E14. In some embodiments, the anti-CD122 antibody administered to the subject is an antibody comprising a CDR series named E15. In some embodiments, the anti-CD122 antibody administered to the subject is an antibody comprising a CDR series named E16. In some embodiments, the anti-CD122 antibody administered to the subject is an antibody comprising a CDR series named E17. In some embodiments, the anti-CD122 antibody administered to the subject is an antibody comprising a CDR series named E18. In some embodiments, the anti-CD122 antibody administered to the subject is an antibody comprising a CDR series named E19. In some embodiments, the anti-CD122 antibody administered to the subject is an antibody comprising a CDR series named E20. In some embodiments, the anti-CD122 antibody administered to the subject is an antibody comprising a CDR series named E21. In some embodiments, the anti-CD122 antibody administered to the subject is an antibody comprising a CDR series named E22. In some embodiments, the anti-CD122 antibody administered to the subject is an antibody comprising a CDR series named E23. In some embodiments, the anti-CD122 antibody administered to the subject is an antibody comprising a CDR series named E24. In some embodiments, the anti-CD122 antibody administered to the subject is an antibody comprising a CDR series named E25. In some embodiments, the anti-CD122 antibody administered to the subject is an antibody comprising a CDR series named E26. In some embodiments, the anti-CD122 antibody administered to the subject is an antibody comprising a CDR series named E27. In some embodiments, the anti-CD122 antibody administered to the subject is an antibody comprising a CDR series named E28. In some embodiments, the anti-CD122 antibody administered to the subject is an antibody comprising a CDR series named E29. In some embodiments, the anti-CD122 antibody administered to the subject is an antibody comprising a CDR series named E30. In some embodiments, the anti-CD122 antibody administered to the subject is an antibody comprising a CDR series named E31. In some embodiments, the anti-CD122 antibody administered to the subject is an antibody comprising a CDR series named E32. In some embodiments, the anti-CD122 antibody administered to the subject is an antibody comprising a CDR series named E33. In some embodiments, the anti-CD122 antibody administered to the subject is an antibody comprising a CDR series named E34. In some embodiments, the anti-CD122 antibody administered to the subject is an antibody comprising a CDR series named E35. In some embodiments, the anti-CD122 antibody administered to the subject is an antibody comprising a CDR series named E36. In some embodiments, the anti-CD122 antibody administered to the subject is an antibody comprising a CDR series named E37. In some embodiments, the anti-CD122 antibody administered to the subject is an antibody comprising a CDR series named E38. In some embodiments, the anti-CD122 antibody administered to the subject is an antibody comprising a CDR series named E39. In some embodiments, the anti-CD122 antibody administered to the subject is an antibody comprising a CDR series named E40. In some embodiments, the anti-CD122 antibody administered to the subject is an antibody comprising a CDR series named E41. In some embodiments, the anti-CD122 antibody administered to the subject is an antibody comprising a CDR series named E42. In some embodiments, the anti-CD122 antibody administered to the subject is an antibody comprising a CDR series named E43. In some embodiments, the anti-CD122 antibody administered to the subject is an antibody comprising a CDR series named E44. In some embodiments, the anti-CD122 antibody administered to the subject is an antibody comprising a CDR series named E45. In some embodiments, the anti-CD122 antibody administered to the subject is an antibody comprising a CDR series named E46. In some embodiments, the anti-CD122 antibody administered to the subject is an antibody comprising a CDR series named E47. In some embodiments, the anti-CD122 antibody administered to the subject is an antibody comprising a CDR series named E48. In some embodiments, the anti-CD122 antibody administered to the subject is an antibody comprising a CDR series named E49. In some embodiments, the anti-CD122 antibody administered to the subject is an antibody comprising a CDR series named E50. In some embodiments, the anti-CD122 antibody administered to the subject is an antibody comprising a CDR series named E51. In some embodiments, the anti-CD122 antibody administered to the subject is an antibody comprising a CDR series named E52. In some embodiments, the anti-CD122 antibody administered to the subject is an antibody comprising a CDR series named E53. In some embodiments, the anti-CD122 antibody administered to the subject is an antibody comprising a CDR series named E54. In some embodiments, the anti-CD122 antibody administered to the subject is an antibody comprising a CDR series named E55. In some embodiments, the anti-CD122 antibody administered to the subject is an antibody comprising a CDR series named E56. In some embodiments, CDR series E23 and E29-E32 reflect CDR sequences of an antibody as defined by either the Kabat, IMGT, AbM, Chothia, or Contact numbering schemes. In some embodiments, CDR series E23 and E29-E32 reflect CDR sequences of an antibody as defined by either the Kabat, IMGT, AbM, Chothia, or Contact numbering schemes, wherein the antibody comprises a VH comprising SEQ ID NO: 156 and a VL comprising SEQ ID NO: 163. In some embodiments, CDR series E23 and E29-E32 reflect CDR sequences each of distinct antibodies as defined by either the Kabat, IMGT, AbM, Chothia, or Contact numbering schemes. In some embodiments, CDR series E15 and E33-E36 reflect CDR sequences of an antibody as defined by either the Kabat, IMGT, AbM, Chothia, or Contact numbering schemes. In some embodiments, CDR series E15 and E33-E36 reflect CDR sequences of an antibody as defined by either the Kabat, IMGT, AbM, Chothia, or Contact numbering schemes, wherein the antibody comprises a VH comprising SEQ ID NO: 153 and a VL comprising SEQ ID NO: 160. In some embodiments, CDR series E15 and E33-E36 reflect CDR sequences each of distinct antibodies as defined by either the Kabat, IMGT, AbM, Chothia, or Contact numbering schemes. In some embodiments, CDR series E16 and E37-E40 reflect CDR sequences of an antibody as defined by either the Kabat, IMGT, AbM, Chothia, or Contact numbering schemes. In some embodiments, CDR series E16 and E37-E40 reflect CDR sequences of an antibody as defined by either the Kabat, IMGT, AbM, Chothia, or Contact numbering schemes, wherein the antibody comprises a VH comprising SEQ ID NO: 153 and a VL comprising SEQ ID NO: 161. In some embodiments, CDR series E16 and E37-E40 reflect CDR sequences each of distinct antibodies as defined by either the Kabat, IMGT, AbM, Chothia, or Contact numbering schemes.

In some embodiments, the anti-CD122 antibody administered to the subject is one of the antibodies described herein named G1, G2, G3, G4, G5, G6, G7, G8, G9, G10, G11, G12, G13, G14, G15, G16, G17, G18, G19, or G20. In some embodiments, Antibody 1 is an anti-CD122 antibody described herein named either G1, G2, G3, G4, G5, G6, G7, G8, G9, G10, G11, G12, G13, G14, G15, G16, G17, G18, G19, or G20. In some embodiments, the anti-CD122 antibody administered to the subject is herein named G1. In some embodiments, the anti-CD122 antibody administered to the subject is herein named G2. In some embodiments, the anti-CD122 antibody administered to the subject is herein named G3. In some embodiments, the anti-CD122 antibody administered to the subject is herein named G4. In some embodiments, the anti-CD122 antibody administered to the subject is herein named G5. In some embodiments, the anti-CD122 antibody administered to the subject is herein named G6. In some embodiments, the anti-CD122 antibody administered to the subject is herein named G7. In some embodiments, the anti-CD122 antibody administered to the subject is herein named G8. In some embodiments, the anti-CD122 antibody administered to the subject is herein named G9. In some embodiments, the anti-CD122 antibody administered to the subject is herein named G10. In some embodiments, the anti-CD122 antibody administered to the subject is herein named G11. In some embodiments, the anti-CD122 antibody administered to the subject is herein named G12. In some embodiments, the anti-CD122 antibody administered to the subject is herein named G13. In some embodiments, the anti-CD122 antibody administered to the subject is herein named G14. In some embodiments, the anti-CD122 antibody administered to the subject is herein named G15. In some embodiments, the anti-CD122 antibody administered to the subject is herein named G16. In some embodiments, the anti-CD122 antibody administered to the subject is herein named G17. In some embodiments, the anti-CD122 antibody administered to the subject is herein named G18. In some embodiments, the anti-CD122 antibody administered to the subject is herein named G19. In some embodiments, the anti-CD122 antibody administered to the subject is herein named G20. In some embodiments, the anti-CD122 antibody administered to the subject is one of the antibodies described herein named G21, G22, G23, G24, G25, G26, G27, G28, G29, G30, G31, G32, G33, G34, G35, G36, G37, G38, G39, G40, G41, G42, G43, G44, G45, G46, G47, G48, G49 or G50. In some embodiments, Antibody 2 is an anti-CD122 antibody described herein named either G41, G42, G43, G44, G45, G46, G47, G48, G49, or G50. In some embodiments, Antibody 3 is an anti-CD122 antibody described herein named either G21, G22, G23, G24, G25, G26, G27, G28, G29, or G30. In some embodiments, the anti-CD122 antibody administered to the subject is herein named G21. In some embodiments, the anti-CD122 antibody administered to the subject is herein named G22. In some embodiments, the anti-CD122 antibody administered to the subject is herein named G23. In some embodiments, the anti-CD122 antibody administered to the subject is herein named G24. In some embodiments, the anti-CD122 antibody administered to the subject is herein named G25. In some embodiments, the anti-CD122 antibody administered to the subject is herein named G26. In some embodiments, the anti-CD122 antibody administered to the subject is herein named G27. In some embodiments, the anti-CD122 antibody administered to the subject is herein named G28. In some embodiments, the anti-CD122 antibody administered to the subject is herein named G29. In some embodiments, the anti-CD122 antibody administered to the subject is herein named G30. In some embodiments, the anti-CD122 antibody administered to the subject is herein named G31. In some embodiments, the anti-CD122 antibody administered to the subject is herein named G32. In some embodiments, the anti-CD122 antibody administered to the subject is herein named G33. In some embodiments, the anti-CD122 antibody administered to the subject is herein named G34. In some embodiments, the anti-CD122 antibody administered to the subject is herein named G35. In some embodiments, the anti-CD122 antibody administered to the subject is herein named G36. In some embodiments, the anti-CD122 antibody administered to the subject is herein named G37. In some embodiments, the anti-CD122 antibody administered to the subject is herein named G38. In some embodiments, the anti-CD122 antibody administered to the subject is herein named G39. In some embodiments, the anti-CD122 antibody administered to the subject is herein named G40. In some embodiments, the anti-CD122 antibody administered to the subject is herein named G41. In some embodiments, the anti-CD122 antibody administered to the subject is herein named G42. In some embodiments, the anti-CD122 antibody administered to the subject is herein named G43. In some embodiments, the anti-CD122 antibody administered to the subject is herein named G44. In some embodiments, the anti-CD122 antibody administered to the subject is herein named G45. In some embodiments, the anti-CD122 antibody administered to the subject is herein named G46. In some embodiments, the anti-CD122 antibody administered to the subject is herein named G47. In some embodiments, the anti-CD122 antibody administered to the subject is herein named G48. In some embodiments, the anti-CD122 antibody administered to the subject is herein named G49. In some embodiments, the anti-CD122 antibody administered to the subject is herein named G50. In some embodiments, the anti-CD122 antibody administered to the subject is a mouse surrogate of one of the antibodies described herein named G21, G22, G23, G24, G25, G26, G27, G28, G29, G30, G31, G32, G33, G34, G35, G36, G37, G38, G39, G40, G41, G42, G43, G44, G45, G46, G47, G48, G49 or G50. In some embodiments, the anti-CD122 antibody administered to the subject is ChMBC7.

In some instances, patients with hematologic and lymphoid malignancies can benefit greatly from and often achieve long-term curative outcomes from allogeneic hematopoietic stem cell transplantation (alloHSCT) procedures. One of the main complications of alloHSCT is acute graft versus host disease (aGvHD). Prophylaxis of aGvHD and successful treatment of aGvHD that do present in a subject are goals of medical practitioners hoping the minimize complications arising from alloHSCT. As pro-inflammatory mediators are thought to promote aGvHD, a balanced approach to prophylaxis or treatment in which pro-inflammatory molecules and signals are minimized while still enabling a desired therapeutic benefit of graft-versus-leukemia or graft-versus-tumor would be ideal. In some embodiments, methods of prophylaxis and/or treatment of aGvHD comprising administration of an anti-CD122 antibody lower a risk of developing chronic GvHD. In some embodiments, methods of prophylaxis and/or treatment of aGvHD comprising administration of an anti-CD122 antibody prevent development of chronic GvHD.

cGvHD Treatment with an Anti-CD122 Antibody

Disclosed herein, in certain aspects, are anti-CD122 antibodies for use in methods of preventing and/or treating chronic graft versus host disease. Methods for prophylaxis of cGvHD or treatment for cGvHD comprising administration of an anti-CD122 antibody are described herein. In some embodiments, the methods of prophylaxis or treatment comprising contacting a plurality of cells in the subject with an anti-CD122 antibody. In some embodiments, the anti-CD122 antibody functions as an IL15R inhibitor in the method of treatment. In some embodiments, the anti-CD122 antibody functions as an intermediate affinity IL-βγ receptor inhibitor in the method of treatment. The intermediate affinity IL-βγ receptor is composed of CD122 beta chain and CD132 gamma chain subunits. In some embodiments, the anti-CD122 antibody functions as an IL2R inhibitor in the method of treatment. In some embodiments, the anti-CD122 antibody functions as an IL15R inhibitor and an IL2R inhibitor in the method of treatment. In some embodiments, the anti-CD122 antibody inhibits the intermediate affinity IL-βγ receptor composed of CD122 beta chain and CD132 gamma chain subunits. In some embodiments, the anti-CD122 antibody functions as an IL15R inhibitor in the method of use. In some embodiments, the anti-CD122 antibody functions as an IL2R inhibitor in the method of use. In some embodiments, the anti-CD122 antibody functions as an intermediate affinity IL-βγ receptor inhibitor in the method of use. In some embodiments, the anti-CD122 antibody functions as an IL15R inhibitor and an IL2R inhibitor in the method of use. In some embodiments, the IL15R inhibitor is an IL15Rβ inhibitor. The IL15Rβ subunit is also known as CD122. In some embodiments, the IL2R inhibitor is an IL2RP inhibitor. The IL2RP subunit is also known as CD122. In some embodiments, the anti-CD122 antibody functions as an inhibitor of the intermediate affinity IL-βγ receptor composed of CD122 beta chain and CD132 gamma chain subunits. In some embodiments, the anti-CD122 antibody functions as an inhibitor IL2 signaling and/or IL15 signaling in cells expressing the intermediate affinity IL-βγ receptor. In some embodiments, the anti-CD122 antibody interferes with IL15 binding to the IL15R and/or interferes with IL2 binding to the IL2R. In some embodiments, the anti-CD122 antibody interferes with IL15 binding to the intermediate affinity IL-βγ receptor and/or interferes with IL2 binding to the intermediate affinity IL-βγ receptor. In some instances, interfering with IL15 binding to the IL15R comprises competitive binding of the antibody to the IL15R compared to IL15 binding to the IL15R. In some instances, interfering with IL15 binding to the IL15R comprises competitive inhibition of the anti-CD122 antibody to endogenous IL15-mediated signaling. In some instances, interfering with IL2 binding to the IL2R comprises competitive binding of the anti-CD122 antibody to the IL2R compared to IL2 binding to the IL2R. In some instances, interfering with IL2 binding to the intermediate affinity IL-βγ receptor comprises competitive binding of the anti-CD122 antibody to the intermediate affinity IL-βγ receptor compared to IL2 binding to the intermediate affinity IL-βγ receptor. In some instances, interfering with IL2 binding to the IL2R comprises competitive inhibition of the anti-CD122 antibody to endogenous IL2-mediated signaling. In some instances, the anti-CD122 antibody interferes with IL2 and/or IL15 binding to the intermediate affinity IL-βγ receptor by binding to CD122 with a stronger affinity than IL2 and/or IL15 binds to intermediate affinity IL-βγ receptor. In some embodiments, the anti-CD122 antibody functions as an inhibitor of IL2-mediated signal transduction and/or IL15-mediated signal transduction in i) lymphocytes or progenitors thereof and/or ii) NK cells or progenitors thereof. In some embodiments, IL2 signaling in cells of the subject expands CD4+ helper T cells and regulatory T cells (Tregs). In some embodiments, inhibiting IL2 signaling in cells of the subject selectively affects IL2-mediated expansion of CD4+ helper T cells. In some embodiments, IL15 signaling in cells of the subject supports the development of memory T cells and NK cells. In some embodiments, inhibiting IL15 signaling in cells of the subject selectively affects IL2-mediated support of memory T cell development. In some instances in subjects having received an alloHSCT, IL15 signaling in cells of the subject is more efficient than IL2 signaling in cooperating with IL21 to boost the expansion and effector function of donor-derived CD8+ T cells. In some embodiments of methods described herein, inhibiting IL15 signaling in cells of the subject reduces expansion and effector function of donor-derived CD8+ T cells. In some embodiments, inhibiting IL15 signaling and/or IL2 signaling in cells of the subject reduces the activation and expansion of alloreactive T cells from secondary lymphoid tissues (SLTs). In some embodiments, inhibiting IL15 signaling and/or IL2 signaling in cells of the subject reduces recruitment of alloreactive T cells into cGvHD target tissues from the blood. In some embodiments, inhibiting IL15 signaling and/or IL2 signaling in cells of the subject reduces the activation and expansion of alloreactive T cells locally maintained within diseased tissue. In some instances, subjects with cGvHD avoid T cell exhaustion by continued activation and proliferation of alloreactive T cells following establishing of local sources of T cell progenitors within diseased tissue. In some embodiments of methods described herein, inhibiting IL15 signaling and/or IL2 signaling in cells of the subject reduces the activation and expansion of alloreactive T cells locally maintained within diseased tissue and results in an increased percentage of exhausted T cells compared to no inhibition of IL15 signaling and/or IL2 signaling. In some instances, a tissue-resident Tcf7-expressing (protein product is TCF-1) T cell subset population is involved in intratissue cGvHD maintenance. In some embodiments of methods described herein, inhibiting IL15 signaling and/or IL2 signaling in cells of the subject reduces activation and proliferation of tissue-resident Tcf7-expressing T cell within affected tissues of the subject. In some embodiments of methods described herein, inhibiting IL15 signaling and/or IL2 signaling in cells of the subject reduces proliferation of NK cells.

In some embodiments, the administering of the anti-CD122 antibody inhibits IL2 and/or IL15 signaling. In some embodiments, the administering of the anti-CD122 antibody inhibits IL2 signaling. In some embodiments, the administering of the anti-CD122 antibody inhibits IL15 signaling. In some embodiments, the administering of the anti-CD122 antibody inhibits IL2 signaling and IL15 signaling. In some embodiments, the inhibition of IL2 and/or IL15 signaling occurs in cell types expressing intermediate affinity IL2R or IL15R composed of beta and gamma receptor subunits. In some embodiments, the anti-CD122 antibody disrupts or diminishes an IL15/IL15Ra complex from binding to an IL15Rβ/IL15Rγ complex. In some embodiments, the anti-CD122 antibody disrupts or diminishes IL2 from binding to an IL2Rβ/IL2Rγ complex. In some embodiments, the anti-CD122 antibody disrupts or diminishes both IL2 and IL15 from binding to an intermediate affinity IL-βγ receptor. In some embodiments, the effective amount of the anti-CD122 antibody does not significantly disrupt IL2 from binding to a high affinity IL2Rα/IL2Rβ/IL2Rγ complex. In some embodiments, the effective amount of the anti-CD122 antibody does not significantly disrupt IL2 from signaling through a high affinity IL2Rα/IL2Rβ/IL2Rγ complex.

In some embodiments, the anti-CD122 antibody targets CD122 in CD122-expressing cells in a subject following administering of the antibody. In some embodiments, targeting CD122 with an anti-CD122 antibody reduces the ability of IL2 to bind to an IL2R composed of a CD122 subunit and reduces the ability to elicit an amount of IL2-mediated signal transduction. In some embodiments, targeting CD122 with an anti-CD122 antibody reduces the ability of IL15, or IL15 bound to IL15Rα, to bind to an IL15R composed of a CD122 subunit and reduces the ability to elicit an amount of IL15-mediated signal transduction. In some embodiments, targeting CD122 with an anti-CD122 antibody reduces the ability of IL2 to bind to intermediate affinity IL2R composed of beta and gamma receptor subunits. In some embodiments, targeting CD122 with an anti-CD122 antibody reduces the ability of IL15, or IL15 bound to IL15Rα, to bind to intermediate affinity IL15R composed of beta and gamma receptor subunits. In some embodiments, targeting CD122 with an anti-CD122 antibody reduces the ability of IL2 and/or IL15 from binding to intermediate affinity IL-βγ receptor and reduces the ability to elicit an extent of IL2-mediated and/or IL15-mediated signal transduction. In some embodiments, targeting CD122 with an anti-CD122 antibody reduces the ability of IL2 and/or IL15 from binding to high affinity IL-βγ receptor (composed of an alpha/beta/gamma subunit heterotrimer) and reduces the ability to elicit an extent of IL2-mediated and/or IL15-mediated signal transduction. In some embodiments, targeting CD122 with an anti-CD122 antibody reduces the ability of IL2 and/or IL15 from binding to intermediate affinity IL-βγ receptor and reduces the ability to elicit an extent of IL2-mediated and/or IL15-mediated signal transduction more effectively than acting through high affinity IL-αβγ receptor. In some embodiments, this preferential effect of targeting intermediate affinity IL-βγ receptor with an anti-CD122 antibody may be due to the specific affinity of the anti-CD122 antibody to CD122 and intermediate affinity IL-βγ receptor composed of CD122. Intermediate-affinity heterodimeric IL-βγ receptor have a K_{d}˜10⁻⁹ M for IL2 or IL15, whereas the high-affinity heterotrimer IL-αβγ receptor combinations have a K_{d}˜10⁻¹¹ M for IL2 or IL15. In some embodiments, selecting an anti-CD1122 antibody with a particular binding affinity for CD122 close to the natural affinity for IL2 or IL15 for IL-βγ receptor, or in between the natural affinity for IL2 or IL15 for IL-βγ receptor compared to the natural affinity for IL2 or IL15 for their respective IL-αβγ receptors is a means of selectively targeting particular cells or cell types. In some embodiments, cells or cell types with strong expression of intermediate affinity IL2R and/or IL15R, but lacking strong expression of high affinity IL-αβγ receptor may be selectively targeted by an anti-CD122 antibody with a K_{d}˜10⁻⁹ to 10⁻¹⁰ M. In some embodiments, cells or cell ty pes expressing high affinity IL-αβγ receptor may be less affected by an anti-CD122 antibody with a K_{d}˜10⁻⁹ to 10⁻¹⁰ M, as the antibody may not substantially interference with ligand binding to the high affinity receptor. In some embodiments, the anti-CD122 antibody administered to the subject is selected based on its binding affinity to CD122. In some embodiments, targeting CD122 with an anti-CD122 antibody reduces the ability of IL15, or IL15 bound to IL15Rα, to bind to an IL15R composed of a CD122 subunit and reduces the ability to elicit an amount of IL15-mediated signal transduction. In some embodiments, the anti-CD122 antibody binds to an epitope within the extracellular domain of CD122. In some embodiments, the anti-CD122 antibody binds to a linear epitope in CD122. In some embodiments, the anti-CD122 antibody binds to a conformational epitope in CD122. In some embodiments, the epitope overlaps with a binding region of IL2 and/or IL15 on the extracellular domain of CD122.

In some embodiments, the anti-CD122 antibody directly inhibits initiation and/or maintenance of IL2-mediated signal transduction and/or IL15-mediated signal transduction. In some embodiments, targeting CD122 with an anti-CD122 antibody can reduce the ability of IL2 to bind to an IL2R composed of a CD122 subunit and can reduce the ability to elicit an amount of IL2-mediated signal transduction. In some embodiments, targeting CD122 with an anti-CD122 antibody can reduce the ability of IL15 to bind to an IL15R composed of a CD122 subunit and can reduce the ability to elicit an amount of IL15-mediated signal transduction. In some embodiments, targeting CD122 with an anti-CD122 antibody inhibits IL2-mediated signal transduction, IL15-mediated signal transduction, or IL2-mediated signal transduction and IL15-mediated signal transduction. In some embodiments, the CD122 epitope in which the anti-CD122 antibody binds overlaps with a binding region of IL2 and/or IL15 on the extracellular domain of CD122. In some embodiments, the CD122 epitope in which the anti-CD122 antibody binds does not overlap with a binding region of IL2 and/or IL15 on the extracellular domain of CD122.

In some embodiments, the anti-CD122 antibody indirectly inhibits initiation and/or maintenance of IL2-mediated signal transduction and/or IL15-mediated signal transduction. In some embodiments, targeting CD122 with an anti-CD122 antibody does not significantly reduce the ability of IL2 to bind to an IL2R composed of a CD122 subunit, but can reduce the ability to elicit an amount of IL2-mediated signal transduction. In some embodiments, targeting CD122 with an anti-CD122 antibody does not significantly reduce the ability of IL15 to bind to an IL15R composed of a CD122 subunit, but can reduce the ability to elicit an amount of IL15-mediated signal transduction. In some embodiments, targeting CD122 with an anti-CD122 antibody inhibits IL2-mediated signal transduction, IL15-mediated signal transduction, or IL2-mediated signal transduction and IL15-mediated signal transduction. In some embodiments, targeting CD122 with an anti-CD122 antibody does not disrupt IL2 or IL15 from binding to IL2R or IL15R respectively, but prevents the IL2R complex or the IL15R complex from effectively initiating and/or maintaining IL2-mediated signal transduction or IL15-mediated signal transduction respectively. In some embodiments, the CD122 epitope in which the anti-CD122 antibody binds does not overlap with a binding region of IL2 and/or IL15 on the extracellular domain of CD122.

In some embodiments, methods described herein involve contacting one or more cells in the subject with an anti-CD122 antibody to prevent or treat cGvHD. In some embodiments, methods using the anti-CD122 antibody inhibit IL2 signaling in cells contacted with the anti-CD122 antibody. In some embodiments, methods using the anti-CD122 antibody inhibit IL15 signaling in cells contacted with the anti-CD122 antibody. In some embodiments, methods using the anti-CD122 antibody inhibit IL2 and IL15 signaling in cells contacted with the anti-CD122 antibody. In some embodiments, a plurality of cells in the subject expressing CD122 exhibit IL2 signaling inhibition after being contacted with the anti-CD122 antibody. In some embodiments, a plurality of cells in the subject expressing CD122 exhibit IL15 signaling inhibition after being contacted with the anti-CD122 antibody. In some embodiments, a plurality of cells in the subject expressing CD122 exhibit IL2 and IL15 signaling inhibition after being contacted with the anti-CD122 antibody. In some embodiments, a plurality of cells in the subject expressing CD122 are targeted by a mechanism of cell-mediated immune defense after being contacted with the anti-CD122 antibody. In some embodiments, the mechanism of cell-mediated immune defense is antibody-dependent cellular cytotoxicity (ADCC). In some embodiments, contacting a plurality of cells in the subject with the anti-CD122 antibody inhibits IL2-mediated cellular proliferation. In some embodiments, contacting a plurality of cells in the subject with the anti-CD122 antibody inhibits IL15-mediated cellular proliferation. In some embodiments, contacting a plurality of cells in the subject with the anti-CD122 antibody inhibits IL2-mediated cellular proliferation and IL15-mediated cellular proliferation. In some embodiments, the IL2-mediated cellular proliferation and/or IL15-mediated cellular proliferation affects proliferation of lymphocytes in the subject. In some embodiments, memory T cell proliferation is reduced in the subject following administration of the anti-CD122 antibody. In some embodiments, CD4+ T cell proliferation is reduced in the subject following administration of the anti-CD122 antibody. In some embodiments, CD8+ T cell proliferation is reduced in the subject following administration of the anti-CD122 antibody.

In another aspect, a method of prophylaxis for cGvHD comprises administering an effective amount of an anti-CD122 antibody described herein.

In some embodiments, methods of prophylaxis and/or treatment of cGvHD comprising administration of an anti-CD122 antibody alter a level of a serum biomarker for cGvHD. In some embodiments, methods of prophylaxis and/or treatment of cGvHD comprising administration of an anti-CD122 antibody lower a level of a serum biomarker for cGvHD. In some embodiments, methods of prophylaxis and/or treatment of cGvHD comprising administration of an anti-CD122 antibody lower a level of a serum biomarker for cGvHD below a threshold level.

In some embodiments, the anti-CD122 antibody administered to the subject is an antibody comprising a CDR series named either E1, E2, E3, E4, E5, E6, E7, E8, E9, E10, E11, E12, or E13. In some embodiments, the anti-CD122 antibody administered to the subject is an antibody comprising a CDR series named either E14, E15, E16, E17, E18, E19, E20, E21, E22, E23, E24, E25, E26, E27, or E28. In some embodiments, the anti-CD122 antibody administered to the subject is an antibody comprising a CDR series named either E29, E30, E31, E32, E33, E34, E35, E36, E37, E38, E39, or E40. In some embodiments, the anti-CD122 antibody administered to the subject is an antibody comprising a CDR series named either E41, E42, E43, E44, E45, E46, E47, E48, E49, E50, E51, E52, E53, E54, E55, or E56. In some embodiments, CDR series E23 and E29-E32 reflect CDR sequences of an antibody as defined by either the Kabat, IMGT, AbM, Chothia, or Contact numbering schemes. In some embodiments, CDR series E23 and E29-E32 reflect CDR sequences of an antibody as defined by either the Kabat, IMGT, AbM, Chothia, or Contact numbering schemes, wherein the antibody comprises a VH comprising SEQ ID NO: 156 and a VL comprising SEQ ID NO: 163. In some embodiments, CDR series E23 and E29-E32 reflect CDR sequences each of distinct antibodies as defined by either the Kabat, IMGT, AbM, Chothia, or Contact numbering schemes. In some embodiments, CDR series E15 and E33-E36 reflect CDR sequences of an antibody as defined by either the Kabat, IMGT, AbM, Chothia, or Contact numbering schemes. In some embodiments, CDR series E15 and E33-E36 reflect CDR sequences of an antibody as defined by either the Kabat, IMGT, AbM, Chothia, or Contact numbering schemes, wherein the antibody comprises a VH comprising SEQ ID NO: 153 and a VL comprising SEQ ID NO: 160. In some embodiments, CDR series E15 and E33-E36 reflect CDR sequences each of distinct antibodies as defined by either the Kabat, IMGT, AbM, Chothia, or Contact numbering schemes. In some embodiments, CDR series E16 and E37-E40 reflect CDR sequences of an antibody as defined by either the Kabat, IMGT, AbM, Chothia, or Contact numbering schemes. In some embodiments, CDR series E16 and E37-E40 reflect CDR sequences of an antibody as defined by either the Kabat, IMGT, AbM, Chothia, or Contact numbering schemes, wherein the antibody comprises a VH comprising SEQ ID NO: 153 and a VL comprising SEQ ID NO: 161. In some embodiments, CDR series E16 and E37-E40 reflect CDR sequences each of distinct antibodies as defined by either the Kabat, IMGT, AbM, Chothia, or Contact numbering schemes. In some embodiments, the anti-CD122 antibody administered to the subject is an antibody comprising a CDR series named E14. In some embodiments, the anti-CD122 antibody administered to the subject is an antibody comprising a CDR series named E15. In some embodiments, the anti-CD122 antibody administered to the subject is an antibody comprising a CDR series named E16. In some embodiments, the anti-CD122 antibody administered to the subject is an antibody comprising a CDR series named E17. In some embodiments, the anti-CD122 antibody administered to the subject is an antibody comprising a CDR series named E18. In some embodiments, the anti-CD122 antibody administered to the subject is an antibody comprising a CDR series named E19. In some embodiments, the anti-CD122 antibody administered to the subject is an antibody comprising a CDR series named E20. In some embodiments, the anti-CD122 antibody administered to the subject is an antibody comprising a CDR series named E21. In some embodiments, the anti-CD122 antibody administered to the subject is an antibody comprising a CDR series named E22. In some embodiments, the anti-CD122 antibody administered to the subject is an antibody comprising a CDR series named E23. In some embodiments, the anti-CD122 antibody administered to the subject is an antibody comprising a CDR series named E24. In some embodiments, the anti-CD122 antibody administered to the subject is an antibody comprising a CDR series named E25. In some embodiments, the anti-CD122 antibody administered to the subject is an antibody comprising a CDR series named E26. In some embodiments, the anti-CD122 antibody administered to the subject is an antibody comprising a CDR series named E27. In some embodiments, the anti-CD122 antibody administered to the subject is an antibody comprising a CDR series named E28. In some embodiments, the anti-CD122 antibody administered to the subject is an antibody comprising a CDR series named E29. In some embodiments, the anti-CD122 antibody administered to the subject is an antibody comprising a CDR series named E30. In some embodiments, the anti-CD122 antibody administered to the subject is an antibody comprising a CDR series named E31. In some embodiments, the anti-CD122 antibody administered to the subject is an antibody comprising a CDR series named E32. In some embodiments, the anti-CD122 antibody administered to the subject is an antibody comprising a CDR series named E33. In some embodiments, the anti-CD122 antibody administered to the subject is an antibody comprising a CDR series named E34. In some embodiments, the anti-CD122 antibody administered to the subject is an antibody comprising a CDR series named E35. In some embodiments, the anti-CD122 antibody administered to the subject is an antibody comprising a CDR series named E36. In some embodiments, the anti-CD122 antibody administered to the subject is an antibody comprising a CDR series named E37. In some embodiments, the anti-CD122 antibody administered to the subject is an antibody comprising a CDR series named E38. In some embodiments, the anti-CD122 antibody administered to the subject is an antibody comprising a CDR series named E39. In some embodiments, the anti-CD122 antibody administered to the subject is an antibody comprising a CDR series named E40. In some embodiments, the anti-CD122 antibody administered to the subject is an antibody comprising a CDR series named E41. In some embodiments, the anti-CD122 antibody administered to the subject is an antibody comprising a CDR series named E42. In some embodiments, the anti-CD122 antibody administered to the subject is an antibody comprising a CDR series named E43. In some embodiments, the anti-CD122 antibody administered to the subject is an antibody comprising a CDR series named E44. In some embodiments, the anti-CD122 antibody administered to the subject is an antibody comprising a CDR series named E45. In some embodiments, the anti-CD122 antibody administered to the subject is an antibody comprising a CDR series named E46. In some embodiments, the anti-CD122 antibody administered to the subject is an antibody comprising a CDR series named E47. In some embodiments, the anti-CD122 antibody administered to the subject is an antibody comprising a CDR series named E48. In some embodiments, the anti-CD122 antibody administered to the subject is an antibody comprising a CDR series named E49. In some embodiments, the anti-CD122 antibody administered to the subject is an antibody comprising a CDR series named E50. In some embodiments, the anti-CD122 antibody administered to the subject is an antibody comprising a CDR series named E51. In some embodiments, the anti-CD122 antibody administered to the subject is an antibody comprising a CDR series named E52. In some embodiments, the anti-CD122 antibody administered to the subject is an antibody comprising a CDR series named E53. In some embodiments, the anti-CD122 antibody administered to the subject is an antibody comprising a CDR series named E54. In some embodiments, the anti-CD122 antibody administered to the subject is an antibody comprising a CDR series named E55. In some embodiments, the anti-CD122 antibody administered to the subject is an antibody comprising a CDR series named E56.

In some embodiments, the anti-CD122 antibody administered to the subject is one of the antibodies described herein named G1, G2, G3, G4, G5, G6, G7, G8, G9, G10, G11, G12, G13, G14, G15, G16, G17, G18, G19, or G20. In some embodiments, Antibody 1 is an anti-CD122 antibodies described herein named either G1, G2, G3, G4, G5, G6, G7, G8, G9, G10, G11, G12, G13, G14, G15, G16, G17, G18, G19, or G20. In some embodiments, the anti-CD122 antibody administered to the subject is herein named G1. In some embodiments, the anti-CD122 antibody administered to the subject is herein named G2. In some embodiments, the anti-CD122 antibody administered to the subject is herein named G3. In some embodiments, the anti-CD122 antibody administered to the subject is herein named G4. In some embodiments, the anti-CD122 antibody administered to the subject is herein named G5. In some embodiments, the anti-CD122 antibody administered to the subject is herein named G6. In some embodiments, the anti-CD122 antibody administered to the subject is herein named G7. In some embodiments, the anti-CD122 antibody administered to the subject is herein named G8. In some embodiments, the anti-CD122 antibody administered to the subject is herein named G9. In some embodiments, the anti-CD122 antibody administered to the subject is herein named G10. In some embodiments, the anti-CD122 antibody administered to the subject is herein named G11. In some embodiments, the anti-CD122 antibody administered to the subject is herein named G12. In some embodiments, the anti-CD122 antibody administered to the subject is herein named G13. In some embodiments, the anti-CD122 antibody administered to the subject is herein named G14. In some embodiments, the anti-CD122 antibody administered to the subject is herein named G15. In some embodiments, the anti-CD122 antibody administered to the subject is herein named G16. In some embodiments, the anti-CD122 antibody administered to the subject is herein named G17. In some embodiments, the anti-CD122 antibody administered to the subject is herein named G18. In some embodiments, the anti-CD122 antibody administered to the subject is herein named G19. In some embodiments, the anti-CD122 antibody administered to the subject is herein named G20. In some embodiments, the anti-CD122 antibody administered to the subject is one of the antibodies described herein named G21, G22, G23, G24, G25, G26, G27, G28, G29, G30, G31, G32, G33, G34, G35, G36, G37, G38, G39, G40, G41, G42, G43, G44, G45, G46, G47, G48, G49 or G50. In some embodiments, Antibody 2 is an anti-CD122 antibody described herein named either G41, G42, G43, G44, G45, G46, G47, G48, G49, or G50. In some embodiments, Antibody 3 is an anti-CD122 antibody described herein named either G21, G22, G23, G24, G25, G26, G27, G28, G29, or G30. In some embodiments, the anti-CD122 antibody administered to the subject is herein named G21. In some embodiments, the anti-CD122 antibody administered to the subject is herein named G22. In some embodiments, the anti-CD122 antibody administered to the subject is herein named G23. In some embodiments, the anti-CD122 antibody administered to the subject is herein named G24. In some embodiments, the anti-CD122 antibody administered to the subject is herein named G25. In some embodiments, the anti-CD122 antibody administered to the subject is herein named G26. In some embodiments, the anti-CD122 antibody administered to the subject is herein named G27. In some embodiments, the anti-CD122 antibody administered to the subject is herein named G28. In some embodiments, the anti-CD122 antibody administered to the subject is herein named G29. In some embodiments, the anti-CD122 antibody administered to the subject is herein named G30. In some embodiments, the anti-CD122 antibody administered to the subject is herein named G31. In some embodiments, the anti-CD122 antibody administered to the subject is herein named G32. In some embodiments, the anti-CD122 antibody administered to the subject is herein named G33. In some embodiments, the anti-CD122 antibody administered to the subject is herein named G34. In some embodiments, the anti-CD122 antibody administered to the subject is herein named G35. In some embodiments, the anti-CD122 antibody administered to the subject is herein named G36. In some embodiments, the anti-CD122 antibody administered to the subject is herein named G37. In some embodiments, the anti-CD122 antibody administered to the subject is herein named G38. In some embodiments, the anti-CD122 antibody administered to the subject is herein named G39. In some embodiments, the anti-CD122 antibody administered to the subject is herein named G40. In some embodiments, the anti-CD122 antibody administered to the subject is herein named G41. In some embodiments, the anti-CD122 antibody administered to the subject is herein named G42. In some embodiments, the anti-CD122 antibody administered to the subject is herein named G43. In some embodiments, the anti-CD122 antibody administered to the subject is herein named G44. In some embodiments, the anti-CD122 antibody administered to the subject is herein named G45. In some embodiments, the anti-CD122 antibody administered to the subject is herein named G46. In some embodiments, the anti-CD122 antibody administered to the subject is herein named G47. In some embodiments, the anti-CD122 antibody administered to the subject is herein named G48. In some embodiments, the anti-CD122 antibody administered to the subject is herein named G49. In some embodiments, the anti-CD122 antibody administered to the subject is herein named G50. In some embodiments, the anti-CD122 antibody administered to the subject is a mouse surrogate of one of the antibodies described herein named G21, G22, G23, G24, G25, G26, G27, G28, G29, G30, G31, G32, G33, G34, G35, G36, G37, G38, G39, G40, G41, G42, G43, G44, G45, G46, G47, G48, G49 or G50. In some embodiments, the anti-CD122 antibody administered to the subject is ChMBC7.

In some instances, patients with hematologic and lymphoid malignancies can benefit greatly from and often achieve long-term curative outcomes from allogeneic hematopoietic stem cell transplantation (alloHSCT) procedures. One of the main complications of alloHSCT is chronic graft versus host disease (cGvHD). Prophylaxis of cGvHD and successful treatment of cGvHD that do present in a subject are goals of medical practitioners hoping the minimize complications arising from alloHSCT. As pro-inflammatory mediators are thought to promote cGvHD, a balanced approach to prophylaxis or treatment in which pro-inflammatory molecules and signals are minimized while still enabling a desired therapeutic benefit of graft-versus-leukemia or graft-versus-tumor would be ideal.

### Anti-CD122 Antibodies

Provided herein are antibodies that bind to CD122. In some instances, the antibodies that bind to CD122 are monoclonal antibodies. In certain aspects, disclosed herein is an anti-CD122 antibody. In some instances, the anti-CD122 antibody specifically binds to mammalian CD122. In some instances, the anti-CD122 antibody specifically binds to a human CD122. In some instances, the anti-CD122 antibody specifically binds to an extracellular portion of CD122. In some instances, the anti-CD122 antibody specifically binds to an extracellular portion of human CD122. In some instances, the anti-CD122 antibody is made of chimeric amino acid sequences some of which are murine-derived and some of which are human-derived. In some instances, the anti-CD122 antibody is made with complementarity-determining regions (CDRs) that have been incorporated into an antibody scaffold. In some instances, the anti-CD122 antibody is made with complementarity-determining regions (CDRs) incorporated into a human antibody variable region framework. In some instances, the human antibody variable region framework has been sequence-optimized to retain CD122 affinity with the engrafted mouse CDR sequences. In some instances, the anti-CD122 antibody is a humanized antibody. In some instances, the anti-CD122 antibody is a human antibody.

In some embodiments, the anti-CD122 antibody comprises i) a heavy chain comprising a variable heavy chain (VH) domain and ii) a light chain comprising a variable light chain (VL) domain. In some embodiments, VH domain comprises heavy chain CDR1 (HCDR1) sequence comprising a sequence selected from SEQ ID NOs: 1-11, heavy chain CDR2 (HCDR2) sequence comprising a sequence selected from SEQ ID NOs: 12-23, and heavy chain CDR3 (HCDR3) sequence comprising a sequence selected from SEQ ID NOs: 24-36. In some embodiments, VL domain comprises light chain CDR1 (LCDR1) sequence comprising a sequence selected from SEQ ID NOs: 37-47, light chain CDR2 (LCDR2) sequence comprising a sequence selected GTS, TTS, YTS, WAS, KAS, GAT, YAS or STS, and light chain CDR3 (LCDR3) sequence comprising a sequence selected from SEQ ID NOs: 56-67. In some embodiments, the VH region of the anti-CD122 antibody comprises HCDR1, HCDR2, and HCDR3 sequences selected from Table 1.

TABLE 1
HCDR Sequences

| SEQ ID NO: | HCDR1 Sequence |
|---|---|
| 1 | GFSLTSYG |
| 2 | GYTFTSYW |
| 3 | GSTFNRYW |
| 4 | GFTFTDYN |
| 5 | GYSFTAYT |
| 6 | GFNIKDDY |
| 7 | GYTFTSHW |
| 8 | GFTFSTFA |
| 9 | GFTFTDHT |
| 10 | GFSLTSYD |
| 11 | GYTFTAYW |
| SEQ ID NO: | HCDR2 Sequence |
| 12 | MWGGGST |
| 13 | IYPGRGST |
| 14 | ILPGSGNT |
| 15 | INPNNGRS |
| 16 | INPYNGYA |
| 17 | IDPENGDT |
| 18 | IYPGSGNT |
| 19 | ITGDGGTYT |
| 20 | IYPRDGYT |
| 21 | IWGGGGST |
| 22 | IWTGGGT |
| 23 | IDPNSGYT |
| SEQ ID NO: | HCDR3 Sequence |
| 24 | ARRTYSDSYYYEMDY |
| 25 | ARELGGFAY |
| 26 | ARLDYYGSRYYFDY |
| 27 | AREDWEGFYAMDY |
| 28 | ARVGYYFDY |
| 29 | TGYFDY |
| 30 | ARERGGFDY |
| 31 | ARHSVSSWFAY |
| 32 | ARPTSLLRFPY |
| 33 | ARHNYDGYYYSLDY |
| 34 | ARHNYDNYYYTLDY |
| 35 | VRDLFPYAMDY |
| 36 | ARGHFGYDDS |

In some embodiments, the VH region comprises HCDR1 sequence comprising SEQ ID NO: 1; HCDR2 sequence comprising SEQ ID NO: 12; and HCDR3 sequence comprising SEQ ID NO: 24. In some embodiments, the VH region comprises HCDR1 sequence comprising SEQ ID NO: 2; HCDR2 sequence comprising SEQ ID NO: 13; and HCDR3 sequence comprising SEQ ID NO: 25. In some embodiments, the VH region comprises HCDR1 sequence comprising SEQ ID NO: 3; HCDR2 sequence comprising SEQ ID NO: 14; and HCDR3 sequence comprising SEQ ID NO: 26. In some embodiments, the VH region comprises HCDR1 sequence comprising SEQ ID NO: 4; HCDR2 sequence comprising SEQ ID NO: 15; and HCDR3 sequence comprising SEQ ID NO: 27. In some embodiments, the VH region comprises HCDR1 sequence comprising SEQ ID NO: 5; HCDR2 sequence comprising SEQ ID NO: 16; and HCDR3 sequence comprising SEQ ID NO: 28. In some embodiments, the VH region comprises HCDR1 sequence comprising SEQ ID NO: 6; HCDR2 sequence comprising SEQ ID NO: 17; and HCDR3 sequence comprising SEQ ID NO: 29. In some embodiments, the VH region comprises HCDR1 sequence comprising SEQ ID NO: 7; HCDR2 sequence comprising SEQ ID NO: 18; and HCDR3 sequence comprising SEQ ID NO: 30. In some embodiments, the VH region comprises HCDR1 sequence comprising SEQ ID NO: 8; HCDR2 sequence comprising SEQ ID NO: 19; and HCDR3 sequence comprising SEQ ID NO: 31. In some embodiments, the VH region comprises HCDR1 sequence comprising SEQ ID NO: 9; HCDR2 sequence comprising SEQ ID NO: 20; and HCDR3 sequence comprising SEQ ID NO: 32. In some embodiments, the VH region comprises HCDR1 sequence comprising SEQ ID NO: 1; HCDR2 sequence comprising SEQ ID NO: 21; and HCDR3 sequence comprising SEQ ID NO: 33. In some embodiments, the VH region comprises HCDR1 sequence comprising SEQ ID NO: 1; HCDR2 sequence comprising SEQ ID NO: 21; and HCDR3 sequence comprising SEQ ID NO: 34. In some embodiments, the VH region comprises HCDR1 sequence comprising SEQ ID NO: 10; HCDR2 sequence comprising SEQ ID NO: 22; and HCDR3 sequence comprising SEQ ID NO: 35. In some embodiments, the VH region comprises HCDR1 sequence comprising SEQ ID NO: 11; HCDR2 sequence comprising SEQ ID NO: 23; and HCDR3 sequence comprising SEQ ID NO: 36.

In some embodiments, the VL region of the anti-CD122 antibody comprises LCDR1, LCDR2, and LCDR3 sequences selected from Table 2.

TABLE 2
LCDR Sequences

| SEQ ID NO: | LCDR1 Sequence |
|---|---|
| 37 | SSVSSRY |
| 38 | SYVSSSY |
| 39 | QDIRNY |
| 40 | QNVDTD |
| 41 | SSVSY |
| 42 | QNINLW |
| 43 | QDISNF |
| 44 | SSVNY |
| 45 | EDIDSY |
| 46 | QTIGTS |
| 47 | SRVSY |
| SEQ ID NO: | LCDR2 Sequence |
| — | GTS |
| — | TTS |
| — | YTS |
| — | WAS |
| — | KAS |
| — | GAT |
| — | YAS |
| — | STS |
| SEQ ID NO: | LCDR3 Sequence |
| 56 | QQYHSDPLT |
| 57 | HQYHLSPWT |
| 58 | QQGDPLPPT |
| 59 | EQYSSYPYT |
| 60 | QQFTSSPYT |
| 61 | LQGQSYPWT |
| 62 | QQDNNHPYT |
| 63 | QQFTSSPWT |
| 64 | QHYYSIPYT |
| 65 | QQYHGDPLT |
| 66 | QQTNSWPLT |
| 67 | QQRSSYPLT |

In some embodiments, the VL region comprises LCDR1 sequence comprising SEQ ID NO: 37; LCDR2 sequence comprising GTS; and LCDR3 sequence comprising SEQ ID NO: 56. In some embodiments, the VL region comprises LCDR1 sequence comprising SEQ ID NO: 38; LCDR2 sequence comprising TTS; and LCDR3 sequence comprising SEQ ID NO: 57. In some embodiments, the VL region comprises LCDR1 sequence comprising SEQ ID NO: 39; LCDR2 sequence comprising YTS; and LCDR3 sequence comprising SEQ ID NO: 58. In some embodiments, the VL region comprises LCDR1 sequence comprising SEQ ID NO: 40; LCDR2 sequence comprising WAS; and LCDR3 sequence comprising SEQ ID NO: 59. In some embodiments, the VL region comprises LCDR1 sequence comprising SEQ ID NO: 41; LCDR2 sequence comprising YTS; and LCDR3 sequence comprising SEQ ID NO: 60. In some embodiments, the VL region comprises LCDR1 sequence comprising SEQ ID NO: 42; LCDR2 sequence comprising KAS; and LCDR3 sequence comprising SEQ ID NO: 61. In some embodiments, the VL region comprises LCDR1 sequence comprising SEQ ID NO: 43; LCDR2 sequence comprising YTS; and LCDR3 sequence comprising SEQ ID NO: 62. In some embodiments, the VL region comprises LCDR1 sequence comprising SEQ ID NO: 44; LCDR2 sequence comprising YTS; and LCDR3 sequence comprising SEQ ID NO: 63. In some embodiments, the VL region comprises LCDR1 sequence comprising SEQ ID NO: 45; LCDR2 sequence comprising GAT; and LCDR3 sequence comprising SEQ ID NO: 64. In some embodiments, the VL region comprises LCDR1 sequence comprising SEQ ID NO: 37; LCDR2 sequence comprising GTS; and LCDR3 sequence comprising SEQ ID NO: 65. In some embodiments, the VL region comprises LCDR1 sequence comprising SEQ ID NO: 46; LCDR2 sequence comprising YAS; and LCDR3 sequence comprising SEQ ID NO: 66. In some embodiments, the VL region comprises LCDR1 sequence comprising SEQ ID NO: 47; LCDR2 sequence comprising STS; and LCDR3 sequence comprising SEQ ID NO: 67.

In some embodiments, the anti-CD122 antibody comprises a VH region and a VL region, in which the VH region comprises HCDR1 sequence comprising SEQ ID NO: 1; HCDR2 sequence comprising SEQ ID NO: 12; and HCDR3 sequence comprising SEQ ID NO: 24 and in which the VL region comprises LCDR1 sequence comprising SEQ ID NO: 37; LCDR2 sequence comprising GTS; and LCDR3 sequence comprising SEQ ID NO: 56. In some embodiments, the anti-CD122 antibody comprises a VH region and a VL region, in which the VH region comprises HCDR1 sequence comprising SEQ ID NO: 2; HCDR2 sequence comprising SEQ ID NO: 13; and HCDR3 sequence comprising SEQ ID NO: 25 and in which the VL region comprises LCDR1 sequence comprising SEQ ID NO: 38; LCDR2 sequence comprising TTS; and LCDR3 sequence comprising SEQ ID NO: 57. In some embodiments, the anti-CD122 antibody comprises a VH region and a VL region, in which the VH region comprises HCDR1 sequence comprising SEQ ID NO: 3; HCDR2 sequence comprising SEQ ID NO: 14; and HCDR3 sequence comprising SEQ ID NO: 26 and in which the VL region comprises LCDR1 sequence comprising SEQ ID NO: 39; LCDR2 sequence comprising YTS; and LCDR3 sequence comprising SEQ ID NO: 58. In some embodiments, the anti-CD122 antibody comprises a VH region and a VL region, in which the VH region comprises HCDR1 sequence comprising SEQ ID NO: 4; HCDR2 sequence comprising SEQ ID NO: 15; and HCDR3 sequence comprising SEQ ID NO: 27 and in which the VL region LCDR1 sequence comprising SEQ ID NO: 40; LCDR2 sequence comprising WAS; and LCDR3 sequence comprising SEQ ID NO: 59. In some embodiments, the anti-CD122 antibody comprises a VH region and a VL region, in which the VH region comprises HCDR1 sequence comprising SEQ ID NO: 5; HCDR2 sequence comprising SEQ ID NO: 16; and HCDR3 sequence comprising SEQ ID NO: 28 and in which the VL region comprises LCDR1 sequence comprising SEQ ID NO: 41; LCDR2 sequence comprising YTS; and LCDR3 sequence comprising SEQ ID NO: 60. In some embodiments, the anti-CD122 antibody comprises a VH region and a VL region, in which the VH region comprises HCDR1 sequence comprising SEQ ID NO: 6; HCDR2 sequence comprising SEQ ID NO: 17; and HCDR3 sequence comprising SEQ ID NO: 29 and in which the VL region comprises LCDR1 sequence comprising SEQ ID NO: 42; LCDR2 sequence comprising KAS; and LCDR3 sequence comprising SEQ ID NO: 61. In some embodiments, the anti-CD122 antibody comprises a VH region and a VL region, in which the VH region comprises HCDR1 sequence comprising SEQ ID NO: 7; HCDR2 sequence comprising SEQ ID NO: 18; and HCDR3 sequence comprising SEQ ID NO: 30 and in which the VL region comprises LCDR1 sequence comprising SEQ ID NO: 43; LCDR2 sequence comprising YTS; and LCDR3 sequence comprising SEQ ID NO: 62. In some embodiments, the anti-CD122 antibody comprises a VH region and a VL region, in which the VH region comprises HCDR1 sequence comprising SEQ ID NO: 8; HCDR2 sequence comprising SEQ ID NO: 19; and HCDR3 sequence comprising SEQ ID NO: 31 and in which the VL region comprises LCDR1 sequence comprising SEQ ID NO: 44; LCDR2 sequence comprising YTS; and LCDR3 sequence comprising SEQ ID NO: 63. In some embodiments, the anti-CD122 antibody comprises a VH region and a VL region, in which the VH region comprises HCDR1 sequence comprising SEQ ID NO: 9; HCDR2 sequence comprising SEQ ID NO: 20; and HCDR3 sequence comprising SEQ ID NO: 32 and in which the VL region comprises LCDR1 sequence comprising SEQ ID NO: 45; LCDR2 sequence comprising GAT; and LCDR3 sequence comprising SEQ ID NO: 64. In some embodiments, the anti-CD122 antibody comprises a VH region and a VL region, in which the VH region comprises HCDR1 sequence comprising SEQ ID NO: 1; HCDR2 sequence comprising SEQ ID NO: 21; and HCDR3 sequence comprising SEQ ID NO: 33 and in which the VL region comprises LCDR1 sequence comprising SEQ ID NO: 37; LCDR2 sequence comprising GTS; and LCDR3 sequence comprising SEQ ID NO: 65. In some embodiments, the anti-CD122 antibody comprises a VH region and a VL region, in which the VH region comprises HCDR1 sequence comprising SEQ ID NO: 1; HCDR2 sequence comprising SEQ ID NO: 21; and HCDR3 sequence comprising SEQ ID NO: 34 and in which the VL region comprises LCDR1 sequence comprising SEQ ID NO: 37; LCDR2 sequence comprising GTS; and LCDR3 sequence comprising SEQ ID NO: 65. In some embodiments, the anti-CD122 antibody comprises a VH region and a VL region, in which the VH region comprises HCDR1 sequence comprising SEQ ID NO: 10; HCDR2 sequence comprising SEQ ID NO: 22; and HCDR3 sequence comprising SEQ ID NO: 35 and in which the VL region comprises LCDR1 sequence comprising SEQ ID NO: 46; LCDR2 sequence comprising YAS; and LCDR3 sequence comprising SEQ ID NO: 66. In some embodiments, the anti-CD122 antibody comprises a VH region and a VL region, in which the VH region comprises HCDR1 sequence comprising SEQ ID NO: 11; HCDR2 sequence comprising SEQ ID NO: 23; and HCDR3 sequence comprising SEQ ID NO 36 and in which the VL region comprises LCDR1 sequence comprising SEQ ID NOS 47 LCDR2 sequence comprising STS; and LCDR3 sequence comprising SEQ ID NO: 67.

In some embodiments, the anti-CD122 antibody comprises a series of CDR sequences. In some embodiments, the series of CDR sequences comprises an HCDR1 sequence, an HCDR2 sequence, an HCDR3 sequence, an LCDR1 sequence, an LCDR2 sequence, and an LCDR3 sequence. The series of CDR sequences for each anti-CD122 antibody described herein is listed in Table 3. The names for the series of CDR sequences are E1-E13.

TABLE 3
Series of CDR sequences for anti-CD122 antibodies

| Name | HCDR1 | HCDR2 | HCDR3 | LCDR1 | LCDR2 | LCDR3 |
|---|---|---|---|---|---|---|
| E1 | SEQ ID | SEQ ID | SEQ ID | SEQ ID | GTS | SEQ ID |
| | NO: 1 | NO: 12 | NO: 24 | NO: 37 | | NO: 56 |
| E2 | SEQ ID | SEQ ID | SEQ ID | SEQ ID | TTS | SEQ ID |
| | NO: 2 | NO: 13 | NO: 25 | NO: 38 | | NO: 57 |
| E3 | SEQ ID | SEQ ID | SEQ ID | SEQ ID | YTS | SEQ ID |
| | NO: 3 | NO: 14 | NO: 26 | NO: 39 | | NO: 58 |
| E4 | SEQ ID | SEQ ID | SEQ ID | SEQ ID | WAS | SEQ ID |
| | NO: 4 | NO: 15 | NO: 27 | NO: 40 | | NO: 59 |
| E5 | SEQ ID | SEQ ID | SEQ ID | SEQ ID | YTS | SEQ ID |
| | NO: 5 | NO: 16 | NO: 28 | NO: 41 | | NO: 60 |
| E6 | SEQ ID | SEQ ID | SEQ ID | SEQ ID | KAS | SEQ ID |
| | NO: 6 | NO: 17 | NO: 29 | NO: 42 | | NO: 61 |
| E7 | SEQ ID | SEQ ID | SEQ ID | SEQ ID | YTS | SEQ ID |
| | NO: 7 | NO: 18 | NO: 30 | NO: 43 | | NO: 62 |
| E8 | SEQ ID | SEQ ID | SEQ ID | SEQ ID | YTS | SEQ ID |
| | NO: 8 | NO: 19 | NO: 31 | NO: 44 | | NO: 63 |
| E9 | SEQ ID | SEQ ID | SEQ ID | SEQ ID | GAT | SEQ ID |
| | NO: 9 | NO: 20 | NO: 32 | NO: 45 | | NO: 64 |
| E10 | SEQ ID | SEQ ID | SEQ ID | SEQ ID | GTS | SEQ ID |
| | NO: 1 | NO: 21 | NO: 33 | NO: 37 | | NO: 65 |
| E11 | SEQ ID | SEQ ID | SEQ ID | SEQ ID | GTS | SEQ ID |
| | NO: 1 | NO: 21 | NO: 34 | NO: 37 | | NO: 65 |
| E12 | SEQ ID | SEQ ID | SEQ ID | SEQ ID | YAS | SEQ ID |
| | NO: 10 | NO: 22 | NO: 35 | NO: 46 | | NO: 66 |
| E13 | SEQ ID | SEQ ID | SEQ ID | SEQ ID | STS | SEQ ID |
| | NO: 11 | NO: 23 | NO: 36 | NO: 47 | | NO: 67 |

In some embodiments, the anti-CD122 antibody comprises a framework for grafting CDRs from another animal species. In some embodiments, CDRs from a mammalian antibody are grafted onto a human framework sequence. In some embodiments, CDRs from a mouse antibody are grafted onto a human framework sequence. In some embodiments, the human framework sequence forms part of the VH region of an antibody. In some embodiments, sequences from HCDR1, HCDR2, and HCDR3 are grafted into a human framework sequence.

In some embodiments, an anti-CD122 antibody described herein comprises an IgG framework, an IgA framework, an IgE framework, or an IgM framework. In some instances, the anti-CD122 antibody comprises an IgG framework (e.g., IgG1, IgG2, IgG3, or IgG4). In some cases, the anti-CD122 antibody comprises an IgG1 framework. In some cases, the anti-CD122 antibody comprises an IgG2 (e.g., an IgG2a or IgG2b) framework. In some cases, the anti-CD122 antibody comprises an IgG2a framework. In some cases, the anti-CD122 antibody comprises an IgG2b framework. In some cases, the anti-CD122 antibody comprises an IgG3 framework. In some cases, the anti-CD122 antibody comprises an IgG4 framework. In some embodiments, the human framework sequence is inserted into an IgG backbone to form a heavy chain sequence. In some embodiments, the IgG backbone is an IgG1 backbone, an IgG2 backbone, an IgG3 backbone, or an IgG4 backbone. In some embodiments, the IgG framework comprises a human IgG heavy chain framework sequence. In some embodiments, the human IgG heavy chain framework sequence has anti-CD122 HCDR sequences grafted into it. In some embodiments, the human IgG heavy chain framework sequence with anti-CD122 HCDR sequences grafted into it is analyzed and modeled for monoclonal antibody 3D structure to identify key amino acid positions supporting CDR loop structure. In some embodiments, key amino acid positions are identified in the human IgG heavy chain framework sequence that if reverted back to a mouse framework sequence from a mouse anti-CD122 antibody, will restore affinity to human CD122 in the context of a humanized antibody using a human IgG heavy chain framework sequence that has mouse anti-CD122 CDR sequences grafted into it. In some embodiments, those key amino acid positions are mutated in the human IgG heavy chain framework sequences back to the mouse sequences and are called back mutations. In some embodiments, the human IgG heavy chain framework sequences with mouse anti-CD122 CDR sequences grafted into it and back mutations incorporated into the framework sequence are used in a human IgG backbone to create a humanized IgG heavy chain sequence. In some embodiments, the humanized IgG heavy chain sequence is used to create an anti-CD122 antibody. In some embodiments, HCDR sequences from one of series E1-E13 are grafted into a framework sequence to create a heavy chain variable domain sequence.

In some embodiments, the anti-CD122 antibody comprises a framework for grafting CDRs from another animal species. In some embodiments, CDRs from a mammalian antibody are grafted onto a human framework sequence. In some embodiments, CDRs from a mouse antibody are grafted onto a human framework sequence. In some embodiments, the human framework sequence forms part of the VL region of an antibody. In some embodiments, sequences from LCDR1, LCDR2, and LCDR3 are grafted into a human framework sequence.

In some embodiments, an anti-CD122 antibody described herein comprises a lambda or a kappa framework. In some instances, the anti-CD122 antibody comprises a kappa framework that has CDR sequences grafted into it. In some instances, the anti-CD122 antibody comprises a kappa framework that has LCDR1, LCDR2, and LCDR3 sequences grafted into it. In some embodiments, the kappa framework forms part of a light chain. In some embodiments, the kappa framework forming part of a light chain is paired with a heavy chain described herein. In some embodiments, the kappa framework is a human kappa light chain framework. In some embodiments, the human kappa light chain framework comprises a human kappa light chain framework sequence. In some embodiments, the human kappa light chain framework sequence has anti-CD122 LCDR sequences grafted into it. In some embodiments, the human kappa light chain framework sequence with anti-CD122 LCDR sequences grafted into it is analyzed and modeled for monoclonal antibody 3D structure to identify key amino acid positions supporting CDR loop structure. In some embodiments, key amino acid positions are identified in the human kappa light chain framework sequence that if reverted back to a mouse framework sequence from a mouse anti-CD122 antibody, will restore affinity to human CD122 in the context of a humanized antibody using a human kappa light chain framework sequence that has mouse anti-CD122 CDR sequences grafted into it. In some embodiments, those key amino acid positions are mutated in the human kappa light chain framework sequences back to the mouse sequences and are called back mutations. In some embodiments, the human kappa light chain framework sequences with mouse anti-CD122 CDR sequences grafted into it and back mutations incorporated into the framework sequence are used in a human light chain backbone to create a humanized light chain sequence. In some embodiments, the humanized kappa light chain sequence is used to create an anti-CD122 antibody. In some embodiments, LCDR sequences from one of series E1-E13 are grafted into a framework sequence to create a light chain variable domain sequence.

In some embodiments, the anti-CD122 antibody comprises a VH region and a VL region in which the sequence of the VH region comprises about 80%, 85%, 90%, 95%, 96% 97%, 98%, 99%, or 100% sequence identity to SEQ ID NOs: 68-80 and the sequence of the VL region comprises about 80%, 85%, 90%, 95%, 96% 97%, 98%, 99%, or 100% sequence identity to SEQ ID NOs: 81-93. In some embodiments, the VH region comprises a sequence selected from SEQ ID NOs: 68-80 (Table 4) and the VL region comprises a sequence selected from SEQ ID NOs: 81-93 (Table 5). VH sequences, names and correspondence SEQ ID NOs are listed in Table 4. VL sequences, names and correspondence SEQ ID NOs are listed in Table 5.

TABLE 4
VH Sequences

| SEQ ID | |
|---|---|
| NO: | VH Sequence |
| 68 | QVQLKESGPGLVAPSQSLSITCTVSGFSLTSYGIDWVRQPPGKGLEWLGIMWGGGS |
| | TNYNSALMSRLSISKDISKSQVFLKVNSLQTDDTAMYYCARRTYSDSYYYEMDYW |
| | GQGTSVTVSS |
| 69 | QVQLQQPGTELVKPGASVKMSCKASGYTFTSYWITWVKQRPGQGLEWIGDIYPGR |
| | GSTNYNEKFKSKATLTVDTSSSTAYMQLSSLTSEDSAVYYCARELGGFAYWGQGT |
| | LVTVSA |
| 70 | QVQLQQSGAELMKPGASVKISCKATGSTFNRYWIEWVKQRPGHGLEWIGEILPGS |
| | GNTNYNEKFKGKATFTADTSSNTAYMQLSSLTSEDSAVYYCARLDYYGSRYYFDY |
| | WGQGTTLTVSS |
| 71 | EVQLQQSGPELVKPGASVKMSCKASGFTFTDYNIHWVKQSHGKSLEWIGYINPNN |
| | GRSSYNLYFKDKATLTVNKSSSTAYMELRSLTSEDSAVYYCAREDWEGFYAMDY |
| | WGQGTSVTVSS |
| 72 | EVQLQQSGPELVKPGASMKISCKASGYSFTAYTMNWVRQSHGKNLEWIGLINPYN |
| | GYANYNQKFKGKATLTVDKSSSTAYMDLLSLTSEDSAVYYCARVGYYFDYWGQ |
| | GTTLTVSS |
| 73 | EVQLQQSGAELVRPGASVKLSCTSSGFNIKDDYMHWVKQRPEQGLEWIGWIDPEN |
| | GDTEYASKFQGKATITADTSSNTAYLQLNSLTSEDTAVYYCTGYFDYWGQGTTLT |
| | VSS |
| 74 | QVQLQQPGAELVKPGASVKMSCKASGYTFTSHWITWVKQRPGQGLEWIGDIYPGS |
| | GNTNYNEKFKSKATLTVDTSSSTAYMQLSSLTSEDSADYYCARERGGFDYWGQGT |
| | TLTVSS |
| 75 | DVILVESGGDLVKPGGSLKLSCAASGFTFSTFAMSWVRQTPEKRLEWVASITGDGG |
| | TYTYYSDSVKGRFTISRDNARNTLYLQMSSLRSEDTAFYYCARHSVSSWFAYWGQ |
| | GTLVTVSA |
| 76 | QVQLLQSDAELVKPGASVKISCKVSGFTFTDHTLHWMKQRPEQGLEWIGYIYPRD |
| | GYTKYNEKFKGKATLTADKSSSTAYMQLDSLTSEDSAVYFCARPTSLLRFPYWGQ |
| | GTLVTVSA |
| 77 | QVQLKESGPGLVAPSQSLSITCTVSGFSLTSYGVDWVRQPPGKGLEWLGVIWGGG |
| | GSTNYNSALMSRLSISKDNSKSQVFLKMNSLQTDDTAMYYCARHNYDGYYYSLD |
| | YWGQGTSVTVSS |
| 78 | QVQLKESGPGLVAPSQSLSITCTVSGFSLTSYGVDWVRQPPGKGLEWLGVIWGGG |
| | GSTNYNSALMSRLSISKDNSKSQVFLKMNSLQTDDTAMYYCARHNYDNYYYTLD |
| | YWGQGTSVTVSS |
| 79 | QVQLKESGPGLVAPSQSLSITCTVSGFSLTSYDISWIRQPPGKGLEWLGVIWTGGGT |
| | NYNSPFMSRLTISKDNSRSQVFLKMNSLQTDDTAMYYCVRDLFPYAMDYWGQGT |
| | SVTVSS |
| 80 | QVQLQQSGAELAKPGASVKMSCKASGYTFTAYWIHWVKQRPGQGLEWIGYIDPN |
| | SGYTDYNQIFKDKATLTADRSSSTAYMQLNSLTSEDSAVYYCARGHFGYDDSWG |
| | QGITLTVSS |

TABLE 5
VL Sequences

| SEQ ID | |
|---|---|
| NO: | VL Sequence |
| 81 | QIVLTQSPAIMSASPGEKVTMTCSASSSVSSRYLHWYQQKSGASPKLWIYGTSNLA |
| | SGVPARFSGSGSGTSYSLTISSVEAEDAATYYCQQYHSDPLTFGAGTKLELK |
| 82 | QIVLTQSPAIMSASLGERVTMTCTASSYVSSSYLHWYQQKPGSSPTLWIYTTSNLA |
| | SGVPARFSGSGSGTSYSLTISNMEAEDAATYYCHQYHLSPWTFGGGTKLEIK |
| 83 | DIQMTQTTSSLSASLGDRVTISCRASQDIRNYLNWYQQKPDGTLKLLIYYTSRLHS |
| | GVPSRFSGSGSGTDYSLTISNLEQEDVATYFCQQGDPLPPTFGGGTKVEIK |
| 84 | DIVMTQSQKFMSTSVGDRVSITCKASQNVDTDVSWYQQKPGKSPKTLIYWASNRF |
| | TGVPDRFTGSGSGTDFTLTITNVQSEDLADYFCEQYSSYPYTFGSGTKLEIK |
| 85 | ENVLTQSPTIMSASLGEKVTMSCRASSSVSYMHWYQQKSDASPKLWIYYTSNLAP |
| | GVPARFSASGSGNSYSLTISSMEGEDAATYYCQQFTSSPYTFGGGTKLEIK |
| 86 | DIQMNQSPSSLSASLGDTISISCRASQNINLWLNWYQQKPGNVPKLLIFKASNLHPG |
| | VPSRFSGSGSGTDFTLTISSLQPEDIATYYCLQGQSYPWTFGGGTKLEIK |
| 87 | DIQMTQTTSSLSASLGDRVTINCRASQDISNFLNWYQQKPDGTLKLLIYYTSGLHS |
| | GVPSRFSGSGSGTDYSLTISNLEEEDIATYFCQQDNNHPYTFGSGTKLEIK |
| 88 | ENVLTQSPAIMSASLGEKVTMSCRASSSVNYMYWYQQKSDASPKLWIYYTSNLAP |
| | GVPARFSGSGSGNSYSLTISSMEGEDAATYYCQQFTSSPWTFGGGTKLEIK |
| 89 | DIQMTQSPASLSASVGETVTITCRASEDIDSYLAWYQQKQGKSPQLLVYGATLLAD |
| | GVPSRFSGSGSGTQYSLKINSLQSEDVARYYCQHYYSIPYTFGSGTKLEIK |
| 90 | QIVLTQSPAIMSASPGEKVTMTCSASSSVSSRYLHWYQQKSGASPKLWIYGTSNLA |
| | SGVPARFSGSGSGTSYSLTISSVEAEDAATYYCQQYHGDPLTFGAGTKLELK |
| 91 | QIVLTQSPAIMSASPGEKVTMTCSASSSVSSRYLHWYQQKSGASPKLWIYGTSNLA |
| | SGVPARFSGSGSGTSYSLTISSVEAEDAASYYCQQYHGDPLTFGAGTKLELK |
| 92 | DILLTQSPAILSVSPGERVSLSCRASQTIGTSIHWYQQRTNGSIRLLIKYASESISGIPS |
| | RFSGSGFGTDFALSINSVESEDIADYFCQQTNSWPLTFGAGTKLELK |
| 93 | QIVLTQSPAIMSASPGEKVTITCSASSRVSYMHWFQQKPGTSPKLWIYSTSNLASGV |
| | PARFSGSGSGTSYSLTISRMEAEDAATYYCQQRSSYPLTFGAGTKLELK |

In some embodiments, the anti-CD122 antibody comprises a VH region that has HCDR sequences from a CDR series incorporated into a heavy chain framework sequence. In some embodiments, the anti-CD122 antibody comprises a VH region comprising HCDR sequences from series E1 incorporated into a framework sequence. In some embodiments, the anti-CD122 antibody comprises a VH region comprising HCDR sequences from series E2 incorporated into a framework sequence. In some embodiments, the anti-CD122 antibody comprises a VH region comprising HCDR sequences from series E3 incorporated into a framework sequence. In some embodiments, the anti-CD122 antibody comprises a VH region comprising HCDR sequences from series E4 incorporated into a framework sequence. In some embodiments, the anti-CD122 antibody comprises a VH region comprising HCDR sequences from series E5 incorporated into a framework sequence. In some embodiments, the anti-CD122 antibody comprises a VH region comprising HCDR sequences from series E6 incorporated into a framework sequence. In some embodiments, the anti-CD122 antibody comprises a VH region comprising HCDR sequences from series E7 incorporated into a framework sequence. In some embodiments, the anti-CD122 antibody comprises a VH region comprising HCDR sequences from series E8 incorporated into a framework sequence. In some embodiments, the anti-CD122 antibody comprises a VH region comprising HCDR sequences from series E9 incorporated into a framework sequence. In some embodiments, the anti-CD122 antibody comprises a VH region comprising HCDR sequences from series E10 incorporated into a framework sequence. In some embodiments, the anti-CD122 antibody comprises a VH region comprising HCDR sequences from series E11 incorporated into a framework sequence. In some embodiments, the anti-CD122 antibody comprises a VH region comprising HCDR sequences from series E12 incorporated into a framework sequence. In some embodiments, the anti-CD122 antibody comprises a VH region comprising HCDR sequences from series E13 incorporated into a framework sequence.

In some embodiments, the anti-CD122 antibody comprises a VL region that has LCDR sequences from a CDR series incorporated into a light chain framework sequence. In some embodiments, the anti-CD122 antibody comprises a VL region comprising HCDR sequences from series E1 incorporated into a framework sequence. In some embodiments, the anti-CD122 antibody comprises a VL region comprising HCDR sequences from series E2 incorporated into a framework sequence. In some embodiments, the anti-CD122 antibody comprises a VL region comprising HCDR sequences from series E3 incorporated into a framework sequence. In some embodiments, the anti-CD122 antibody comprises a VL region comprising HCDR sequences from series E4 incorporated into a framework sequence. In some embodiments, the anti-CD122 antibody comprises a VL region comprising HCDR sequences from series E5 incorporated into a framework sequence. In some embodiments, the anti-CD122 antibody comprises a VL region comprising HCDR sequences from series E6 incorporated into a framework sequence. In some embodiments, the anti-CD122 antibody comprises a VL region comprising HCDR sequences from series E7 incorporated into a framework sequence. In some embodiments, the anti-CD122 antibody comprises a VL region comprising HCDR sequences from series E8 incorporated into a framework sequence. In some embodiments, the anti-CD122 antibody comprises a VL region comprising HCDR sequences from series E9 incorporated into a framework sequence. In some embodiments, the anti-CD122 antibody comprises a VL region comprising HCDR sequences from series E10 incorporated into a framework sequence. In some embodiments, the anti-CD122 antibody comprises a VL region comprising HCDR sequences from series E11 incorporated into a framework sequence. In some embodiments, the anti-CD122 antibody comprises a VL region comprising HCDR sequences from series E12 incorporated into a framework sequence. In some embodiments, the anti-CD122 antibody comprises a VL region comprising HCDR sequences from series E13 incorporated into a framework sequence.

In some embodiments, the anti-CD122 antibody comprises a VH region comprising SEQ ID NO: 68 and a VL region comprising SEQ ID NO: 81. In some embodiments, the anti-CD122 antibody comprises a VH region comprising SEQ ID NO: 69 and a VL region comprising SEQ ID NO: 82. In some embodiments, the anti-CD122 antibody comprises a VH region comprising SEQ ID NO: 70 and a VL region comprising SEQ ID NO: 83. In some embodiments, the anti-CD122 antibody comprises a VH region comprising SEQ ID NO: 71 and a VL region comprising SEQ ID NO: 84. In some embodiments, the anti-CD122 antibody comprises a VH region comprising SEQ ID NO: 72 and a VL region comprising SEQ ID NO: 85. In some embodiments, the anti-CD122 antibody comprises a VH region comprising SEQ ID NO: 73 and a VL region comprising SEQ ID NO: 86. In some embodiments, the anti-CD122 antibody comprises a VH region comprising SEQ ID NO: 74 and a VL region comprising SEQ ID NO: 87. In some embodiments, the anti-CD122 antibody comprises a VH region comprising SEQ ID NO: 75 and a VL region comprising SEQ ID NO: 88. In some embodiments, the anti-CD122 antibody comprises a VH region comprising SEQ ID NO: 76 and a VL region comprising SEQ ID NO: 89. In some embodiments, the anti-CD122 antibody comprises a VH region comprising SEQ ID NO: 77 and a VL region comprising SEQ ID NO: 90. In some embodiments, the anti-CD122 antibody comprises a VH region comprising SEQ ID NO: 78 and a VL region comprising SEQ ID NO: 91. In some embodiments, the anti-CD122 antibody comprises a VH region comprising SEQ ID NO: 79 and a VL region comprising SEQ ID NO: 92. In some embodiments, the anti-CD122 antibody comprises a VH region comprising SEQ ID NO: 80 and a VL region comprising SEQ ID NO: 93.

In some embodiments, an anti-CD122 antibody described herein is a full-length antibody. In other aspects, the anti-CD122 antibody is an antigen-binding fragment thereof. In some cases, the anti-CD122 antibody is a humanized antibody or an antigen-binding fragment thereof, a chimeric antibody or an antigen-binding fragment thereof, a monoclonal antibody or an antigen-binding fragment thereof. In some cases, the anti-CD122 antibody is monovalent Fab′, F(ab)′3 fragments, single-chain variable fragment (scFv), (scFv)2, minibody, disulfide stabilized Fv protein (“dsFv”), single-domain antibody (sdAb), Ig NAR, camelid antibody or an antigen-binding fragment thereof, or a chemically-modified derivative thereof. In some embodiments, the antibody comprises an Immunoglobulin G (IgG) antibody or variant thereof. In some embodiments, the IgG antibody or variant thereof comprises an IgG1 antibody or variant thereof. In some embodiments, the IgG antibody or variant thereof comprises an IgG2 antibody or variant thereof. In some embodiments, the IgG antibody or variant thereof comprises an IgG3 antibody or variant thereof. In some embodiments, the IgG antibody or variant thereof comprises an IgG4 antibody or variant thereof. In some embodiments, the antibody comprises an IgA antibody. In some embodiments, the antibody comprises an IgM antibody. In some embodiments, the antibody comprises an IgE antibody.

In some cases, an anti-CD122 antibody comprises one or more mutations in a framework region, e.g., in the CH1 domain, CH2 domain, CH3 domain, hinge region, or a combination thereof. In some instances, the one or more mutations are to stabilize the antibody. In some instances, the one or more mutations are to increase half-life. In some instances, the one or more mutations are to modulate Fc receptor interactions. In some instances, the one or more mutations are to reduce or eliminate Fc effector functions such as FcγR, antibody-dependent cell-mediated cytotoxicity (ADCC), or complement-dependent cytotoxicity (CDC). In additional instances, the one or more mutations are to modulate glycosylation.

The term Fc region is used herein to specify a C-terminal region of an immunoglobulin heavy chain. The Fc region of the antibodies described herein can be a wild-type sequence. In some embodiments, the Fc region is human Fc region. In some embodiments, the Fc region is a mouse Fc region. Although the boundaries of the Fc region of an immunoglobulin heavy chain might vary, the human IgG heavy chain Fc region is typically defined to stretch from an amino acid residue at position Cys226, or from Pro230, to the carboxyl-terminus thereof. The Fc region of an immunoglobulin generally comprises two constant domains, CH2 and CH3. An Fc region can be present in a monomeric form or a dimeric form. The Fc region can bind (o various cell receptors, such as Fc receptors, and other immune molecules, including complement proteins. In some embodiments, the one or more mutations are located in the Fc region.

In some embodiments, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Urban et al., 2021 Front Immunol. November 25; 12:724361; Zhou et al., 2020 MAbs. January-December; 12(1):1814583; Reviewed in Kaneko and Niwa, 2011 Biodrugs, 25(1): 1-11.

In some embodiments, an anti-CD122 antibody described herein is a full-length antibody, comprising a heavy chain (HC) and a light chain (LC). In some embodiments, the HC comprises about 80%, 85%, 90%, 95%, 96% 97%, 98%, 99%, or 100% sequence identity to SEQ ID NOs: 94-101. In some embodiments, the HC comprises at least 80%, at least 85%, at least 90%, or at least 95% sequence identity sequence identity to SEQ ID NOs: 94-101. In some embodiments, the LC comprises about 80%, 85%, 90%, 95%, 96% 97%, 98%, 99%, or 100% sequence identity to SEQ ID NOs: 102-105. In some embodiments, the LC comprises at least 80%, at least 85%, at least 90%, or at least 95% sequence identity sequence identity to SEQ ID NOs: 102-105. In some cases, the heavy chain (HC) comprises a sequence selected from Table 6. In some cases, the light chain (LC) comprises a sequence selected from Table 7. In some embodiments, a HC with a VH domain with a mouse HC variable framework sequence and HCDR sequences is within an IgG1 backbone and is listed as SEQ ID NO: 94. In some embodiments, a HC with a VH domain with a mouse HC variable framework sequence and HCDR sequences is within an IgG4 backbone and is listed as SEQ ID NO: 98. In some embodiments, a LC with a VL domain with a mouse light chain variable framework sequence and LCDR sequences is within an kappa light chain backbone and is listed as SEQ ID NO: 102.

TABLE 6
anti-CD122 antibody heavy chain sequence

| SEQ ID | |
|---|---|
| NO: | Heavy Chain sequence |
| 94 | (X)ₙ₌₁₄₋₄₀ |
| | QVQLQQPGAELVKPGASVKMSCKASGYTFTSHWITWVKQRPGQGLEWIGDIYPGS |
| | GNTNYNEKFKSKATLTVDTSSSTAYMQLSSLTSEDSADYYCARERGGFDYWGQGT |
| | TLTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISK |
| | AKGQPREPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLSPG* |
| 95 | (X)ₙ₌₁₄₋₄₀ |
| | QVQLVQSGAEVKKPGASVKVSCKASGYTFTSHWISWVRQAPGQGLEWMGDIYPG |
| | SGNTNYNEKLQGRVTMTVDTSTSTAYMELRSLRSDDTAVYYCARERGGFDYWGQ |
| | GTLVTVSSASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKVEPKSCDKT |
| | HTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSREEMTKNQVSLTCLVKGFYPSDIAVEWESNGQPENNY |
| | KTTPPVLDSDGSFFLYSKLTVDKSRWQQGNVFSCSVMHEALHNHYTQKSLSLSPG* |
| 96 | (X)ₙ₌₁₄₋₄₀ |
| | QVQLVQSGAEVKKPGASVKVSCKASGYTFTSHWITWVRQAPGQGLEWMGDIYPG |
| | SGNTNYNEKFQGRVTLTVDTSISTAYMELSRLRSDDTVVYYCARERGGFDYWGQG |
| | TLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTIS |
| | KAKGQPREPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLSLSPG* |
| 97 | (X)ₙ₌₁₄₋₄₀ |
| | QVQLVQSGAEVKKPGASVKVSCKASGYTFTSHWITWVRQAPGQRLEWMGDIYPG |
| | SGNTNYNEKFQGRVTITVDTSASTAYMELSSLRSEDTAVYYCARERGGFDYWGQG |
| | TLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTH |
| | TCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTIS |
| | KAKGQPREPQVYTLPPSREEMTKNQVSLTCLVKGFYPSDIAVEWESNGQPENNYKT |
| | TPPVLDSDGSFFLYSKLTVDKSRWQQGNVFSCSVMHEALHNHYTQKSLSLSPG* |
| 98 | (X)ₙ₌₁₄₋₄₀ |
| | QVQLQQPGAELVKPGASVKMSCKASGYTFTSHWITWVKQRPGQGLEWIGDIYPGS |
| | GNTNYNEKFKSKATLTVDTSSSTAYMQLSSLTSEDSADYYCARERGGFDYWGQGT |
| | TLTVSSASTKGPSVFPLAPCSRSTSESTAALGCLVKDYFPEPVTVSWNSGALTSGVH |
| | TFPAVLQSSGLYSLSSVVTVPSSSLGTKTYTCNVDHKPSNTKVDKRVESKYGPPCPP |
| | CPAPEFLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEKTISKAK |
| | GQPREPQVYTLPPSQEEMTKNQVSLTCLVKGFYPSDIAVEWESNGQPENNYKTTPP |
| | VLDSDGSFFLYSRLTVDKSRWQEGNVFSCSVMHEALHNHYTQKSLSLSLG* |
| 99 | (X)ₙ₌₁₄₋₄₀ |
| | QVQLVQSGAEVKKPGASVKVSCKASGYTFTSHWISWVRQAPGQGLEWMGDIYPG |
| | SGNTNYNEKLQGRVTMTVDTSTSTAYMELRSLRSDDTAVYYCARERGGFDYWGQ |
| | GTLVTVSSASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVESKYGPPC |
| | PPCPAPEFLGGPSVFLFPPKPKDTLMISRTPEVTCVVVDVSQEDPEVQFNWYVDGVE |
| | VHNAKTKPREEQFNSTYRVVSVLTVLHQDWLNGKEYKCKVSNKGLPSSIEKTISKA |
| | KGQPREPQVYTLPPSQEEMTKNQVSLTCLVKGFYPSDIAVEWESNGQPENNYKTTP |
| | PVLDSDGSFFLYSRLTVDKSRWQEGNVFSCSVMHEALHNHYTQKSLSLSLG* |
| 100 | (X)ₙ₌₁₄₋₄₀ |
| | QVQLVQSGAEVKKPGASVKVSCKASGYTFTSHWITWVRQAPGQGLEWMGDIYPG |
| | SGNTNYNEKFQGRVTLTVDTSISTAYMELSRLRSDDTVVYYCARERGGFDYWGQG |
| | TLVTVSSASTKGPSVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEKTISKAK |
| | GQPREPQVYTLPPSQEEMTKNQVSLTCLVKGFYPSDIAVEWESNGQPENNYKTTPP |
| | VLDSDGSFFLYSRLTVDKSRWQEGNVFSCSVMHEALHNHYTQKSLSLSLG* |
| 101 | (X)ₙ₌₁₄₋₄₀ |
| | QVQLVQSGAEVKKPGASVKVSCKASGYTFTSHWITWVRQAPGQRLEWMGDIYPG |
| | SGNTNYNEKFQGRVTITVDTSASTAYMELSSLRSEDTAVYYCARERGGFDYWGQG |
| | TLVTVSSASTKGPSVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPSSIEKTISKAK |
| | GQPREPQVYTLPPSQEEMTKNQVSLTCLVKGFYPSDIAVEWESNGQPENNYKTTPP |
| | VLDSDGSFFLYSRLTVDKSRWQEGNVFSCSVMHEALHNHYTQKSLSLSLG* |

(X)ₙ₌₁₄₋₄₀ is a leader sequence

TABLE 7
anti-CD122 antibody light chain sequence

| SEQ ID | |
|---|---|
| NO: | Light Chain sequence |
| 102 | (X)ₙ₌₁₄₋₄₀ |
| | DIQMTQTTSSLSASLGDRVTINCRASQDISNFLNWYQQKPDGTLKLLIYYTSGLH |
| | SGVPSRFSGSGSGTDYSLTISNLEEEDIATYFCQQDNNHPYTFGSGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 103 | (X)ₙ₌₁₄₋₄₀ |
| | DIQMTQSPSSLSASVGDRVTITCRASQDISNFLNWYQQKPEGTLKLLIYYTSGLH |
| | SGVPSRFSGSGSGTDFTLTISSLQPEDVATYYCQQDNNHPYTFGQGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 104 | (X)ₙ₌₁₄₋₄₀ |
| | DIQMTQSPSSLSASVGDRVTITCQASQDISNFLNWYQQKPGKTLKLLIYYTSGLH |
| | TGVPSRFSGSGSGTDFTFTISSLQPEDIATYYCQQDNNHPYTFGQGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 105 | (X)ₙ₌₁₄₋₄₀ |
| | DIQMTQSPSSLSASVGDRVTITCRASQDISNFLNWYQQKPGKAPKLLIYYTSGLH |
| | SGVPSRFSGSGSGTDFTLTISSLQPEDFATYYCQQDNNHPYTFGQGTKLEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |

(X)ₙ₌₁₄₋₄₀ is a leader sequence

In some embodiments, an anti-CD122 antibody described herein is a full-length antibody, comprising a heavy chain (HC) and a light chain (LC). In some embodiments, the full-length antibody comprises the HC comprising SEQ ID NO: 94 and the LC comprising SEQ ID NO: 102. In some embodiments, the full-length antibody comprises the HC comprising SEQ ID NO: 95 and the LC comprising SEQ ID NO: 103. In some embodiments, the full-length antibody comprises the HC comprising SEQ ID NO: 95 and the LC comprising SEQ ID NO: 104. In some embodiments, the full-length antibody comprises the HC comprising SEQ ID NO: 95 and the LC comprising SEQ ID NO: 105. In some embodiments, the full-length antibody comprises the HC comprising SEQ ID NO: 96 and the LC comprising SEQ ID NO: 103. In some embodiments, the full-length antibody comprises the HC comprising SEQ ID NO: 96 and the LC comprising SEQ ID NO: 104. In some embodiments, the full-length antibody comprises the HC comprising SEQ ID NO: 96 and the LC comprising SEQ ID NO: 105. In some embodiments, the full-length antibody comprises the HC comprising SEQ ID NO: 97 and the LC comprising SEQ ID NO: 103. In some embodiments, the full-length antibody comprises the HC comprising SEQ ID NO: 97 and the LC comprising SEQ ID NO: 104. In some embodiments, the full-length antibody comprises the HC comprising SEQ ID NO: 97 and the LC comprising SEQ ID NO: 105. In some embodiments, the full-length antibody comprises the HC comprising SEQ ID NO: 98 and the LC comprising SEQ ID NO: 102. In some embodiments, the full-length antibody comprises the HC comprising SEQ ID NO: 99 and the LC comprising SEQ ID NO: 103. In some embodiments, the full-length antibody comprises the HC comprising SEQ ID NO: 99 and the LC comprising SEQ ID NO: 104. In some embodiments, the full-length antibody comprises the HC comprising SEQ ID NO: 99 and the LC comprising SEQ ID NO: 105. In some embodiments, the full-length antibody comprises the HC comprising SEQ ID NO: 100 and the LC comprising SEQ ID NO: 103. In some embodiments, the full-length antibody comprises the HC comprising SEQ ID NO: 100 and the LC comprising SEQ ID NO: 104. In some embodiments, the full-length antibody comprises the HC comprising SEQ ID NO: 100 and the LC comprising SEQ ID NO: 105. In some embodiments, the full-length antibody comprises the HC comprising SEQ ID NO: 101 and the LC comprising SEQ ID NO: 103. In some embodiments, the full-length antibody comprises the HC comprising SEQ ID NO: 101 and the LC comprising SEQ ID NO: 104. In some embodiments, the full-length antibody comprises the HC comprising SEQ ID NO: 101 and the LC comprising SEQ ID NO: 105.

In some embodiments, the anti-CD122 antibody heavy chain and the anti-CD122 antibody light chain have a leader sequence near an amino-terminal region of each polypeptide. In some embodiments, the leader sequence is N-terminal to an anti-CD122 antibody heavy chain sequence. In some embodiments, the leader sequence is N-terminal to an anti-CD122 antibody heavy chain sequence provided in Table 6. In some embodiments, the leader sequence is N-terminal to an anti-CD122 antibody light chain sequence. In some embodiments, the leader sequence is N-terminal to an anti-CD122 antibody light chain sequence provided in Table 7. In some embodiments, (X)ₙ₌₁₄₋₄₀ is used to represent amino acids of a leader sequence at the amino-terminal end of an anti-CD122 antibody heavy chain sequence. In some embodiments, (X)ₙ₌₁₄₋₄₀ is used to represent amino acids of a leader sequence at the amino-terminal end of an anti-CD122 antibody light chain sequence. In some embodiments, the leader sequence is a length of between 14-40 amino acids. In some embodiments, the leader sequence is a length of between 15-35 amino acids. In some embodiments, the leader sequence of an anti-CD122 antibody heavy chain and an anti-CD122 antibody light chain paired to form an anti-CD122 antibody are identical to each other. In some embodiments, the leader sequence of an anti-CD122 antibody heavy chain and an anti-CD122 antibody light chain paired to form an anti-CD122 antibody are not identical to each other. In some embodiments, the leader sequence comprises an amino acid sequence listed in Table 8. In some embodiments, the anti-CD122 antibody heavy chain contains an additional amino acid sequence N-terminal to the leader sequence. In some embodiments, the anti-CD122 antibody heavy chain contains an additional amino acid sequence immediately after the leader sequence. In some embodiments, the additional amino acid sequence immediately after the leader sequence is before a start of a heavy chain variable domain. In some embodiments, the anti-CD122 antibody light chain contains an additional amino acid sequence N-terminal to the leader sequence. In some embodiments, the anti-CD122 antibody light chain contains an additional amino acid sequence immediately after the leader sequence. In some embodiments, the additional amino acid sequence immediately after the leader sequence is before a start of a light chain variable domain. In some embodiments, the leader sequence used N-terminal to the anti-CD122 antibody heavy chain sequence comprises a leader sequence comprising SEQ ID NO: 106. In some embodiments, the leader sequence used N-terminal to the anti-CD122 antibody heavy chain sequence comprises a leader sequence comprising SEQ ID NO: 107. In some embodiments, the leader sequence used N-terminal to the anti-CD122 antibody heavy chain sequence comprises a leader sequence comprising SEQ ID NO: 108. In some embodiments, the leader sequence used N-terminal to the anti-CD122 antibody heavy chain sequence comprises a leader sequence comprising SEQ ID NO: 109.

In some embodiments, the leader sequence used N-terminal to the anti-CD122 antibody light chain sequence comprises a leader sequence comprising SEQ ID NO:106. In some embodiments, the leader sequence used N-terminal to the anti-CD122 antibody light chain sequence comprises a leader sequence comprising SEQ ID NO: 107. In some embodiments, the leader sequence used N-terminal to the anti-CD122 antibody light chain sequence comprises a leader sequence comprising SEQ ID NO: 108. In some embodiments, the leader sequence used N-terminal to the anti-CD122 antibody light chain sequence comprises a leader sequence comprising SEQ ID NO:109.

TABLE 8
Leader Sequences for use with heavy chains
and light chains

| SEQ ID NO: | Leader Sequence |
|---|---|
| 106 | MDPKGSLSWRILLFLSLAFELSYG |
| 107 | METDTLLLWVLLLWVPGSTG |
| 108 | MGWSLILLFLVAVATRVHS |
| 109 | MRVPAQLLGLLLLWLPGARC |

In some embodiments, the anti-CD122 antibody comprises i) a heavy chain comprising a variable heavy chain (VH) domain and ii) a light chain comprising a variable light chain (VL) domain. In some embodiments, VH domain comprises heavy chain CDR1 (HCDR1) sequence comprising a sequence selected from SEQ ID NOs: 110-115, heavy chain CDR2 (HCDR2) sequence comprising a sequence selected from SEQ ID NOs: 116-123, and heavy chain CDR3 (HCDR3) sequence comprising a sequence selected from SEQ ID NOs: 124-130. In some embodiments, VL domain comprises light chain CDR1 (LCDR1) sequence comprising a sequence selected from SEQ ID NOs: 131-140, light chain CDR2 (LCDR2) sequence comprising a sequence selected from SEQ ID NOs: 141-146, and light chain CDR3 (LCDR3) sequence comprising a sequence selected from SEQ ID NOs: 147-151.

In some embodiments, the VH region of the anti-CD122 antibody comprises HCDR1, HCDR2, and HCDR3 sequences selected from Table 9.

TABLE 9
HCDR Sequences

| SEQ ID NO: | HCDR1 Sequence |
|---|---|
| 110 | SYGVH |
| 111 | SYAVH |
| 112 | SYAMH |
| 113 | SYGMH |
| 114 | DFYME |
| 115 | NDNHWWN |
| SEQ ID NO: | HCDR2 Sequence |
| 116 | VIWSGGSTDYNAAFIS |
| 117 | AIWSGGSTDYNAAVKG |
| 118 | AIWSGGSTDYAASVKG |
| 119 | AIWSGGSTDYADAVKG |
| 120 | VIWSGGSTDYADSVKG |
| 121 | VIWSGGSTDYADAVKG |
| 122 | ASRNKANDYTTEYSASVKG |
| 123 | YIDSSGSSDNNPSLKS |
| SEQ ID NO: | HCDR3 Sequence |
| 124 | AGDYNYDGFAY |
| 125 | AGDANYDGFAY |
| 126 | AGDYEYDGFAY |
| 127 | AGDKNYDGFAY |
| 128 | AGDQNYDGFAY |
| 129 | SYYRYDGMDY |
| 130 | GGGRDYYGMDY |

In some embodiments, the VH region comprises HCDR1 sequence comprising SEQ ID NO: 110; HCDR2 sequence comprising SEQ ID NO: 116; and HCDR3 sequence comprising SEQ ID NO: 124. In some embodiments, the VH region comprises HCDR1 sequence comprising SEQ ID NO: 111; HCDR2 sequence comprising SEQ ID NO: 116; and HCDR3 sequence comprising SEQ ID NO: 125. In some embodiments, the VH region comprises HCDR1 sequence comprising SEQ ID NO: 111; HCDR2 sequence comprising SEQ ID NO: 117; and HCDR3 sequence comprising SEQ ID NO: 125. In some embodiments, the VH region comprises HCDR1 sequence comprising SEQ ID NO: 110; HCDR2 sequence comprising SEQ ID NO: 118; and HCDR3 sequence comprising SEQ ID NO: 124. In some embodiments, the VH region comprises HCDR1 sequence comprising SEQ ID NO: 112; HCDR2 sequence comprising SEQ ID NO: 119; and HCDR3 sequence comprising SEQ ID NO: 126. In some embodiments, the VH region comprises HCDR1 sequence comprising SEQ ID NO: 113; HCDR2 sequence comprising SEQ ID NO: 120; and HCDR3 sequence comprising SEQ ID NO: 124. In some embodiments, the VH region comprises HCDR1 sequence comprising SEQ ID NO: 110; HCDR2 sequence comprising SEQ ID NO: 119; and HCDR3 sequence comprising SEQ ID NO: 127. In some embodiments, the VH region comprises HCDR1 sequence comprising SEQ ID NO: 110; HCDR2 sequence comprising SEQ ID NO: 121; and HCDR3 sequence comprising SEQ ID NO: 128. In some embodiments, the VH region comprises HCDR1 sequence comprising SEQ ID NO: 114; HCDR2 sequence comprising SEQ ID NO: 122; and HCDR3 sequence comprising SEQ ID NO: 129. In some embodiments, the VH region comprises HCDR1 sequence comprising SEQ ID NO: 115; HCDR2 sequence comprising SEQ ID NO: 122; and HCDR3 sequence comprising SEQ ID NO: 129.

In some embodiments, the VL region of the anti-CD122 antibody comprises LCDR1, LCDR2, and LCDR3 sequences selected from Table 10.

TABLE 10
LCDR Sequences

| SEQ ID NO: | LCDR1 Sequence |
|---|---|
| 131 | SGSSSVSFMY |
| 132 | QASQSVSFLY |
| 133 | QASSSVSFMY |
| 134 | QASQDISFMY |
| 135 | QASQSVSHLY |
| 136 | QASQSVSYMY |
| 137 | QASSSISYMY |
| 138 | QASSSVSHLY |
| 139 | SAISSVSYMY |
| 140 | RASQSVSTSSYSYVH |
| SEQ ID NO: | LCDR2 Sequence |
| 141 | DTSNLAS |
| 142 | DTSNLAT |
| 143 | DASNLAT |
| 144 | DTSNLET |
| 145 | DTSNLVS |
| 146 | YASNLES |
| SEQ ID NO: | LCDR3 Sequence |
| 147 | QQWSTYPLT |
| 148 | QQWDTYPLT |
| 149 | QQWDNYPLT |
| 150 | QQWNTYPYT |
| 151 | QHSWDIPFT |

In some embodiments, the VL region comprises LCDR1 sequence comprising SEQ ID NO: 131; LCDR2 sequence comprising SEQ ID NO: 141; and LCDR3 sequence comprising SEQ ID NO: 147. In some embodiments, the VL region comprises LCDR1 sequence comprising SEQ ID NO: 132; LCDR2 sequence comprising SEQ ID NO: 141; and LCDR3 sequence comprising SEQ ID NO: 147. In some embodiments, the VL region comprises LCDR1 sequence comprising SEQ ID NO: 133; LCDR2 sequence comprising SEQ ID NO: 141; and LCDR3 sequence comprising SEQ ID NO: 147. In some embodiments, the VL region comprises LCDR1 sequence comprising SEQ ID NO: 132; LCDR2 sequence comprising SEQ ID NO: 142; and LCDR3 sequence comprising SEQ ID NO: 148. In some embodiments, the VL region comprises LCDR1 sequence comprising SEQ ID NO: 134; LCDR2 sequence comprising SEQ ID NO: 143; and LCDR3 sequence comprising SEQ ID NO: 149. In some embodiments, the VL region comprises LCDR1 sequence comprising SEQ ID NO: 135; LCDR2 sequence comprising SEQ ID NO: 142; and LCDR3 sequence comprising SEQ ID NO: 147. In some embodiments, the VL region comprises LCDR1 sequence comprising SEQ ID NO: 136; LCDR2 sequence comprising SEQ ID NO: 143; and LCDR3 sequence comprising SEQ ID NO: 148. In some embodiments, the VL region comprises LCDR1 sequence comprising SEQ ID NO: 137; LCDR2 sequence comprising SEQ ID NO: 143; and LCDR3 sequence comprising SEQ ID NO: 149. In some embodiments, the VL region comprises LCDR1 sequence comprising SEQ ID NO: 138; LCDR2 sequence comprising SEQ ID NO: 144; and LCDR3 sequence comprising SEQ ID NO: 148. In some embodiments, the VL region comprises LCDR1 sequence comprising SEQ ID NO: 139; LCDR2 sequence comprising SEQ ID NO: 145; and LCDR3 sequence comprising SEQ ID NO: 150. In some embodiments, the VL region comprises LCDR1 sequence comprising SEQ ID NO: 140; LCDR2 sequence comprising SEQ ID NO: 146; and LCDR3 sequence comprising SEQ ID NO: 151.

In some embodiments, the anti-CD122 antibody comprises a VH region and a VL region, in which the VH region comprises HCDR1 sequence comprising SEQ ID NO: 110; HCDR2 sequence comprising SEQ ID NO: 116; and HCDR3 sequence comprising SEQ ID NO: 124 and in which the VL region comprises LCDR1 sequence comprising SEQ ID NO: 131; LCDR2 sequence comprising SEQ ID NO: 141; and LCDR3 sequence comprising SEQ ID NO: 147. In some embodiments, the anti-CD122 antibody comprises a VH region and a VL region, in which the VH region comprises HCDR1 sequence comprising SEQ ID NO: 111; HCDR2 sequence comprising SEQ ID NO: 116; and HCDR3 sequence comprising SEQ ID NO: 125 and in which the VL region comprises LCDR1 sequence comprising SEQ ID NO: 132; LCDR2 sequence comprising SEQ ID NO: 141; and LCDR3 sequence comprising SEQ ID NO: 147. In some embodiments, the anti-CD122 antibody comprises a VH region and a VL region, in which the VH region comprises HCDR1 sequence comprising SEQ ID NO: 111; HCDR2 sequence comprising SEQ ID NO: 116; and HCDR3 sequence comprising SEQ ID NO: 125 and in which the VL region comprises LCDR1 sequence comprising SEQ ID NO: 133; LCDR2 sequence comprising SEQ ID NO: 141; and LCDR3 sequence comprising SEQ ID NO: 147. In some embodiments, the anti-CD122 antibody comprises a VH region and a VL region, in which the VH region comprises HCDR1 sequence comprising SEQ ID NO: 111; HCDR2 sequence comprising SEQ ID NO: 117; and HCDR3 sequence comprising SEQ ID NO: 125 and in which the VL region comprises LCDR1 sequence comprising SEQ ID NO: 132; LCDR2 sequence comprising SEQ ID NO: 142; and LCDR3 sequence comprising SEQ ID NO: 148. In some embodiments, the anti-CD122 antibody comprises a VH region and a VL region, in which the VH region comprises HCDR1 sequence comprising SEQ ID NO: 112; HCDR2 sequence comprising SEQ ID NO: 119; and HCDR3 sequence comprising SEQ ID NO: 126 and in which the VL region comprises LCDR1 sequence comprising SEQ ID NO: 135; LCDR2 sequence comprising SEQ ID NO: 142; and LCDR3 sequence comprising SEQ ID NO: 147. In some embodiments, the anti-CD122 antibody comprises a VH region and a VL region, in which the VH region comprises HCDR1 sequence comprising SEQ ID NO: 113; HCDR2 sequence comprising SEQ ID NO: 120; and HCDR3 sequence comprising SEQ ID NO: 124 and in which the VL region comprises LCDR1 sequence comprising SEQ ID NO: 136; LCDR2 sequence comprising SEQ ID NO: 143; and LCDR3 sequence comprising SEQ ID NO: 148. In some embodiments, the anti-CD122 antibody comprises a VH region and a VL region, in which the VH region comprises HCDR1 sequence comprising SEQ ID NO: 110; HCDR2 sequence comprising SEQ ID NO: 119; and HCDR3 sequence comprising SEQ ID NO: 127 and in which the VL region comprises LCDR1 sequence comprising SEQ ID NO: 137; LCDR2 sequence comprising SEQ ID NO: 143; and LCDR3 sequence comprising SEQ ID NO: 149. In some embodiments, the anti-CD122 antibody comprises a VH region and a VL region, in which the VH region comprises HCDR1 sequence comprising SEQ ID NO: 110; HCDR2 sequence comprising SEQ ID NO: 121; and HCDR3 sequence comprising SEQ ID NO: 128 and in which the VL region comprises LCDR1 sequence comprising SEQ ID NO: 138; LCDR2 sequence comprising SEQ ID NO: 144; and LCDR3 sequence comprising SEQ ID NO: 148. In some embodiments, the anti-CD122 antibody comprises a VH region and a VL region, in which the VH region comprises HCDR1 sequence comprising SEQ ID NO: 114; HCDR2 sequence comprising SEQ ID NO: 122; and HCDR3 sequence comprising SEQ ID NO: 129 and in which the VL region comprises LCDR1 sequence comprising SEQ ID NO: 139; LCDR2 sequence comprising SEQ ID NO: 145; and LCDR3 sequence comprising SEQ ID NO: 150. In some embodiments, the anti-CD122 antibody comprises a VH region and a VL region, in which the VH region comprises HCDR1 sequence comprising SEQ ID NO: 115; HCDR2 sequence comprising SEQ ID NO: 123; and HCDR3 sequence comprising SEQ ID NO: 130 and in which the VL region comprises LCDR1 sequence comprising SEQ ID NO: 140; LCDR2 sequence comprising SEQ ID NO: 146; and LCDR3 sequence comprising SEQ ID NO: 151.

In some embodiments, the anti-CD122 antibody comprises a series of CDR sequences. In some embodiments, the series of CDR sequences comprises an HCDR1 sequence, an HCDR2 sequence, an HCDR3 sequence, an LCDR1 sequence, an LCDR2 sequence, and an LCDR3 sequence. The series of CDR sequences for each anti-CD122 antibody described herein is listed in Table 11A. The names for the series of CDR sequences are E14-E40.

TABLE 11A
Series of CDR sequences for anti-CD122 antibodies

| Name | HCDR1 | HCDR2 | HCDR3 | LCDR1 | LCDR2 | LCDR3 |
|---|---|---|---|---|---|---|
| E14 | SEQ ID | SEQ ID | SEQ ID | SEQ ID | SEQ ID | SEQ ID |
| | NO: 110 | NO: 116 | NO: 124 | NO: 131 | NO: 141 | NO: 147 |
| E15 | SEQ ID | SEQ ID | SEQ ID | SEQ ID | SEQ ID | SEQ ID |
| | NO: 111 | NO: 116 | NO: 125 | NO: 132 | NO: 141 | NO: 147 |
| E16 | SEQ ID | SEQ ID | SEQ ID | SEQ ID | SEQ ID | SEQ ID |
| | NO: 111 | NO: 116 | NO: 125 | NO: 133 | NO: 141 | NO: 147 |
| E17 | SEQ ID | SEQ ID | SEQ ID | SEQ ID | SEQ ID | SEQ ID |
| | NO: 111 | NO: 117 | NO: 125 | NO: 132 | NO: 142 | NO: 148 |
| E18 | SEQ ID | SEQ ID | SEQ ID | SEQ ID | SEQ ID | SEQ ID |
| | NO: 110 | NO: 118 | NO: 124 | NO: 134 | NO: 143 | NO: 149 |
| E19 | SEQ ID | SEQ ID | SEQ ID | SEQ ID | SEQ ID | SEQ ID |
| | NO: 112 | NO: 119 | NO: 126 | NO: 135 | NO: 142 | NO: 147 |
| E20 | SEQ ID | SEQ ID | SEQ ID | SEQ ID | SEQ ID | SEQ ID |
| | NO: 113 | NO: 120 | NO: 124 | NO: 136 | NO: 143 | NO: 148 |
| E21 | SEQ ID | SEQ ID | SEQ ID | SEQ ID | SEQ ID | SEQ ID |
| | NO: 110 | NO: 119 | NO: 127 | NO: 137 | NO: 143 | NO: 149 |
| E22 | SEQ ID | SEQ ID | SEQ ID | SEQ ID | SEQ ID | SEQ ID |
| | NO: 110 | NO: 121 | NO: 128 | NO: 138 | NO: 144 | NO: 148 |
| E23 | SEQ ID | SEQ ID | SEQ ID | SEQ ID | SEQ ID | SEQ ID |
| | NO: 114 | NO: 122 | NO: 129 | NO: 139 | NO: 145 | NO: 150 |
| E24 | SEQ ID | SEQ ID | SEQ ID | SEQ ID | SEQ ID | SEQ ID |
| | NO: 115 | NO: 123 | NO: 130 | NO: 140 | NO: 146 | NO: 151 |
| E25 | SEQ ID | SEQ ID | SEQ ID | SEQ ID | SEQ ID | SEQ ID |
| | NO: 110 | NO: 116 | NO: 125 | NO: 132 | NO: 141 | NO: 147 |
| E26 | SEQ ID | SEQ ID | SEQ ID | SEQ ID | SEQ ID | SEQ ID |
| | NO: 110 | NO: 116 | NO: 125 | NO: 133 | NO: 141 | NO: 147 |
| E27 | SEQ ID | SEQ ID | SEQ ID | SEQ ID | SEQ ID | SEQ ID |
| | NO: 110 | NO: 116 | NO: 124 | NO: 132 | NO: 141 | NO: 147 |
| E28 | SEQ ID | SEQ ID | SEQ ID | SEQ ID | SEQ ID | SEQ ID |
| | NO: 110 | NO: 116 | NO: 124 | NO: 133 | NO: 141 | NO: 147 |
| E29 | SEQ ID | SEQ ID | SEQ ID | SEQ ID | DTS | SEQ ID |
| | NO: 202 | NO: 210 | NO: 218 | NO: 41 | | NO: 150 |
| E30 | SEQ ID | SEQ ID | SEQ ID | SEQ ID | SEQ ID | SEQ ID |
| | NO: 203 | NO: 211 | NO: 129 | NO: 139 | NO: 145 | NO: 150 |
| E31 | SEQ ID | SEQ ID | SEQ ID | SEQ ID | SEQ ID | SEQ ID |
| | NO: 204 | NO: 212 | NO: 129 | NO: 139 | NO: 145 | NO: 150 |
| E32 | SEQ ID | SEQ ID | SEQ ID | SEQ ID | SEQ ID | SEQ ID |
| | NO: 205 | NO: 213 | NO: 219 | NO: 222 | NO: 225 | NO: 227 |
| E33 | SEQ ID | SEQ ID | SEQ ID | SEQ ID | DTS | SEQ ID |
| | NO: 206 | NO: 214 | NO: 220 | NO: 223 | | NO: 147 |
| E34 | SEQ ID | SEQ ID | SEQ ID | SEQ ID | SEQ ID | SEQ ID |
| | NO: 207 | NO: 215 | NO: 125 | NO: 132 | NO: 141 | NO: 147 |
| E35 | SEQ ID | SEQ ID | SEQ ID | SEQ ID | SEQ ID | SEQ ID |
| | NO: 208 | NO: 216 | NO: 125 | NO: 132 | NO: 141 | NO: 147 |
| E36 | SEQ ID | SEQ ID | SEQ ID | SEQ ID | SEQ ID | SEQ ID |
| | NO: 209 | NO: 217 | NO: 221 | NO: 224 | NO: 228 | NO: 230 |
| E37 | SEQ ID | SEQ ID | SEQ ID | SEQ ID | DTS | SEQ ID |
| | NO: 206 | NO: 214 | NO: 220 | NO: 225 | | NO: 147 |
| E38 | SEQ ID | SEQ ID | SEQ ID | SEQ ID | SEQ ID | SEQ ID |
| | NO: 207 | NO: 215 | NO: 125 | NO: 133 | NO: 141 | NO: 147 |
| E39 | SEQ ID | SEQ ID | SEQ ID | SEQ ID | SEQ ID | SEQ ID |
| | NO: 208 | NO: 216 | NO: 125 | NO: 133 | NO: 141 | NO: 147 |
| E40 | SEQ ID | SEQ ID | SEQ ID | SEQ ID | SEQ ID | SEQ ID |
| | NO: 209 | NO: 217 | NO: 221 | NO: 226 | NO: 228 | NO: 230 |

In some embodiments, the anti-CD122 antibody CDR sequences can be defined by an antibody numbering scheme. In embodiments, the antibody numbering scheme is Kabat. In embodiments, the antibody numbering scheme is IMGT. In embodiments, the antibody numbering scheme is AbM. In embodiments, the antibody numbering scheme is Chothia. In embodiments, the antibody numbering scheme is Contact. In some embodiments, CDR sequences of anti-CD122 antibodies are numbering according to Table 11B.

TABLE 11B
CDR sequences for anti-CD122 antibody heavy chain variable domains
and light chain variable domains

| SEQ | | | | | | | |
|---|---|---|---|---|---|---|---|
| ID | | | | | | | |
| NO. of | | | | | | | |
| VH | | | | | | | |
| domain | | | | | | | |
| from | | SEQ | | SEQ | | SEQ | |
| Table | Numbering | ID | | ID | | ID | |
| 12 | Scheme | NO: | HCDR1 | NO: | HCDR2 | NO: | HCDR3 |
| 156 | Kabat | 114 | DFYME | 122 | ASRNKANDYTTEYSASV | 129 | SYYRYDGMDY |
| | | | | | KG | | |
| | IMGT | 202 | GFTFSDFY | 210 | SRNKANDYTT | 218 | ARSYYRYDGMD |
| | | | | | | | Y |
| | AbM | 203 | GFTFSDFYM | 211 | ASRNKANDYTTE | 129 | SYYRYDGMDY |
| | | | E | | | | |
| | Chothia | 204 | GFTFSDF | 212 | RNKANDYT | 129 | SYYRYDGMDY |
| | Contact | 205 | SDFYME | 213 | WIAASRNKANDYTTE | 219 | ARSYYRYDGMD |
| 153 | Kabat | 111 | SYAVH | 116 | VIWSGGSTDYNAAFIS | 125 | AGDANYDGFAY |
| | IMGT | 206 | GFTVTSYA | 214 | IWSGGST | 220 | ARAGDANYDGF |
| | | | | | | | AY |
| | AbM | 207 | GFTVTSYAV | 215 | VIWSGGSTD | 125 | AGDANYDGFAY |
| | | | H | | | | |
| | Chothia | 208 | GFTVTSY | 216 | WSGGS | 125 | AGDANYDGFAY |
| | Contact | 209 | TSYAVH | 217 | WLGVIWSGGSTD | 221 | ARAGDANYDGF |
| | | | | | | | A |
| SEQ | | | | | | | |
| ID | | | | | | | |
| NO. of | | | | | | | |
| VL | | | | | | | |
| domain | | | | | | | |
| from | | SEQ | | SEQ | | SEQ | |
| Table | Numbering | ID | | ID | | ID | |
| 13 | Scheme | NO: | HCDR1 | NO: | HCDR2 | NO: | HCDR3 |
| 163 | Kabat | 139 | SAISSVSYM | 145 | DTSNLVS | 150 | QQWNTYPYT |
| | | | Y | | | | |
| | IMGT | 41 | SSVSY | — | DTS | 150 | QQWNTYPYT |
| | AbM | 139 | SAISSVSYM | 145 | DTSNLVS | 150 | QQWNTYPYT |
| | | | Y | | | | |
| | Chothia | 139 | SAISSVSYM | 145 | DTSNLVS | 150 | QQWNTYPYT |
| | | | Y | | | | |
| | Contact | 222 | SYMYWY | 227 | LLIYDTSNLV | 229 | QQWNTYPY |
| 160 | Kabat | 132 | QASQSVSFL | 141 | DTSNLAS | 147 | QQWSTYPLT |
| | | | Y | | | | |
| | IMGT | 223 | QSVSF | — | DTS | 147 | QQWSTYPLT |
| | AbM | 132 | QASQSVSFL | 141 | DTSNLAS | 147 | QQWSTYPLT |
| | | | Y | | | | |
| | Chothia | 132 | QASQSVSFL | 141 | DTSNLAS | 147 | QQWSTYPLT |
| | | | Y | | | | |
| | Contact | 224 | SFLYWY | 228 | LLIYDTSNLA | 230 | QQWSTYPL |
| 161 | Kabat | 133 | QASSSVSFM | 141 | DTSNLAS | 147 | QQWSTYPLT |
| | | | Y | | | | |
| | IMGT | 225 | SSVSF | — | DTS | 147 | QQWSTYPLT |
| | AbM | 133 | QASSSVSFM | 141 | DTSNLAS | 147 | QQWSTYPLT |
| | | | Y | | | | |
| | Chothia | 133 | QASSSVSFM | 141 | DTSNLAS | 147 | QQWSTYPLT |
| | | | Y | | | | |
| | Contact | 226 | SFMYWY | 228 | LLIYDTSNLA | 230 | QQWSTYPL |

In some embodiments, the anti-CD122 antibody comprises a framework for grafting CDRs from another animal species. In some embodiments, CDRs from a mammalian antibody are grafted onto a human framework sequence. In some embodiments, CDRs from a mouse antibody are grafted onto a human framework sequence. In some embodiments, the human framework sequence forms part of the VH region of an antibody. In some embodiments, sequences from HCDR1, HCDR2, and HCDR3 are grafted into a human framework sequence.

In some embodiments, an anti-CD122 antibody described herein comprises an IgG framework, an IgA framework, an IgE framework, or an IgM framework. In some instances, the anti-CD122 antibody comprises an IgG framework (e.g., IgG1, IgG2, IgG3, or IgG4). In some cases, the anti-CD122 antibody comprises an IgG1 framework. In some cases, the anti-CD122 antibody comprises an IgG2 (e.g., an IgG2a or IgG2b) framework. In some cases, the anti-CD122 antibody comprises an IgG2a framework. In some cases, the anti-CD122 antibody comprises an IgG2b framework. In some cases, the anti-CD122 antibody comprises an IgG3 framework. In some cases, the anti-CD122 antibody comprises an IgG4 framework. In some embodiments, the human framework sequence is inserted into an IgG backbone to form a heavy chain sequence. In some embodiments, the IgG backbone is an IgG1 backbone, an IgG2 backbone, an IgG3 backbone, or an IgG4 backbone. In some embodiments, the IgG framework comprises a human IgG heavy chain framework sequence. In some embodiments, the human IgG heavy chain framework sequence has anti-CD122 HCDR sequences grafted into it. In some embodiments, the human IgG heavy chain framework sequence with anti-CD122 HCDR sequences grafted into it is analyzed and modeled for monoclonal antibody 3D structure to identify key amino acid positions supporting CDR loop structure. In some embodiments, key amino acid positions are identified in the human IgG heavy chain framework sequence that if reverted back to a mouse framework sequence from a mouse anti-CD122 antibody, will restore affinity to human CD122 in the context of a humanized antibody using a human IgG heavy chain framework sequence that has mouse anti-CD122 CDR sequences grafted into it. In some embodiments, those key amino acid positions are mutated in the human IgG heavy chain framework sequences back to the mouse sequences and are called back mutations. In some embodiments, the human IgG heavy chain framework sequences with mouse anti-CD122 CDR sequences grafted into it and back mutations incorporated into the framework sequence are used in a human IgG backbone to create a humanized IgG heavy chain sequence. In some embodiments, the humanized IgG heavy chain sequence is used to create an anti-CD122 antibody. In some embodiments, HCDR sequences from one of series E14-E56 are grafted into a framework sequence to create a heavy chain variable domain sequence.

In some embodiments, the anti-CD122 antibody comprises a framework for grafting CDRs from another animal species. In some embodiments, CDRs from a mammalian antibody are grafted onto a human framework sequence. In some embodiments, CDRs from a mouse antibody are grafted onto a human framework sequence. In some embodiments, the human framework sequence forms part of the VL region of an antibody. In some embodiments, sequences from LCDR1, LCDR2, and LCDR3 are grafted into a human framework sequence.

In some embodiments, an anti-CD122 antibody described herein comprises a lambda or a kappa framework. In some instances, the anti-CD122 antibody comprises a kappa framework that has CDR sequences grafted into it. In some instances, the anti-CD122 antibody comprises a kappa framework that has LCDR1, LCDR2, and LCDR3 sequences grafted into it. In some embodiments, the kappa framework forms part of a light chain. In some embodiments, the kappa framework forming part of a light chain is paired with a heavy chain described herein. In some embodiments, the kappa framework is a human kappa light chain framework. In some embodiments, the human kappa light chain framework comprises a human kappa light chain framework sequence. In some embodiments, the human kappa light chain framework sequence has anti-CD122 LCDR sequences grafted into it. In some embodiments, the human kappa light chain framework sequence with anti-CD122 LCDR sequences grafted into it is analyzed and modeled for monoclonal antibody 3D structure to identify key amino acid positions supporting CDR loop structure. In some embodiments, key amino acid positions are identified in the human kappa light chain framework sequence that if reverted back to a mouse framework sequence from a mouse anti-CD122 antibody, will restore affinity to human CD122 in the context of a humanized antibody using a human kappa light chain framework sequence that has mouse anti-CD122 CDR sequences grafted into it. In some embodiments, those key amino acid positions are mutated in the human kappa light chain framework sequences back to the mouse sequences and are called back mutations. In some embodiments, the human kappa light chain framework sequences with mouse anti-CD122 CDR sequences grafted into it and back mutations incorporated into the framework sequence are used in a human light chain backbone to create a humanized light chain sequence. In some embodiments, the humanized kappa light chain sequence is used to create an anti-CD122 antibody. In some embodiments, LCDR sequences from one of series E14-E56 are grafted into a framework sequence to create a light chain variable domain sequence.

In some embodiments, the anti-CD122 antibody comprises a VH region and a VL region in which the sequence of the VH region comprises about 80%, 85%, 90%, 95%, 96% 97%, 98%, 99%, or 100% sequence identity to SEQ ID NOs: 152-158 and the sequence of the VL region comprises about 80%, 85%, 90%, 95%, 96% 97%, 98%, 99%, or 100% sequence identity to SEQ ID NOs: 159-165. In some embodiments, the anti-CD122 antibody comprises a VH region and a VL region in which the sequence of the VH region comprises at least 95% sequence identity to SEQ ID NO: 152 and the sequence of the VL region comprises at least 95% sequence identity to SEQ ID NO: 159. In some embodiments, the anti-CD122 antibody comprises a VH region and a VL region in which the sequence of the VH region comprises at least 95% sequence identity to SEQ ID NO: 152 and the sequence of the VL region comprises at least 95% sequence identity to SEQ ID NO: 160. In some embodiments, the anti-CD122 antibody comprises a VH region and a VL region in which the sequence of the VH region comprises at least 95% sequence identity to SEQ ID NO: 152 and the sequence of the VL region comprises at least 95% sequence identity to SEQ ID NO: 161. In some embodiments, the anti-CD122 antibody comprises a VH region and a VL region in which the sequence of the VH region comprises at least 95% sequence identity to SEQ ID NO: 153 and the sequence of the VL region comprises at least 95% sequence identity to SEQ ID NO: 160. In some embodiments, the anti-CD122 antibody comprises a VH region and a VL region in which the sequence of the VH region comprises at least 95% sequence identity to SEQ ID NO: 153 and the sequence of the VL region comprises at least 95% sequence identity to SEQ ID NO: 161. In some embodiments, the anti-CD122 antibody comprises a VH region and a VL region in which the sequence of the VH region comprises at least 95% sequence identity to SEQ ID NO: 154 and the sequence of the VL region comprises at least 95% sequence identity to SEQ ID NO: 160. In some embodiments, the anti-CD122 antibody comprises a VH region and a VL region in which the sequence of the VH region comprises at least 95% sequence identity to SEQ ID NO: 154 and the sequence of the VL region comprises at least 95% sequence identity to SEQ ID NO: 161. In some embodiments, the anti-CD122 antibody comprises a VH region and a VL region in which the sequence of the VH region comprises at least 95% sequence identity to SEQ ID NO: 155 and the sequence of the VL region comprises at least 95% sequence identity to SEQ ID NO: 162. In some embodiments, the anti-CD122 antibody comprises a VH region and a VL region in which the sequence of the VH region comprises at least 95% sequence identity to SEQ ID NO: 156 and the sequence of the VL region comprises at least 95% sequence identity to SEQ ID NO: 163. In some embodiments, at least 1, 2, 3, 4, 5 or all 6 of the positions of acceptor to donor substitutions in the VH region of SEQ ID NO: 156 and the VL region of SEQ ID NO: 163 (e.g., H28, H48, H49, H68, H93, and L71) are preferably occupied by residues T, I, A, I, A, and Y respectively. In some embodiments, position H42 in the VH region of SEQ ID NO: 156 is occupied by a & In some embodiments, the anti-CD122 antibody comprises a VH region and a VL region in which the sequence of the VH region comprises at least 95% sequence identity to SEQ ID NO: 157 and the sequence of the VL region comprises at least 95% sequence identity to SEQ ID NO: 164. In some embodiments, the anti-CD122 antibody comprises a VH region and a VL region in which the sequence of the VH region comprises at least 95% sequence identity to SEQ ID NO: 158 and the sequence of the VL region comprises at least 95% sequence identity to SEQ ID NO: 165. In some embodiments, the VH region comprises a sequence selected from SEQ ID NOs: 152-158 (Table 12) and the VL region comprises a sequence selected from SEQ ID NOs: 159-165 (Table 13). VH sequences, names and correspondence SEQ ID NOs are listed in Table 12. VL sequences, names and correspondence SEQ ID NOs are listed in Table 13.

TABLE 12
VH Sequences

| SEQ ID | |
|---|---|
| NO: | VH Sequence |
| 152 | EVQLLESGGGLVQPGGSLRLSCAASGFSVTSYGVHWIRQAPGKGLEWLGVIWSGGSTDYN |
| | AAFISRLTISKDNSKNTVYFQMNSLQAEDTAIYYCARAGDYNYDGFAYWGQGTLVTVSS |
| 153 | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSS |
| 154 | EVQLLESGGGLVQPGGSLRLSCAASGFSVTSYGVHWIRQAPGKGLEWLGVIWSGGSTDYN |
| | AAFISRLTISKDNSKNTVYFQMNSLRAEDTAVYYCARAGDANYDGFAYWGQGTLVTVSS |
| 155 | EVKLVESGGGLVQPGGSLRLSCATSGFTFSDFYMEWVRQPPGKRLEWIAASRNKANDYTTE |
| | YSASVKGRFIVSRDTSQSILYLQMNALRAEDTAIYYCARSYYRYDGMDYWGQGTSVTVSS |
| 156 | EVQLVESGGGLVQPGGSLRLSCAASGFTFSDFYMEWVRQAPGKGLEWIAASRNKANDYTT |
| | EYSASVKGRFIVSRDDSKNSLYLQMNSLKTEDTAVYYCARSYYRYDGMDYWGQGTTVTV |
| | SS |
| 157 | DVQLQESGPGLVKPSQTVSLTCTVTGYSITNDNHWWNWIRQVSGSKLEWMGYIDSSGSSD |
| | NNPSLKSQISITRDTSKNQLFLQLNSVTIEDIGTYYCARGGGRDYYGMDYWGQGTSVTVSS |
| 158 | QVQLQESGPGLVKPSQTLSLTCTVSGYSITNDNHWWNWIRQHPGKGLEWMGYIDSSGSSD |
| | NNPSLKSQITISRDTSKNQLSLKLSSVTAADTAVYYCARGGGRDYYGMDYWGQGTTVTVS |
| | S |

TABLE 13
VL Sequences

| SEQ ID | |
|---|---|
| NO: | VL Sequence |
| 159 | DIVLTQSPSSLSASVGDRVTITCSGSSSVSFMYWYQQRPGKAPRLLIYDTSNLASGV |
| | PSRFSGSGSGTSYTFTISSLQPEDIATYYCQQWSTYPLTFGQGTKVEVK |
| 160 | DIQMTQSPSSLSASVGDRVTITCQASQSVSFLYWYQQRPGKAPRLLIYDTSNLASG |
| | VPSRFSGSGSGTSYTFTISSLQPEDIATYYCQQWSTYPLTFGQGTKVEIK |
| 161 | DIQMTQSPSSLSASVGDRVTITCQASSSVSFMYWYQQRPGKAPRLLIYDTSNLASG |
| | VPSRFSGSGSGTSYTFTISSLQPEDIATYYCQQWSTYPLTFGQGTKVEIK |
| 162 | QVVLTQSPVIMSASPGEKVTMTCSAISSVSYMYWYQQKPGSSPRLLIYDTSNLVSG |
| | VPVRFSGSGSGTSYSLTISRMEAEDAATYYCQQWNTYPYTFGGGTKLEIK |
| 163 | EIVLTQSPATLSLSPGERATLSCSAISSVSYMYWYQQKPGQAPRLLIYDTSNLVSGV |
| | PARFSGSGSGTDYTLTISSLEPEDFAVYYCQQWNTYPYTFGGGTKVEIK |
| 164 | DIVLTQSPASLAVSLGQRATISCRASQSVSTSSYSYVHWYQQKPGQPPKLLIKYASN |
| | LESGVPARFSGSGSGTDFTLNIHPVEEEDTATYYCQHSWDIPFTFGGGTKLEIK |
| 165 | DIVMTQSPDSLGVSLGERATINCRASQSVSTSSYSYVHWYQQKPGQPPKLLIKYAS |
| | NLESGVPDRFSGSGSGTDFTLTISSLQAEDVAVYYCQHSWDIPFTFGQGTKLEIK |

In some embodiments, the anti-CD122 antibody comprises a VH region comprising SEQ ID NO: 152 and a VL region comprising SEQ ID NO: 159. In some embodiments, the anti-CD 122 antibody comprises a VH region comprising SEQ ID NO: 152 and a VL region comprising SEQ ID NO: 160. In some embodiments, the anti-CD122 antibody comprises a VH region comprising SEQ ID NO: 152 and a VL region comprising SEQ ID NO: 161. In some embodiments, the anti-CD122 antibody comprises a VH region comprising SEQ ID NO: 153 and a VL region comprising SEQ ID NO: 160. In some embodiments, the anti-CD122 antibody comprises a VH region comprising SEQ ID NO: 153 and a VL region comprising SEQ ID NO: 161. In some embodiments, the anti-CD122 antibody comprises a VH region comprising SEQ ID NO: 154 and a VL region comprising SEQ ID NO: 160. In some embodiments, the anti-CD122 antibody comprises a VH region comprising SEQ ID NO: 154 and a VL region comprising SEQ ID NO: 161. In some embodiments, the anti-CD122 antibody comprises a VH region comprising SEQ ID NO: 155 and a VL region comprising SEQ ID NO: 162. In some embodiments, the anti-CD122 antibody comprises a VH region comprising SEQ ID NO: 156 and a VL region comprising SEQ ID NO: 163. In some embodiments, the anti-CD122 antibody comprises a VH region comprising SEQ ID NO: 157 and a VL region comprising SEQ ID NO: 164. In some embodiments, the anti-CD122 antibody comprises a VH region comprising SEQ ID NO: 158 and a VL region comprising SEQ ID NO: 165.

In some embodiments, an anti-CD122 antibody described herein is a full-length antibody. In other aspects, the anti-CD122 antibody is an antigen-binding fragment thereof. In some cases, the anti-CD122 antibody is a humanized antibody or an antigen-binding fragment thereof, a chimeric antibody or an antigen-binding fragment thereof, a monoclonal antibody or an antigen-binding fragment thereof. In some cases, the anti-CD122 antibody is monovalent Fab′, F(ab)′3 fragments, single-chain variable fragment (scFv), (scFv)2, minibody, disulfide stabilized Fv protein (“dsFv”), single-domain antibody (sdAb), Ig NAR, camelid antibody or an antigen-binding fragment thereof, or a chemically-modified derivative thereof. In some embodiments, the antibody comprises an Immunoglobulin G (IgG) antibody or variant thereof. In some embodiments, the IgG antibody or variant thereof comprises an IgG1 antibody or variant thereof. In some embodiments, the IgG antibody or variant thereof comprises an IgG2 antibody or variant thereof. In some embodiments, the IgG antibody or variant thereof comprises an IgG3 antibody or variant thereof. In some embodiments, the IgG antibody or variant thereof comprises an IgG4 antibody or variant thereof. In some embodiments, the antibody comprises an IgA antibody. In some embodiments, the antibody comprises an IgM antibody. In some embodiments, the antibody comprises an IgE antibody.

In some cases, an anti-CD122 antibody comprises one or more mutations in a framework region, e.g., in the CH1 domain, CH2 domain, CH3 domain, hinge region, or a combination thereof. In some instances, the one or more mutations are to stabilize the antibody. In some instances, the one or more mutations are to increase half-life. In some instances, the one or more mutations are to modulate Fc receptor interactions. In some instances, the one or more mutations are to reduce or eliminate Fc effector functions such as FcγR, antibody-dependent cell-mediated cytotoxicity (ADCC), or complement-dependent cytotoxicity (CDC). In additional instances, the one or more mutations are to modulate glycosylation.

In some embodiments, the one or more mutations are located in the Fc region.

In some embodiments, the human IgG constant region is modified to alter antibody-dependent cellular cytotoxicity (ADCC) and/or complement-dependent cytotoxicity (CDC), e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Urban et al., 2021 Front Immunol. November 25; 12:724361; Zhou et al., 2020 MAbs. January-December; 12(1):1814583; Reviewed in Kaneko and Niwa, 2011 Biodrugs, 25(1): 1-11.

In some embodiments, the Fc region of an anti-CD122 antibody heavy chain is categorized as IgG1, IgG2, or IgG4. In some embodiments, the IgG1, IgG2, or IgG4 is human IgG1, human IgG2, or human IgG4. In some embodiments, the IgG1, IgG2, or IgG4 is mouse IgG1, mouse IgG2, or mouse IgG4. In some embodiments, the IgG1, IgG2, or IgG4 is rat IgG1, rat IgG2, or rat IgG4. In some embodiments, the Fc region of the anti-CD122 antibody heavy chain is human IgG4 wild-type. In some embodiments, the Fc region of the anti-CD122 antibody heavy chain is human IgG1 wild-type. In some embodiments, the Fc region of the anti-CD122 antibody heavy chain is human IgG1 wild-type having a REEM allotype sequence. In some embodiments, the Fc region of the anti-CD122 antibody heavy chain is human IgG2 wild-type. In some embodiments, the Fc region of the anti-CD122 antibody heavy chain is human IgG1-3M wild-type. In some embodiments, the Fc region of the anti-CD122 antibody heavy chain is human IgG1-3M wild-type having a REEM allotype sequence. In some embodiments, the Fc region of the anti-CD122 antibody heavy chain is human IgG4 having a S228P substitution. In some embodiments, the Fc region of the anti-CD122 antibody heavy chain is human IgG1 having a L234A substitution. In some embodiments, the Fc region of the anti-CD122 antibody heavy chain is human IgG1 having a L235A substitution. In some embodiments, the Fc region of the anti-CD122 antibody heavy chain is human IgG1 having L234A and L235A substitutions. In some embodiments, the Fc region of the anti-CD122 antibody heavy chain is human IgG1 having L234A, L235A, and P329G substitutions. In some embodiments, the Fc region of the anti-CD122 antibody heavy chain is human IgG1 wild-type having a REEM allotype sequence and L234A, L235A, and P329G substitutions. Representative Fc region sequences used in anti-CD122 antibody heavy chains described herein are listed in Table 14. In some embodiments, the human Fc region of the anti-CD122 antibody heavy chain comprises an amino acid sequence selected from SEQ ID NO: 166-175.

TABLE 14
Fc regions

| Fc | SEQ | |
|---|---|---|
| portion | ID | |
| name | NO: | Sequence |
| Human | 166 | ASTKGPSVFPLAPCSRSTSESTAALGCLVKDYFPEPVTVSWNSGALTSGVHTF |
| IgG4 | | PAVLQSSGLYSLSSVVTVPSSSLGTKTYTCNVDHKPSNTKVDKRVESKYGPP |
| wild-type | | CPSCPAPEFLGGPSVFLFPPKPKDTLMISRTPEVTCVVVDVSQEDPEVQFNW |
| | | YVDGVEVHNAKTKPREEQFNSTYRVVSVLTVLHQDWLNGKEYKCKVSNK |
| | | GLPSSIEKTISKAKGQPREPQVYTLPPSQEEMTKNQVSLTCLVKGFYPSDIAV |
| | | EWESNGQPENNYKTTPPVLDSDGSFFLYSRLTVDKSRWQEGNVFSCSVMHE |
| | | ALHNHYTQKSLSLSLGK |
| Human | 167 | ASTKGPSVFPLAPCSRSTSESTAALGCLVKDYFPEPVTVSWNSGALTSGVHTF |
| IgG4 | | PAVLQSSGLYSLSSVVTVPSSSLGTKTYTCNVDHKPSNTKVDKRVESKYGPP |
| (S228P) | | CPPCPAPEFLGGPSVFLFPPKPKDTLMISRTPEVTCVVVDVSQEDPEVQFNWY |
| | | VDGVEVHNAKTKPREEQFNSTYRVVSVLTVLHQDWLNGKEYKCKVSNKGL |
| | | PSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEAL |
| | | HNHYTQKSLSLSLGK |
| Human | 168 | ASTKGPSVFPLAPSSKSTSGGTAALGCLVKDYFPEPVTVSWNSGALTSGVHTF |
| IgG1 | | PAVLQSSGLYSLSSVVTVPSSSLGTQTYICNVNHKPSNTKVDKKVEPKSCDKT |
| wild-type | | HTCPPCPAPELLGGPSVFLFPPKPKDTLMISRTPEVTCVVVDVSHEDPEVKFNW |
| | | YVDGVEVHNAKTKPREEQYNSTYRVVSVLTVLHQDWLNGKEYKCKVSNKAL |
| | | PAPIEKTISKAKGQPREPQVYTLPPSRDELTKNQVSLTCLVKGFYPSDIAVEWES |
| | | NGQPENNYKTTPPVLDSDGSFFLYSKLTVDKSRWQQGNVFSCSVMHEALHNH |
| | | YTQKSLSLSPGK |
| Human | 169 | ASTKGPSVFPLAPSSKSTSGGTAALGCLVKDYFPEPVTVSWNSGALTSGVHTF |
| IgG1 | | PAVLQSSGLYSLSSVVTVPSSSLGTQTYICNVNHKPSNTKVDKKVEPKSCDKT |
| LALA | | HTCPPCPAPEAAGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSN |
| | | KALPAPIEKTISKAKGQPREPQVYTLPPSRDELTKNQVSLTCLVKGFYPSDIAVE |
| | | WESNGQPENNYKTTPPVLDSDGSFFLYSKLTVDKSRW |
| | | QQGNVFSCSVMHEALHNHYTQKSLSLSPGK |
| Human | 170 | ASTKGPSVFPLAPSSKSTSGGTAALGCLVKDYFPEPVTVSWNSGALTSGVHT |
| IgG1 | | FPAVLQSSGLYSLSSVVTVPSSSLGTQTYICNVNHKPSNTKVDKKVEPKSCDK |
| LALA | | THTCPPCPAPEAAGGPSVFLFPPKPKDTLMISRTPEVTCVVVDVSHEDPEVKFN |
| P329G | | WYVDGVEVHNAKTKPREEQYNSTYRVVSVLTVLHQDWLNGKEYKCKVSNK |
| | | ALGAPIEKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVMHEALHN |
| | | HYTQKSLSLSPGK |
| Human | 171 | ASTKGPSVFPLAPSSKSTSGGTAALGCLVKDYFPEPVTVSWNSGALTSGVHTF |
| IgG-3M | | PAVLQSSGLYSLSSVVTVPSSSLGTQTYICNVNHKPSNTKVDKKVEPKSCDKT |
| | | HTCPPCPAPEAAGAPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNK |
| | | ALPAPIEKTISKAKGQPREPQVYTLPPSRDELTKNQVSLTCLVKGFYPSDIAVE |
| | | WESNGQPENNYKTTPPVLDSDGSFFLYSKLTVDKSRWQQGNVFSCSVMHEAL |
| | | HNHYTQKSLSLSPGK |
| Human | 172 | ASTKGPSVFPLAPCSRSTSESTAALGCLVKDYFPEPVTVSWNSGALTSGVHTFP |
| IgG2 | | AVLQSSGLYSLSSVVTVPSSNFGTQTYTCNVDHKPSNTKVDKTVERKCCVECP |
| wild-type | | PCPAPPVAGPSVFLFPPKPKDTLMISRTPEVTCVVVDVSHEDPEVQFNWYVDG |
| | | VEVHNAKTKPREEQFNSTFRVVSVLTVVHQDWLNGKEYKCKVSNKGLPAPIE |
| | | KTISKTKGQPREPQVYTLPPSREEMTKNQVSLTCLVKGFYPSDISVEWESNGQP |
| | | ENNYKTTPPMLDSDGSFFLYSKLTVDKSRWQQGNVFSCSVMHEALHNHYTQK |
| | | SLSLSPGK |
| Human | 173 | ASTKGPSVFPLAPSSKSTSGGTAALGCLVKDYFPEPVTVSWNSGALTSGVHT |
| IgG1 | | FPAVLQSSGLYSLSSVVTVPSSSLGTQTYICNVNHKPSNTKVDKKVEPKSCDK |
| wild-type | | THTCPPCPAPELLGGPSVFLFPPKPKDTLMISRTPEVTCVVVDVSHEDPEVKFN |
| REEM | | WYVDGVEVHNAKTKPREEQYNSTYRVVSVLTVLHQDWLNGKEYKCKVSNK |
| allotype | | ALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEAL |
| | | HNHYTQKSLSLSPGK |
| Human | 174 | ASTKGPSVFPLAPSSKSTSGGTAALGCLVKDYFPEPVTVSWNSGALTSGVHTF |
| IgG1 | | PAVLQSSGLYSLSSVVTVPSSSLGTQTYICNVNHKPSNTKVDKKVEPKSCDKT |
| wild-type | | HTCPPCPAPEAAGGPSVFLFPPKPKDTLMISRTPEVTCVVVDVSHEDPEVKFN |
| REEM | | WYVDGVEVHNAKTKPREEQYNSTYRVVSVLTVLHQDWLNGKEYKCKVSN |
| allotype | | KALGAPIEKTISKAKGQPREPQVYTLPPSREEMTKNQVSLTCLVKGFYPSDIA |
| LALA | | VEWESNGQPENNYKTTPPVLDSDGSFFLYSKLTVDKSRWQQGNVFSCSVMH |
| P329G | | EALHNHYTQKSLSLSPGK |
| Human | 175 | ASTKGPSVFPLAPSSKSTSGGTAALGCLVKDYFPEPVTVSWNSGALTSGVHTF |
| IgG1-3M | | PAVLQSSGLYSLSSVVTVPSSSLGTQTYICNVNHKPSNTKVDKKVEPKSCDKT |
| REEM | | HTCPPCPAPEAAGAPSVFLFPPKPKDTLMISRTPEVTCVVVDVSHEDPEVKFN |
| allotype | | WYVDGVEVHNAKTKPREEQYNSTYRVVSVLTVLHQDWLNGKEYKCKVSNK |
| | | ALPAPIEKTISKAKGQPREPQVYTLPPSREEMKNQVSLTCLVKGFYPSDIAVEW |
| | | ESNGQPENNYKTTPPVLDSDGSFFLYSKLTVDKSRWQQGNVFSCSVMHEALH |
| | | NHYTQKSLSLSPGK |

In some embodiments, the anti-CD122 antibodies light chains comprise a light chain constant regions. In some embodiments, the light chain constant region is a kappa light chain constant region. In some embodiments, the light chain constant region is a lambda light chain constant region. In some embodiments, the light chain constant region is a human kappa light chain constant region. In some embodiments, the light chain constant region is a human lambda light chain constant region. In some embodiments, the light chain constant region is a mouse kappa light chain constant region. In some embodiments, the light chain constant region is a mouse lambda light chain constant region. In some embodiments, the light chain constant region is a rat kappa light chain constant region. In some embodiments, the light chain constant region is a rat lambda light chain constant region. In some embodiments, the human kappa light chain constant region is listed in Table 15. In some embodiments, the human kappa light chain constant region comprises an amino acid sequence of SEQ ID NO: 176.

TABLE 15
Light chain constant region

| | SEQ | |
|---|---|---|
| Light chain | ID | |
| constant | NO: | Sequence |
| Human kappa | 176 | RTVAAPSVFIFPPSDEQLKSGTASVVCLLNNFYPREAKVQWKVDNALQ |
| light chain | | SGNSQESVTEQDSKDSTYSLSSTLTLSKADYEKHKVYACEVTHQGLSSP |
| constant | | VTKSFNRGEC |

In some embodiments, an anti-CD122 antibody described herein is a full-length antibody, comprising a heavy chain (HC) and a light chain (LC). In some embodiments, the HC comprises about 80%, 85%, 90%, 95%, 96% 97%, 98%, 99%, or 100% sequence identity to SEQ ID NOs: 177-196. In some embodiments, the HC comprises at least 80%, at least 85%, at least 90%, or at least 95% sequence identity sequence identity to SEQ ID NOs: 177-196. In some embodiments, the LC comprises about 80%, 85%, 90%, 95%, 96% 97%, 98%, 99%, or 100% sequence identity to SEQ ID NOs: 197-199. In some embodiments, the LC comprises at least 80%, at least 85%, at least 90%, or at least 95% sequence identity sequence identity to SEQ ID NOs: 197-199. In some cases, the heavy chain (HC) comprises a sequence selected from Table 16. In some cases, the light chain (LC) comprises a sequence selected from Table 17. In some cases, a HC comprising a sequence from Table 16 and a LC comprising a sequence from Table 17 are paired together to form an anti-CD122 antibody.

TABLE 16
anti-CD122 antibody heavy chain sequence

| SEQ ID | |
|---|---|
| NO: | Heavy Chain sequence |
| 177 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSS |
| | ASTKGPSVFPLAPCSRSTSESTAALGCLVKDYFPEPVTVSWNSGALTSGVHTFPAVLQSSGLY |
| | SLSSVVTVPSSSLGTKTYTCNVDHKPSNTKVDKRVESKYGPPCPSCPAPEFLGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLGK* |
| 178 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPCSRSTSESTAALGCLVKDYFPEPVTVSWNSGALTSGVHTFPAVLQSSGLYSL |
| | SSVVTVPSSSLGTKTYTCNVDHKPSNTKVDKRVESKYGPPCPPCPAPEFLGGPSVFLFPPKPK |
| | DTLMISRTPEVTCVVVDVSQEDPEVQFNWYVDGVEVHNAKTKPREEQFNSTYRVVSVLTVL |
| | HQDWLNGKEYKCKVSNKGLPSSIEKTISKAKGQPREPQVYTLPPSQEEMTKNQVSLTCLVKG |
| | FYPSDIAVEWESNGQPENNYKTTPPVLDSDGSFFLYSRLTVDKSRWQEGNVFSCSVMHEALH |
| | NHYTQKSLSLSLGK* |
| 179 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPELLGGPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQGNVFSCSV |
| | MHEALHNHYTQKSLSLSPGK* |
| 180 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPEAAGGPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKSRWQQGNVFSCSVM |
| | HEALHNHYTQKSLSLSPGK* |
| 181 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPEAAGGPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLPPSRDELTKNQVSLTC |
| | LVKGFYPSDIAVEWESNGQPENNYKTTPPVLDSDGSFFLYSKLTVDKSRWQQGNVFSCSVM |
| | HEALHNHYTQKSLSLSPGK* |
| 182 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPEAAGAPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSRDELTKNQVSLTC |
| | LVKGFYPSDIAVEWESNGQPENNYKTTPPVLDSDGSFFLYSKLTVDKSRWQQGNVFSCSVM |
| | HEALHNHYTQKSLSLSPGK* |
| 183 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPCSRSTSESTAALGCLVKDYFPEPVTVSWNSGALTSGVHTFPAVLQSSGLYSL |
| | SSVVTVPSSNFGTQTYTCNVDHKPSNTKVDKTVERKCCVECPPCPAPPVAGPSVFLFPPKPKD |
| | TLMISRTPEVTCVVVDVSHEDPEVQFNWYVDGVEVHNAKTKPREEQFNSTFRVVSVLTVVH |
| | QDWLNGKEYKCKVSNKGLPAPIEKTISKTKGQPREPQVYTLPPSREEMTKNQVSLTCLVKGF |
| | YPSDISVEWESNGQPENNYKTTPPMLDSDGSFFLYSKLTVDKSRWQQGNVFSCSVMHEALH |
| | NHYTQKSLSLSPGK* |
| 184 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPELLGGPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMTKNQVSLTC |
| | LVKGFYPSDIAVEWESNGQPENNYKTTPPVLDSDGSFFLYSKLTVDKSRWQQGNVFSCSVM |
| | HEALHNHYTQKSLSLSPGK* |
| 185 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPEAAGGPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALGAPIEKTISKAKGQPREPQVYTLPPSREEMTKNQVSLT |
| | CLVKGFYPSDIAVEWESNGQPENNYKTTPPVLDSDGSFFLYSKLTVDKSRWQQGNVFSCSV |
| | MHEALHNHYTQKSLSLSPGK* |
| 186 | (X)ₙ₌₁₄₋₄₀ |
| | EVQLLESGGGLVQPGGSLRLSCAASGFTVTSYAVHWVRQAPGKGLEWLGVIWSGGSTDYN |
| | AAFISRLTISKDNSKNTVYFQMNSLRAEDTAVYYCARAGDANYDGFAYWGQGTLVTVSSAS |
| | TKGPSVFPLAPSSKSTSGGTAALGCLVKDYFPEPVTVSWNSGALTSGVHTFPAVLQSSGLYSL |
| | SSVVTVPSSSLGTQTYICNVNHKPSNTKVDKKVEPKSCDKTHTCPPCPAPEAAGAPSVFLFPP |
| | KPKDTLMISRTPEVTCVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLPPSREEMKNQVSLTCL |
| | VKGFYPSDIAVEWESNGQPENNYKTTPPVLDSDGSFFLYSKLTVDKSRWQQGNVFSCSVMH |
| | EALHNHYTQKSLSLSPGK* |
| 187 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPCSRSTSESTAALGCLVKDYFPEPVTVSWNSGALTSGVHTFPAVLQSSGLY |
| | SLSSVVTVPSSSLGTKTYTCNVDHKPSNTKVDKRVESKYGPPCPSCPAPEFLGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLGK* |
| 188 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPCSRSTSESTAALGCLVKDYFPEPVTVSWNSGALTSGVHTFPAVLQSSGLY |
| | SLSSVVTVPSSSLGTKTYTCNVDHKPSNTKVDKRVESKYGPPCPPCPAPEFLGGPSVFLFPPKP |
| | KDTLMISRTPEVTCVVVDVSQEDPEVQFNWYVDGVEVHNAKTKPREEQFNSTYRVVSVLTV |
| | LHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSRLTVDKSRWQEGNVFSCSVMHEAL |
| | HNHYTQKSLSLSLGK* |
| 189 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPELLGGPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQGNVFSCSV |
| | MHEALHNHYTQKSLSLSPGK* |
| 190 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPEAAGGPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQGNVFSCSV |
| | MHEALHNHYTQKSLSLSPGK* |
| 191 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDK*THTCPPCPAPEAAGGPSVF |
| | LFPPKPKDTLMISRTPEVTCVVVDVSHEDPEVKFNWYVDGVEVHNAKTKPREEQYNSTYRV |
| | VSVLTVLHQDWLNGKEYKCKVSNKALGAPIEKTISKAKGQPREPQVYTLPPSRDELTKNQVS |
| | LTCLVKGFYPSDIAVEWESNGQPENNYKTTPPVLDSDGSFFLYSKLTVDKSRWQQGNVFSCS |
| | VMHEALHNHYTQKSLSLSPGK* |
| 192 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPEAAGAPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDELTKNQVSL |
| | TCLVKGFYPSDIAVEWESNGQPENNYKTTPPVLDSDGSFFLYSKLTVDKSRWQQGNVFSCSV |
| | MHEALHNHYTQKSLSLSPGK* |
| 193 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPCSRSTSESTAALGCLVKDYFPEPVTVSWNSGALTSGVHTFPAVLQSSGLY |
| | SLSSVVTVPSSNFGTQTYTCNVDHKPSNTKVDKTVERKCCVECPPCPAPPVAGPSVFLFPPKP |
| | KDTLMISRTPEVTCVVVDVSHEDPEVQFNWYVDGVEVHNAKTKPREEQFNSTFRVVSVLTV |
| | VHQDWLNGKEYKCKVSNKGLPAPIEKTISKTKGQPREPQVYTLPPSREEMTKNQVSLTCLVK |
| | GFYPSDISVEWESNGQPENNYKTTPPMLDSDGSFFLYSKLTVDKSRWQQGNVFSCSVMHEA |
| | LHNHYTQKSLSLSPGK* |
| 194 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPELLGGPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSREEMTKNQVSL |
| | TCLVKGFYPSDIAVEWESNGQPENNYKTTPPVLDSDGSFFLYSKLTVDKSRWQQGNVFSCSV |
| | MHEALHNHYTQKSLSLSPGK* |
| 195 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPEAAGGPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALGAPIEKTISKAKGQPREPQVYTLPPSREEMTKNQVS |
| | LTCLVKGFYPSDIAVEWESNGQPENNYKTTPPVLDSDGSFFLYSKLTVDKSRWQQGNVFSCS |
| | VMHEALHNHYTQKSLSLSPGK* |
| 196 | (X)ₙ₌₁₄₋₄₀ |
| | EVQLVESGGGLVQPGGSLRLSCAASGFTFSDFYMEWVRQAPGKGLEWIAASRNKANDYTTE |
| | YSASVKGRFIVSRDDSKNSLYLQMNSLKTEDTAVYYCARSYYRYDGMDYWGQGTTVTVSS |
| | ASTKGPSVFPLAPSSKSTSGGTAALGCLVKDYFPEPVTVSWNSGALTSGVHTFPAVLQSSGL |
| | YSLSSVVTVPSSSLGTQTYICNVNHKPSNTKVDKKVEPKSCDKTHTCPPCPAPEAAGAPSVFL |
| | FPPKPKDTLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSREEMKNQVSLT |
| | CLVKGFYPSDIAVEWESNGQPENNYKTTPPVLDSDGSFFLYSKLTVDKSRWQQGNVFSCSV |
| | MHEALHNHYTQKSLSLSPGK* |

(X)ₙ₌₁₄₋₄₀ is a leader sequence is a leader sequence

TABLE 17
anti-CD122 antibody light chain sequence

| SEQ ID | |
|---|---|
| NO: | Light Chain sequence |
| 197 | (X)ₙ₌₁₄₋₄₀ |
| | DIQMTQSPSSLSASVGDRVTITCQASQSVSFLYWYQQRPGKAPRLLIYDTSNLAS |
| | GVPSRFSGSGSGTSYTFTISSLQPEDIATYYCQQWSTYPLTFGQGTKVEIKRTVA |
| | APSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC* |
| 198 | (X)ₙ₌₁₄₋₄₀ |
| | DIQMTQSPSSLSASVGDRVTITCQASSSVSFMYWYQQRPGKAPRLLIYDTSNLA |
| | SGVPSRFSGSGSGTSYTFTISSLQPEDIATYYCQQWSTYPLTFGQGTKVEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |
| 199 | (X)ₙ₌₁₄₋₄₀ |
| | EIVLTQSPATLSLSPGERATLSCSAISSVSYMYWYQQKPGQAPRLLIYDTSNLVS |
| | GVPARFSGSGSGTDYTLTISSLEPEDFAVYYCQQWNTYPYTFGGGTKVEIKRTV |
| | AAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC* |

(X)ₙ₌₁₄₋₄₀ is a leader sequence

In some embodiments, an anti-CD122 antibody described herein is a full-length antibody, comprising a heavy chain (HC) and a light chain (LC). In some embodiments, the full-length antibody comprises the HC comprising SEQ ID NO: 177 and the LC comprising SEQ ID NO: 197. In some embodiments, the full-length antibody comprises the HC comprising SEQ ID NO: 178 and the LC comprising SEQ ID NO: 197. In some embodiments, the full-length antibody comprises the HC comprising SEQ ID NO: 179 and the LC comprising SEQ ID NO: 197. In some embodiments, the full-length antibody comprises the HC comprising SEQ ID NO: 180 and the LC comprising SEQ ID NO: 197. In some embodiments, the full-length antibody comprises the HC comprising SEQ ID NO: 181 and the LC comprising SEQ ID NO: 197. In some embodiments, the full-length antibody comprises the HC comprising SEQ ID NO: 182 and the LC comprising SEQ ID NO: 197. In some embodiments, the full-length antibody comprises the HC comprising SEQ ID NO: 183 and the LC comprising SEQ ID NO: 197. In some embodiments, the full-length antibody comprises the HC comprising SEQ ID NO: 184 and the LC comprising SEQ ID NO: 197. In some embodiments, the full-length antibody comprises the HC comprising SEQ ID NO: 185 and the LC comprising SEQ ID NO: 197. In some embodiments, the full-length antibody comprises the HC comprising SEQ ID NO: 186 and the LC comprising SEQ ID NO: 197.

In some embodiments, the full-length antibody comprises the HC comprising SEQ ID NO: 177 and the LC comprising SEQ ID NO: 198. In some embodiments, the full-length antibody comprises the HC comprising SEQ ID NO: 178 and the LC comprising SEQ ID NO: 198. In some embodiments, the full-length antibody comprises the HC comprising SEQ ID NO: 179 and the LC comprising SEQ ID NO: 198. In some embodiments, the full-length antibody comprises the HC comprising SEQ ID NO: 180 and the LC comprising SEQ ID NO: 198. In some embodiments, the full-length antibody comprises the HC comprising SEQ ID NO: 181 and the LC comprising SEQ ID NO: 198. In some embodiments, the full-length antibody comprises the HC comprising SEQ ID NO: 182 and the LC comprising SEQ ID NO: 198. In some embodiments, the full-length antibody comprises the HC comprising SEQ ID NO: 183 and the LC comprising SEQ ID NO: 198. In some embodiments, the full-length antibody comprises the HC comprising SEQ ID NO: 184 and the LC comprising SEQ ID NO: 198. In some embodiments, the full-length antibody comprises the HC comprising SEQ ID NO: 185 and the LC comprising SEQ ID NO: 198. In some embodiments, the full-length antibody comprises the HC comprising SEQ ID NO: 186 and the LC comprising SEQ ID NO: 198.

In some embodiments, the full-length antibody comprises the HC comprising SEQ ID NO: 187 and the LC comprising SEQ ID NO: 199. In some embodiments, the full-length antibody comprises the HC comprising SEQ ID NO: 188 and the LC comprising SEQ ID NO: 199. In some embodiments, the full-length antibody comprises the HC comprising SEQ ID NO: 189 and the LC comprising SEQ ID NO: 199. In some embodiments, the full-length antibody comprises the HC comprising SEQ ID NO: 190 and the LC comprising SEQ ID NO: 199. In some embodiments, the full-length antibody comprises the HC comprising SEQ ID NO: 191 and the LC comprising SEQ ID NO: 199. In some embodiments, the full-length antibody comprises the HC comprising SEQ ID NO: 192 and the LC comprising SEQ ID NO: 199. In some embodiments, the full-length antibody comprises the HC comprising SEQ ID NO: 193 and the LC comprising SEQ ID NO: 199. In some embodiments, the full-length antibody comprises the HC comprising SEQ ID NO: 194 and the LC comprising SEQ ID NO: 199. In some embodiments, the full-length antibody comprises the HC comprising SEQ ID NO: 195 and the LC comprising SEQ ID NO: 199. In some embodiments, the full-length antibody comprises the HC comprising SEQ ID NO: 196 and the LC comprising SEQ ID NO: 199.

In some embodiments, the anti-CD122 antibody heavy chain and the anti-CD122 antibody light chain have a leader sequence near an amino-terminal region of each polypeptide. In some embodiments, the leader sequence is N-terminal to an anti-CD122 antibody heavy chain sequence. In some embodiments, the leader sequence is N-terminal to an anti-CD122 antibody heavy chain sequence provided in Table 16. In some embodiments, the leader sequence is N-terminal to an anti-CD122 antibody light chain sequence. In some embodiments, the leader sequence is N-terminal to an anti-CD122 antibody light chain sequence provided in Table 17. In some embodiments, (X)ₙ₌₁₄₋₄₀ is used to represent amino acids of a leader sequence at the amino-terminal end of an anti-CD122 antibody heavy chain sequence. In some embodiments, (X)ₙ₌₁₄₋₄₀ is used to represent amino acids of a leader sequence at the amino-terminal end of an anti-CD122 antibody light chain sequence. In some embodiments, the leader sequence is a length of between 14-40 amino acids. In some embodiments, the leader sequence is a length of between 15-35 amino acids. In some embodiments, the leader sequence of an anti-CD122 antibody heavy chain and an anti-CD122 antibody light chain paired to form an anti-CD122 antibody are identical to each other. In some embodiments, the leader sequence of an anti-CD122 antibody heavy chain and an anti-CD122 antibody light chain paired to form an anti-CD122 antibody are not identical to each other. In some embodiments, the leader sequence comprises an amino acid sequence listed in Table 8. In some embodiments, the leader sequence comprises an amino acid sequence listed in Table 18. In some embodiments, the anti-CD122 antibody heavy chain contains an additional amino acid sequence N-terminal to the leader sequence. In some embodiments, the anti-CD122 antibody heavy chain contains an additional amino acid sequence immediately after the leader sequence. In some embodiments, the additional amino acid sequence immediately after the leader sequence is before a start of a heavy chain variable domain. In some embodiments, the anti-CD122 antibody light chain contains an additional amino acid sequence N-terminal to the leader sequence. In some embodiments, the anti-CD122 antibody light chain contains an additional amino acid sequence immediately after the leader sequence. In some embodiments, the additional amino acid sequence immediately after the leader sequence is before a start of a light chain variable domain. In some embodiments, the leader sequence used N-terminal to the anti-CD122 antibody heavy chain sequence comprises a leader sequence comprising SEQ ID NO: 106. In some embodiments, the leader sequence used N-terminal to the anti-CD122 antibody heavy chain sequence comprises a leader sequence comprising SEQ ID NO: 107. In some embodiments, the leader sequence used N-terminal to the anti-CD122 antibody heavy chain sequence comprises a leader sequence comprising SEQ ID NO: 108. In some embodiments, the leader sequence used N-terminal to the anti-CD122 antibody heavy chain sequence comprises a leader sequence comprising SEQ ID NO: 109. In some embodiments, the leader sequence used N-terminal to the anti-CD122 antibody heavy chain sequence comprises a leader sequence comprising SEQ ID NO: 200. In some embodiments, the leader sequence used N-terminal to the anti-CD122 antibody light chain sequence comprises a leader sequence comprising SEQ ID NO:106. In some embodiments, the leader sequence used N-terminal to the anti-CD122 antibody light chain sequence comprises a leader sequence comprising SEQ ID NO: 107. In some embodiments, the leader sequence used N-terminal to the anti-CD122 antibody light chain sequence comprises a leader sequence comprising SEQ ID NO: 108. In some embodiments, the leader sequence used N-terminal to the anti-CD122 antibody light chain sequence comprises a leader sequence comprising SEQ ID NO: 109. In some embodiments, the leader sequence used N-terminal to the anti-CD122 antibody light chain sequence comprises a leader sequence comprising SEQ ID NO:201.

In some embodiments, an HC peptide and an LC peptide are paired together to form an anti-CD122 antibody or an anti-CD122 antibody fragment. In some embodiments, two heavy chains are linked to each other by disulfide bonds and each heavy chain is linked to a light chain by a disulfide bond. In some embodiments, the particular HC peptides and particular LC peptides paired together to form an anti-CD122 antibody are named from G1-G50 according to Table 19. In some embodiments, G1 is a chimeric anti-CD122 antibody with mouse parental variable domain sequences in a human IgG1 backbone. In some embodiments, G2-10 are humanized anti-CD122 antibodies with mouse-derived CDRs incorporated into human variable domain framework sequences that have been optimized to retain affinity to CD122 in a human IgG1 backbone. In some embodiments, G11 is a chimeric anti-CD122 antibody with mouse parental variable domain sequences in a human IgG4 backbone. In some embodiments, G12-20 are humanized anti-CD122 antibodies with mouse-derived CDRs incorporated into human variable domain framework sequences that have been optimized to retain affinity to CD122 in a human IgG4 backbone. In some embodiments, an antibody selected from G1-G50 is used in a method of prophylaxis or treatment of aGvHD. In some embodiments, an antibody selected from G1-G50 is used in a method of prophylaxis or treatment of cGvHD. In some exemplary embodiments, a rat anti-mouse IL15Rβ monoclonal antibody is used in a method of prophylaxis or treatment of cGvHD. In some embodiments, the rat anti-mouse IL15Rβ monoclonal antibody is clone TM-β1. In some embodiments, G21-50 are humanized anti-CD122 antibodies with mouse-derived CDRs incorporated into human variable domain framework sequences that have been optimized to retain affinity to CD122 in a human IgG1 backbone.

TABLE 18
Leader Sequences for use with heavy chains and
light chains

| SEQ ID NO: | Leader Sequence |
|---|---|
| 200 | MKLWLNWVFLLTLLHGIQC |
| 201 | MDFQVQIFSFLLISASVIVSRG |

TABLE 19
Antibody names with heavy chain/light
chain sequence combinations

| Antibody | HC peptide | LC peptide |
|---|---|---|
| name | SEQ ID NO: | SEQ ID NO: |
| G1 | 94 | 102 |
| G2 | 95 | 103 |
| G3 | 95 | 104 |
| G4 | 95 | 105 |
| G5 | 96 | 103 |
| G6 | 96 | 104 |
| G7 | 96 | 105 |
| G8 | 97 | 103 |
| G9 | 97 | 104 |
| G10 | 97 | 105 |
| G11 | 98 | 102 |
| G12 | 99 | 103 |
| G13 | 99 | 104 |
| G14 | 99 | 105 |
| G15 | 100 | 103 |
| G16 | 100 | 104 |
| G17 | 100 | 105 |
| G18 | 101 | 103 |
| G19 | 101 | 104 |
| G20 | 101 | 105 |
| G21 | 177 | 197 |
| G22 | 178 | 197 |
| G23 | 179 | 197 |
| G24 | 180 | 197 |
| G25 | 181 | 197 |
| G26 | 182 | 197 |
| G27 | 183 | 197 |
| G28 | 184 | 197 |
| G29 | 185 | 197 |
| G30 | 186 | 197 |
| G31 | 177 | 198 |
| G32 | 178 | 198 |
| G33 | 179 | 198 |
| G34 | 180 | 198 |
| G35 | 181 | 198 |
| G36 | 182 | 198 |
| G37 | 183 | 198 |
| G38 | 184 | 198 |
| G39 | 185 | 198 |
| G40 | 186 | 198 |
| G41 | 187 | 199 |
| G42 | 188 | 199 |
| G43 | 189 | 199 |
| G44 | 190 | 199 |
| G45 | 191 | 199 |
| G46 | 192 | 199 |
| G47 | 193 | 199 |
| G48 | 194 | 199 |
| G49 | 195 | 199 |
| G50 | 196 | 199 |

In some embodiments, the anti-CD122 antibody comprises a series of CDR sequences. In some embodiments, the series of CDR sequences comprises an HCDR1 sequence, an HCDR2 sequence, an HCDR3 sequence, an LCDR1 sequence, an LCDR2 sequence, and an LCDR3 sequence. The series of CDR sequences for each anti-CD122 antibody described herein is listed in Table 20A. The names for the series of CDR sequences are E41-E56.

TABLE 20A
Series of CDR sequences for anti-CD122 antibodies

| Name | HCDR1 | HCDR2 | HCDR3 | LCDR1 | LCDR2 | LCDR3 |
|---|---|---|---|---|---|---|
| E41 | SEQ ID | SEQ ID | SEQ ID | SEQ ID | SEQ ID | SEQ ID |
| | NO: 235 | NO: 242 | NO: 245 | NO: 247 | NO: 250 | NO: 62 |
| E42 | SEQ ID | SEQ ID | SEQ ID | SEQ ID | SEQ ID | SEQ ID |
| | NO: 236 | NO: 239 | NO: 245 | NO: 247 | NO: 250 | NO: 62 |
| E43 | SEQ ID | SEQ ID | SEQ ID | SEQ ID | SEQ ID | SEQ ID |
| | NO: 233 | NO: 240 | NO: 245 | NO: 247 | NO: 250 | NO: 62 |
| E44 | SEQ ID | SEQ ID | SEQ ID | SEQ ID | SEQ ID | SEQ ID |
| | NO: 237 | NO: 243 | NO: 246 | NO: 248 | NO: 251 | NO: 253 |
| E45 | SEQ ID | SEQ ID | SEQ ID | SEQ ID | SEQ ID | SEQ ID |
| | NO: 235 | NO: 242 | NO: 245 | NO: 249 | NO: 252 | NO: 62 |
| E46 | SEQ ID | SEQ ID | SEQ ID | SEQ ID | SEQ ID | SEQ ID |
| | NO: 236 | NO: 239 | NO: 245 | NO: 249 | NO: 252 | NO: 62 |
| E47 | SEQ ID | SEQ ID | SEQ ID | SEQ ID | SEQ ID | SEQ ID |
| | NO: 233 | NO: 240 | NO: 245 | NO: 249 | NO: 252 | NO: 62 |
| E48 | SEQ ID | SEQ ID | SEQ ID | SEQ ID | SEQ ID | SEQ ID |
| | NO: 237 | NO: 243 | NO: 246 | NO: 248 | NO: 251 | NO: 253 |
| E49 | SEQ ID | SEQ ID | SEQ ID | SEQ ID | SEQ ID | SEQ ID |
| | NO: 231 | NO: 244 | NO: 245 | NO: 247 | NO: 250 | NO: 62 |
| E50 | SEQ ID | SEQ ID | SEQ ID | SEQ ID | SEQ ID | SEQ ID |
| | NO: 232 | NO: 239 | NO: 245 | NO: 247 | NO: 250 | NO: 62 |
| E51 | SEQ ID | SEQ ID | SEQ ID | SEQ ID | SEQ ID | SEQ ID |
| | NO: 233 | NO: 240 | NO: 245 | NO: 247 | NO: 250 | NO: 62 |
| E52 | SEQ ID | SEQ ID | SEQ ID | SEQ ID | SEQ ID | SEQ ID |
| | NO: 234 | NO: 243 | NO: 246 | NO: 248 | NO: 251 | NO: 253 |
| E53 | SEQ ID | SEQ ID | SEQ ID | SEQ ID | SEQ ID | SEQ ID |
| | NO: 231 | NO: 244 | NO: 245 | NO: 249 | NO: 252 | NO: 62 |
| E54 | SEQ ID | SEQ ID | SEQ ID | SEQ ID | SEQ ID | SEQ ID |
| | NO: 232 | NO: 239 | NO: 245 | NO: 249 | NO: 252 | NO: 62 |
| E55 | SEQ ID | SEQ ID | SEQ ID | SEQ ID | SEQ ID | SEQ ID |
| | NO: 233 | NO: 240 | NO: 245 | NO: 249 | NO: 252 | NO: 62 |
| E56 | SEQ ID | SEQ ID | SEQ ID | SEQ ID | SEQ ID | SEQ ID |
| | NO: 234 | NO: 243 | NO: 246 | NO: 248 | NO: 251 | NO: 253 |

In some embodiments, the anti-CD122 antibody CDR sequences can be defined by an antibody numbering scheme. In embodiments, the antibody numbering scheme is Kabat. In embodiments, the antibody numbering scheme is IMGT. In embodiments, the antibody numbering scheme is AbM. In embodiments, the antibody numbering scheme is Chothia. In embodiments, the antibody numbering scheme is Contact. In some embodiments, CDR sequences of anti-CD122 antibodies are numbering according to Table 20B.

TABLE 20B
CDR sequences for anti-CD122 antibody heavy chain variable domains and
light chain variable domains contained within a heavy chain or a light chain

| SEQ ID | | | | | | | |
|---|---|---|---|---|---|---|---|
| NO. of | | | | | | | |
| heavy | | | | | | | |
| chain | | SEQ | | SEQ | | SEQ | |
| from | Numbering | ID | HCDR1 | ID | | ID | HCDR3 |
| Table 6 | Scheme | NO: | Sequence | NO: | HCDR2 Sequence | NO: | Sequence |
| 94 or | Kabat | 231 | SHWIT | 238 | DIYPGSGNTNYNEK | 245 | ERGGFDY |
| 98 | | | | | FKS | | |
| | IMGT | 7 | GYTFTSHW | 18 | IYPGSGNT | 30 | ARERGGFD |
| | | | | | | | Y |
| | AbM | 232 | GYTFTSHWIT | 239 | DIYPGSGNTN | 245 | ERGGFDY |
| | Chothia | 233 | GYTFTSH | 240 | YPGSGN | 245 | ERGGFDY |
| | Contact | 234 | TSHWIT | 241 | WIGDIYPGSGNTN | 246 | ARERGGFD |
| 95 or | Kabat | 235 | SHWIS | 242 | DIYPGSGNTNYNEK | 245 | ERGGFDY |
| 99 | | | | | LQG | | |
| | IMGT | 7 | GYTFTSHW | 18 | IYPGSGNT | 30 | ARERGGFD |
| | | | | | | | Y |
| | AbM | 236 | GYTFTSHWIS | 239 | DIYPGSGNTN | 245 | ERGGFDY |
| | Chothia | 233 | GYTFTSH | 240 | YPGSGN | 245 | ERGGFDY |
| | Contact | 237 | TSHWIS | 243 | WMGDIYPGSGNTN | 246 | ARERGGFD |
| 96 or | Kabat | 231 | SHWIT | 244 | DIYPGSGNTNYNEK | 245 | ERGGFDY |
| 97 or | | | | | FQG | | |
| 100 or | IMGT | 7 | GYTFTSHW | 18 | IYPGSGNT | 30 | ARERGGFD |
| 101 | | | | | | | Y |
| | AbM | 232 | GYTFTSHWIT | 239 | DIYPGSGNTN | 245 | ERGGFDY |
| | Chothia | 233 | GYTFTSH | 240 | YPGSGN | 245 | ERGGFDY |
| | Contact | 234 | TSHWIT | 243 | WMGDIYPGSGNTN | 246 | ARERGGFD |
| SEQ ID | | | | | | | |
| NO. of | | | | | | | |
| light | | | | | | | |
| chain | | SEQ | | SEQ | | SEQ | |
| from | Numbering | ID | LCDR1 | ID | | ID | LCDR3 |
| Table 7 | Scheme | NO: | Sequence | NO: | LCDR2 Sequence | NO: | Sequence |
| 102 or | Kabat | 247 | RASQDISNFL | 250 | YTSGLHS | 62 | QQDNNHPY |
| 103 or | | | N | | | | T |
| 105 | IMGT | 43 | QDISNF | | YTS | 62 | QQDNNHPY |
| | | | | | | | T |
| | AbM | 247 | RASQDISNFL | 250 | YTSGLHS | 62 | QQDNNHPY |
| | | | N | | | | T |
| | Chothia | 247 | RASQDISNFL | 250 | YTSGLHS | 62 | QQDNNHPY |
| | | | N | | | | T |
| | Contact | 248 | SNFLNWY | 251 | LLIYYTSGLH | 253 | QQDNNHPY |
| 104 | Kabat | 249 | QASQDISNFL | 252 | YTSGLHT | 62 | QQDNNHPY |
| | | | N | | | | T |
| | IMGT | 43 | QDISNF | | YTS | 62 | QQDNNHPY |
| | | | | | | | T |
| | AbM | 249 | QASQDISNFL | 252 | YTSGLHT | 62 | QQDNNHPY |
| | | | N | | | | T |
| | Chothia | 249 | QASQDISNFL | 252 | YTSGLHT | 62 | QQDNNHPY |
| | | | N | | | | T |
| | Contact | 248 | SNFLNWY | 251 | LLIYYTSGLH | 253 | QQDNNHPY |

### Production and Manufacture of Antibodies or Antigen-Binding Fragments Thereof

In some embodiments, polypeptides described herein (e.g., antibodies or antigen-binding fragments thereof) are produced using any method known in the art to be useful for the synthesis of polypeptides (e.g., antibodies), in particular, by chemical synthesis or by recombinant expression, and are preferably produced by use of a recombinant expression technique.

In some instances, an antibody or antigen-binding fragment thereof is expressed recombinantly, and the nucleic acid encoding the antibody or its antigen-binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 5′ and 3′ ends of the sequence or by cloning using an oligonucleotide specific for the particular nucleic acid sequence.

In some instances, an antibody or its binding is optionally made by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937.

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, a nucleic acid sequence encodes the antibodies disclosed herein. In some embodiments, the polynucleotide sequence encoding the antibodies is operatively coupled to a eukaryotic regulatory sequence. In some embodiments, a cell comprises the nucleic acid sequence. In some embodiments, a cell comprises a nucleic acid encoding the antibodies disclosed herein. In some embodiments, the cell comprises a prokaryotic cell. In some embodiments, the prokaryotic cell is an Escherichia coli cell. In some embodiments, the cell comprises a eukaryotic cell. In some embodiments, the eukaryotic cell is a Chinese Hamster Ovary (CHO) cell, a HEK293 cell, a BHK cell, an NS0 murine myeloma cell, or a PER.C6® human cell. In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue, specific promoter. Standard cell lines and methods for the production of antibodies from a large-scale cell culture are known in the art. See e.g., Li et al., “Cell culture processes for monoclonal antibody production.” Mabs. 2010 September-October; 2(5): 466-477.

In certain aspects, described herein is a method of making antibodies comprising culturing a cell comprising a nucleic acid encoding a antibodies under conditions in vitro sufficient to allow production and secretion of the antibodies. In some embodiments, antibodies are harvested from the cell culture medium. The harvesting can further comprise one or more purification steps to remove live cells, cellular debris, non-antibody proteins or polypeptides, undesired salts, buffers, and medium components. In certain aspects, the additional purification step(s) include centrifugation, ultracentrifugation, protein A, protein G, protein A/G, or protein L purification, and/or ion exchange chromatography.

Combination Therapy for Treating GvHD (aGvHD and cGvHD)

Described herein are methods of prophylaxis for aGvHD and for cGvHD. Also described here are methods for treatment for aGvHD and for cGvHD. In one aspect, a method of prophylaxis for aGvHD comprises administering an effective amount of an anti-CD122 antibody combined with a JAK inhibitor. In some embodiments, the administering inhibits IL2 and/or IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, an anti-CD122 antibody described herein is administered as part of a combination therapy to the subject.

In one aspect, a method of prophylaxis for cGvHD comprises administering an effective amount of an anti-CD122 antibody. In some embodiments, the method further comprises administering a JAK inhibitor. In some embodiments, the administering inhibits IL2 and/or IL15 signaling. In some embodiments, the inhibition of IL2 and/or IL15 signaling occurs in cell types expressing intermediate affinity IL2R or IL15R composed of beta and gamma receptor subunits. In some embodiments, the administering an effective amount of an anti-CD122 antibody is combined with administering a JAK inhibitor. In some embodiments, an anti-CD122 antibody described herein is administered as part of a combination therapy to the subject.

In one aspect, a method for treatment of aGvHD comprises administering a therapeutically effective amount of an anti-CD122 antibody combined with a JAK inhibitor. In some embodiments, the administering inhibits IL2 and/or IL15 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor. In some embodiments, an anti-CD122 antibody described herein is administered as part of a combination therapy to the subject.

In one aspect, a method for treatment of cGvHD comprises administering a therapeutically effective amount of an anti-CD122 antibody. In some embodiments, the method further comprises administering a JAK inhibitor. In some embodiments, the administering inhibits IL2 and/or IL15 signaling. In some embodiments, the inhibition of IL2 and/or IL15 signaling occurs in cell types expressing intermediate affinity IL2R or IL15R composed of beta and gamma receptor subunits. In some embodiments, the administering a therapeutically effective amount of an anti-CD122 antibody is combined with administering a JAK inhibitor. In some embodiments, an anti-CD122 antibody described herein is administered as part of a combination therapy to the subject.

### JAK Inhibitors

In some instances, JAK inhibitors include JAK1 inhibitor, JAK2 inhibitor, and/or JAK3 inhibitor. In some instances, JAK inhibitors include JAK1/3 inhibitor, JAK1/2 inhibitor, or JAK1/2/3 inhibitor. In some instances, JAK inhibitor is a small molecule inhibitor, an antibody or antigen-fragment thereof, or a peptide inhibitor. In some embodiments, the JAK inhibitor administered to the subject is selected from abrocitinib, baricitinib, delgocitinib, fedratinib, filgotinib, oclacitinib, pacritinib, peficitinib, ruxolitinib, tofacitinib, itacitinib and upadacitinib. In some embodiments, the JAK inhibitor administered to the subject comprises ruxolitinib. Ruxolitinib can effectively target JAK1 and JAK2 for inhibition of JAK-mediated signaling. In some embodiments, the ruxolitinib is administered orally.

### Treatment Effects

In some embodiments, the methods of prevention or treatment of aGvHD achieve an effect in the subject. In some embodiments, an effective amount of an anti-CD122 antibody, or antigen-binding fragment thereof, is administered to the subject. In some embodiments, an effective amount of an anti-CD122 antibody, or antigen-binding fragment thereof, is administered to the subject in need of prevention or treatment of aGvHD. In some embodiments, the anti-CD122 antibody, or antigen-binding fragment thereof, is administered daily. In some embodiments, administering an effective amount of the anti-CD122 antibody, or antigen-binding fragment thereof, delays an onset of one or more symptoms of aGvHD in the subject. In some embodiments, administering an effective amount of the anti-CD122 antibody, or antigen-binding fragment thereof, alleviates one or more symptoms of aGvHD in the subject. In subjects affected by aGvHD, the one or more symptoms of aGvHD may develop within the first 100 days following alloHSCT. In subjects affected by aGvHD, the one or more symptoms of aGvHD may develop after the first 100 days following alloHSCT. In some embodiments, administering an effective amount of the anti-CD122 antibody, or antigen-binding fragment thereof, changes one or more molecular markers of aGvHD (e.g., expression, distribution, etc.).

In some embodiments, an effective amount of an anti-CD122 antibody, or antigen-binding fragment thereof, is administered to the subject in combination with administering an effective amount of a JAK inhibitor. In some embodiments, an effective amount of an anti-CD122 antibody, or antigen-binding fragment thereof, is administered to the subject in need of prevention or treatment of aGvHD in combination with administering an effective amount of the JAK inhibitor. In some embodiments, the anti-CD122 antibody, or antigen-binding fragment thereof, and the JAK inhibitor are administered sequentially, separately, or are co-administered. In some embodiments, administering an effective amount of the anti-CD122 antibody, or antigen-binding fragment thereof, in combination with administering an effective amount of the JAK inhibitor delays an onset of one or more symptoms of aGvHD in the subject. In some embodiments, administering an effective amount of the anti-CD122 antibody, or antigen-binding fragment thereof, in combination with administering an effective amount of the JAK inhibitor alleviates one or more symptoms of aGvHD in the subject. In subjects affected by aGvHD, the one or more symptoms of aGvHD may develop within the first 100 days following alloHSCT. In subjects affected by aGvHD, the one or more symptoms of aGvHD may develop after the first 100 days following alloHSCT. In some embodiments, administering an effective amount of the anti-CD122 antibody, or antigen-binding fragment thereof, in combination with JAK inhibitor changes one or more molecular markers of aGvHD (e.g., expression, distribution, etc.).

In some embodiments, the symptoms of aGvHD to be treated comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss. In some embodiments, the rash on the subject can erupt on the skin of the palms or on the soles of the feet. In some embodiments, the rash on the subject may involve the skin of the trunk or the skin the subject's extremities. In some embodiments, administering an effective amount of the anti-CD122 antibody, or antigen-binding fragment thereof, increases the survival rate of the subject. In some embodiments, administering an effective amount of the anti-CD122 antibody, or antigen-binding fragment thereof, decreases the risk of aGvHD-symptom relapse. In some embodiments, administering an effective amount of the anti-CD122 antibody, or antigen-binding fragment thereof, decreases the risk that the subject will develop cGvHD. In some embodiments, administering an effective amount of the anti-CD122 antibody, or antigen-binding fragment thereof, in combination with administering an effective amount of the JAK inhibitor increases the survival rate of the subject. In some embodiments, administering an effective amount of the anti-CD122 antibody, or antigen-binding fragment thereof, in combination with administering an effective amount of the JAK inhibitor decreases the risk of aGvHD-symptom relapse. In some embodiments, administering an effective amount of the anti-CD122 antibody, or antigen-binding fragment thereof, in combination with administering an effective amount of the JAK inhibitor decreases the risk that the subject will develop cGvHD.

In some instances, the methods of prevention or treatment of cGvHD achieve an effect in the subject. In some embodiments, an effective amount of an anti-CD122 antibody, or antigen-binding fragment thereof, is administered to the subject. In some embodiments, an effective amount of an anti-CD122 antibody, or antigen-binding fragment thereof, is administered to the subject in need of prevention or treatment of cGvHD. In some embodiments, administering an effective amount of the anti-CD122 antibody, or antigen-binding fragment thereof, delays an onset of one or more symptoms of cGvHD in the subject. In some embodiments, administering an effective amount of the anti-CD122 antibody, or antigen-binding fragment thereof, alleviates or eliminates one or more symptoms (e.g., frequencies of the symptoms, intensity of the symptoms, etc.) of cGvHD in the subject. In some embodiments, administering an effective amount of the anti-CD122 antibody, or antigen-binding fragment thereof, changes one or more molecular markers of cGvHD (e.g., expression, distribution, etc.

In some embodiments, an effective amount of an anti-CD122 antibody, or antigen-binding fragment thereof, is administered to the subject in need of prevention or treatment of cGvHD in combination with administering an effective amount of a JAK inhibitor. In some embodiments, the anti-CD122 antibody, or antigen-binding fragment thereof, and the JAK inhibitor are administered sequentially, separately, or are co-administered. In some embodiments, administering an effective amount of the anti-CD122 antibody, or antigen-binding fragment thereof, in combination with administering an effective amount of the JAK inhibitor delays an onset of one or more symptoms of cGvHD in the subject. In some embodiments, administering an effective amount of the anti-CD122 antibody, or antigen-binding fragment thereof, in combination with administering an effective amount of the JAK inhibitor alleviates one or more symptoms of cGvHD in the subject.

In some embodiments, the symptoms of cGvHD to be treated comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, and changes in hair texture. In some embodiments, the painful mouth ulcers extend down the throat of the subject. In some embodiments, administering an effective amount of the anti-CD122 antibody, or antigen-binding fragment thereof, increases the survival rate of the subject. In some embodiments, administering an effective amount of the anti-CD122 antibody, or antigen-binding fragment thereof, in combination with administering an effective amount of the JAK inhibitor increases the survival rate of the subject. In some embodiments, administering an effective amount of the anti-CD122 antibody, or antigen-binding fragment thereof, decreases the risk of cGvHD-symptom relapse. In some embodiments, administering an effective amount of the anti-CD122 antibody, or antigen-binding fragment thereof, in combination with administering an effective amount of the JAK inhibitor decreases the risk of cGvHD-symptom relapse.

### Pharmaceutical Compositions

Provided here a pharmaceutical compositions comprising an antibody or an antibody fragment and at least one pharmaceutically acceptable carrier. In some embodiments, the antibody or antibody fragment binds to CD122. In some embodiments, the antibody or antibody fragment is an anti-CD122 antibody or anti-CD122 antibody fragment. In some embodiments, the pharmaceutical compositions comprises an anti-CD122 antibody described herein. In some embodiments, the antibody or antibody fragment binds to human CD122. In some embodiments, the antibody or an antibody fragment thereof and at least one pharmaceutically acceptable carrier are formulated into a pharmaceutical formulation. In some embodiments, the pharmaceutical formulation is chosen based on a preferred route of administration of the antibody or antibody fragment to a subject.

In some embodiments, the pharmaceutical formulations include, but are not limited to, aqueous liquid dispersions, self-emulsifying dispersions, solid solutions, liposomal dispersions, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanorods, nanoropes, and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle. In some instances, nanoparticles comprise nanospheres or nanocapsules.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginate, pectin, carrageenan, fucoidan, agaropectin, porphyran, karaya gum, xanthan gum, hyaluronic acids, glucosamine, galactosamine, chitosan, polyglutamic acid, polyaspartic acid, lysozyme, cytochrome C, trypsinogen, chymotrypsinogen, α-chymotrypsin, polylysine, polyarginine, histone, protamine, ovalbumin, or dextrin or cyclodextrin.

In some embodiments, the pharmaceutical formulations described herein are administered to a subject by multiple administration routes, including but not limited to, parenteral (e.g., intravenous, subcutaneous, intramuscular), oral, intranasal, buccal, vaginal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration.

### Pharmaceutically Acceptable Excipients, Carriers, and Diluents

Compositions comprising the antibodies of the current disclosure are included in a pharmaceutical composition comprising one or more pharmaceutically acceptable excipients, carriers, and diluents. In some embodiments, the antibodies of the current disclosure are administered suspended in a sterile solution. In some embodiments, the antibodies of the current disclosure are administered suspended in an isotonic solution. In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. In certain aspects, the solution comprises about 0.9% NaCl. In certain aspects, the solution comprises about 5.0% dextrose. In certain aspects, the solution further comprises one or more of buffers, for example, acetate, citrate, histidine, succinate, phosphate, bicarbonate and Tris(hydroxymethyl)aminomethane; surfactants, for example, polysorbate 80 (Tween 80), polysorbate 20 (Tween 20);
polyol/disaccharide/polysaccharides, for example, glucose, dextrose, mannose, mannitol, sorbitol, sucrose, and dextran 40; amino acids, for example, glycine or arginine; antioxidants, for example, ascorbic acid, methionine; or chelating agents, for example, EDTA or EGTA.

In certain aspects, the antibodies of the current disclosure are shipped and/or stored lyophilized and can then be reconstituted before administration. In certain aspects, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than about 7.5. In certain aspects, the pH can be between 4.5 and 7.5, 4.5 and 7.0, 4.5 and 6.5, 4.5 and 6.0, or 5.5 or 5.0.

### Delivery of Anti-CD122 Antibody and JAK Inhibitors

In some aspects, an anti-CD122 antibody is delivered to the subject by a certain route of administration. In some embodiments, the anti-CD122 antibody is delivered systemically, locally, intradermally, subcutaneously, or topically. In some embodiments, the anti-CD122 antibody delivered systemically is administered by intravenous injection, by intraperitoneal injection, by subcutaneous injection, by enteral administration, or through inhalation. In some embodiments, the anti-CD122 antibody delivered systemically is administered by intravenous injection according to a treatment regimen specifying a frequency of administration. In some embodiments, the anti-CD122 antibody delivered by enteral administration is administered orally. In some embodiments, the anti-CD122 antibody delivered by enteral administration is administered vaginally or rectally. In some embodiments, the anti-CD122 antibody delivered locally is administered topically. In some embodiments, the anti-CD122 antibody delivered locally is administered intradermally.

In some aspects, a JAK inhibitor is delivered to the subject by a certain route of administration. In some embodiments, the JAK inhibitor is delivered systemically, locally, intradermally, subcutaneously, or topically. In some embodiments, the JAK inhibitor delivered systemically is administered by intravenous injection, by subcutaneous injection, by enteral administration, or through inhalation. In some embodiments, the JAK inhibitor delivered systemically is administered by intravenous injection according to a treatment regimen specifying a frequency of administration. In some embodiments, the JAK inhibitor delivered by enteral administration is administered orally. In some embodiments, the JAK inhibitor delivered systemically by oral administration is administered according to a treatment regimen specifying a frequency of administration. In some embodiments, the JAK inhibitor delivered by enteral administration is administered vaginally or rectally. In some embodiments, the JAK inhibitor delivered locally is administered topically. In some embodiments, the JAK inhibitor delivered locally is administered subcutaneously. In some embodiments, the JAK inhibitor delivered locally is administered intradermally.

In some aspects, an anti-CD122 antibody and a JAK inhibitor are administered as a combination therapy. In some embodiments, the anti-CD122 antibody and the JAK inhibitor are co-administered. In some instances, the anti-CD122 antibody and the JAK inhibitor are formulated in a single pharmaceutical composition and administered concurrently. In some instances, the anti-CD122 antibody and the JAK inhibitor are formulated in two separate pharmaceutical compositions and the two pharmaceutical compositions are administered concurrently. In some embodiments, the anti-CD122 antibody and the JAK inhibitor are co-administered substantially simultaneously. In some embodiments, the anti-CD122 antibody and the JAK inhibitor are administered sequentially. In some embodiments, the anti-CD122 antibody is administered first and the JAK inhibitor is administered second. In some embodiments, the JAK inhibitor is administered first and the anti-CD122 antibody is administered second. In some embodiments, the anti-CD122 antibody and the JAK inhibitor are administered separately. In some embodiments, the anti-CD122 antibody is administered by intravenous injection and the JAK inhibitor is administered orally. In some embodiments, the anti-CD122 antibody is administered by intraperitoneal injection and the JAK inhibitor is administered orally. In some embodiments, the anti-CD122 antibody is formulated together with a pharmaceutically acceptable excipient according to a chosen route of administration. In some embodiments, the JAK inhibitor is formulated together with a pharmaceutically acceptable excipient according to a chosen route of administration.

In some instances, the anti-CD122 antibody and the JAK inhibitor are administered to the subject via the same route of administration (e.g., both systemically, both locally, both intradermally, both cutaneously, both via i.v. injection, etc.). In some instances, the anti-CD122 antibody and the JAK inhibitor are administered to the subject via two separate routes of administration (e.g., anti-CD122 antibody via i.v. injection and the JAK inhibitor or subcutaneously, anti-CD122 antibody orally and the JAK inhibitor intradermally, etc.).

In some embodiments, an immunosuppressive agent is administered to the subject. In some embodiments, two or more immunosuppressive agents are administered to the subject. In some embodiments, an immunosuppressive agent is administered to the subject for prophylaxis of aGvHD. In some embodiments, an immunosuppressive agent is administered to the subject for prophylaxis of cGvHD. In some embodiments, an immunosuppressive agent is administered to the subject for treating one or more symptoms of aGvHD. In some embodiments, an immunosuppressive agent is administered to the subject for treating one or more symptoms of cGvHD. In some embodiments, the immunosuppressive agent is administered separately from the anti-CD122 antibody. In some embodiments, the immunosuppressive agent and the anti-CD122 antibody are administered sequentially. In some embodiments, the immunosuppressive agent is cyclosporine. In some embodiments, the immunosuppressive agent is tacrolimus. In some embodiments, the immunosuppressive agent is antithymocyte globulin. In some embodiments, the immunosuppressive agent is alemtuzumab. In some embodiments, the immunosuppressive agent is belumosudil. In some embodiments, belumosudil is administered for prophylaxis of cGvHD or for treating cGvHD. In some embodiments, the immunosuppressive agent is ibrutinib. In some embodiments, ibrutinib is administered for prophylaxis of cGvHD or for treating cGvHD. In some embodiments, the immunosuppressive agent is cyclophosphamide. In some embodiments, the immunosuppressive agent is methotrexate. In some embodiments, the immunosuppressive agent is mycophenolate mofetil. In some embodiments, the immunosuppressive agent is sirolimus.

### Treatment Regimens

Described herein are methods of administration of an anti-CD122 antibody described herein that comprise a treatment regimen. In some embodiments, the method comprises a treatment regimen delivering a pharmaceutical formulation of the anti-CD122 antibody described herein according to the needs of a subject. In some embodiments, the treatment regimen comprises a single administration. In some embodiments, the treatment regimen comprises at least one administration. In some embodiments, the treatment regimen comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, S′6, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more administrations. In some embodiments, the effective amount contained within one administration is an effective amount for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 60, 61, 62, or 90 days. In some embodiments, one administration comprises an effective amount for at least 2 days, at least 3 days, at least 4 days, at least 5 days, at least 1 week, at least 2 weeks, at least 4 weeks, at least 2 months, or at least 6 months. In some embodiments, the method further comprises a treatment cycle. In some embodiments, the treatment cycle comprises 1, 2, 3, 4, 5, 6, 7, 8, 9, 10, 11, 12, 13, 14, 15, 16, 17, 18, 19, 20 or more administrations of the pharmaceutical composition. In some embodiments, the treatment regimen comprises a plurality of treatment cycles. In some embodiments, the plurality of treatment cycles is 1, 2, 3, 4, 5, 6, 7, 8, 9, or 10 more treatment cycles. In some embodiments, a treatment cycle comprises a length of time. In some embodiments, the length of time comprises 1, 2, 3, 4, 5, 6, 7, 8, 9, 10, 11, 12, 13, 14, 15, 16, 17, 18, 19, 20, 21, 22, 23, 24, 30, or 36 months. In some embodiments, a treatment is administered at a selected interval such as once every 1 day, 2 days, 3 days, 4 days, 5 days, 6 days, 7 days, 8 days, 9 days, 10 days 11 days, 12 days, 13 days, 14 days, 15 days, 16 days, 17 days, 18 days, 19 days, 20 days or 21 days. In some embodiments, a treatment is administered at a selected interval such as once every 1 week, 2 weeks, 3 weeks, 4 weeks, 5 weeks, 6 weeks, 7 weeks, 8 weeks, 9 weeks, 10 weeks, 11 weeks, or 12 weeks.

In some embodiments, the treatment regimen comprises combination therapy of administering a pharmaceutical composition comprising an anti-CD122 antibody described herein in combination with a JAK inhibitor. In some embodiments, the pharmaceutical composition comprising the anti-CD122 antibody and the JAK inhibitor are administered separately as part of the treatment regimen. In some embodiments, the pharmaceutical composition comprising the anti-CD122 antibody and the JAK inhibitor are administered simultaneously as part of the treatment regimen. In some embodiments, the pharmaceutical composition comprising the anti-CD122 antibody is administered systemically. In some embodiments, the systemic administration comprises intravenous injection. In some embodiments, the pharmaceutical composition comprising the anti-CD122 antibody is administered locally. In some embodiments, local administration comprises topical application or subcutaneous injection. In some embodiments, the JAK inhibitor is administered systemically. In some embodiments, the systemic administration of the JAK inhibitor comprises oral administration of the JAK inhibitor. In some embodiments, the JAK inhibitor is administered locally. In some embodiments, the dosage of the pharmaceutical composition comprising the anti-CD122 antibody is a fixed dosage. In some embodiments, the dosage of the pharmaceutical composition comprising the anti-CD122 antibody is a variable dosage based on the body weight of the subject and/or the severity of the symptoms in the subject and/or the extent of response in the subject to a previous treatment. In some embodiments, the dosage of the JAK inhibitor is a fixed dosage. In some embodiments, the dosage of the JAK inhibitor is a variable dosage based on the body weight of the subject and/or the severity of the symptoms in the subject and/or the extent of response in the subject to a previous treatment.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “effective amount” or “therapeutically effective amount” refers to an amount of an antibody effective to prevent, in methods of prophylaxis, or to treat, in methods of treatment a disease or disorder in the subject or mammal. In some cases, a therapeutically effective amount of the antibody reduces the severity of symptoms of the disease or disorder. In some instances, the disease or disorder comprises aGvHD. In some instances, the disease or disorder comprises cGvHD. In some instances, the aGvHD or the cGvHD are severe or medically refractory forms the aGvHD or cGvHD. In some instances, the severity of aGvHD has been assigned a grade of severity from grade 1 to grade 4; (grade 1: mild aGvHD, grade 2: moderate aGvHD, grade 3: severe aGvHD, grade 4: very severe aGvHD). In some instances, signs and symptoms of chronic GVHD vary between subjects and in the same subject over time. In some instances, the subject is scored for involvement of individual organs on a 0 to 3 scale, from no involvement/no symptoms to severe functional compromise. In some instances, therapeutically effective amount can be the same or different from a prophylactically effective amount, which is an amount necessary to prevent onset of disease or disease symptoms. An effective amount can be administered in one or more administrations or dosages. A therapeutically effective amount of a therapeutic antibody (e.g., an effective dosage) depends on the antibody selected. The skilled artisan will understand that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the antibody described herein may include a single treatment or a series of treatments. Dosage, toxicity and therapeutic efficacy of the antibody can be determined by standard pharmaceutical procedures in cell cultures or experimental animals, e.g., for determining the ED50 (the dose therapeutically effective in 50% of the population). The data obtained from cell culture assays and animal studies can be used in formulating a range of dosage for use in humans. In some instances, the effective amount is a fixed dosage determined for the subject. In some instances, the effective amount is a variable dosage determined for the subject, based on a parameter such as body weight of the subject, severity of symptoms present in the subject, and/or response to one or more previous treatments in the subject.

The term “antibody” herein is used in the broadest sense and includes monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. An antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A monoclonal antibody can comprise a human IgG1 constant region or a human IgG4 constant region.

The terms “complementarity determining region,” and “CDR,” which are synonymous with “hypervariable region” or “HVR,” are known in the art and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain aspects, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

Among the provided antibodies are antigen-binding fragments thereof. An “antigen-binding fragment thereof” derived from an antibody can refer to a molecule other than an intact antibody that comprises a portion of an intact antibody that binds the antigen to which the intact antibody binds. Examples of antigen-binding fragments thereof derived from an antibody include, but are not limited to, Fv, Fab, Fab′, Fab′-SH, F(ab′)2; diabodies; linear antibodies; and single-chain antibody molecules (e.g. scFv or sFv). In particular aspects, the antibodies are single-chain antibody fragments comprising a variable heavy chain region and/or a variable light chain region, such as scFvs. Antigen-binding fragments thereof derived from an antibody can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. The strength, or affinity of immunological binding interactions of antibodies and/or antigen-binding fragments (hereof can be expressed in terms of the dissociation constant (K_{d}) of a specific interaction, wherein a smaller Kd represents a greater affinity for the antibody or antigen-binding fragment thereof to an antigen. Immunological binding properties of selected polypeptides described herein can be quantified using methods well known in the art One such method involves measuring the rates of antigen-binding site/antigen complex formation and dissociation, wherein those rates depend on the concentrations of the complex partners, the affinity of the interaction, and various geometric parameters that equally influence the rate in both directions. Thus, both the “on rate constant” (Kₒₙ) and the “off rate constant” (K_{off}) may be determined by calculation of the concentrations and the actual rates of association and dissociation.

A “binding moiety” refers to a portion of a molecule, peptide, polypeptide, antibody, or antibody fragment that mediates specific binding to a recited target or antigen or epitope. By way of example, the binding moiety of an antibody may comprise a heavy-chain/light-chain variable region pair or one or more complementarity determining regions (CDRs).

A “target” as referred to herein refers to the portion of a molecule that participates with a binding moiety of a molecule, peptide, polypeptide, antibody, or antibody fragment. A target can comprise an amino acid sequence and/or a carbohydrate, lipid, or other chemical entity. An “antigen” is a target comprising a portion that is able to be bound by an adaptive immune molecule such as an antibody or antibody fragment, B-cell receptor, or T-cell receptor.

An “epitope” as described herein refers to the one or more contact regions of an antibody. The contact region of an antibody consists of a discreet number of amino acids contacted by amino acid residues of the antibody (generally CDR residues) and adjacent residues contiguous with the contact residues. For example, the contact region may consist of a continuous stretch of a target protein that is between 5 to 20 amino acids, 5 to 15 amino acids, or 5 to 10 amino acids. An antibody may bind more than one contact region that are separated by 10, 20, 30, 40, 50, 75, or 100 amino acids or more as a result of protein folding. Epitopes may be determined using X-ray crystallography, hydrogen-deuterium exchange mass-spec, alanine spanning mutagenesis, competition with excess synthetic peptides as determined by immunoblot, ELISA, surface plasmon radiance, flow cytometry or any other suitable protein binding assay.

A “humanized” antibody is an antibody in which all or substantially all CDR amino acid residues are derived from non-human CDRs and all or substantially all FR amino acid residues are derived from human FRs. A humanized antibody optionally can include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

“ADCC” or “antibody dependent cell-mediated cytotoxicity” as used herein, refers to the cell-mediated reaction wherein nonspecific cytotoxic cells that express FcγRs recognize bound antibody on a target cell and subsequently cause lysis of the target cell. ADCC can be correlated with binding to FcγRIIIa wherein increased binding to FcγRIIIa leads to an increase in ADCC activity. “ADCP” or antibody dependent cell-mediated phagocytosis, as used herein, can refer to the cell-mediated reaction wherein nonspecific cytotoxic cells that express FcγRs recognize bound antibody on a target cell and subsequently cause phagocytosis of the target cell.

The terms “polypeptide” and “protein” are used interchangeably and refers to a polymer of amino acid residues, and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some embodiments, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence variants of the antibodies provided herein can be contemplated and conceived. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Antibody variants having one or more amino acid substitutions can be provided. Sites of interest for mutagenesis by substitution include the CDRs and FRs. Amino acid substitutions can be introduced into an antibody of interest and the products screened for a desired activity, e.g., retained/improved antigen binding, decreased immunogenicity, or improved ADCC or CDC.

The antibodies described herein can be encoded by a nucleic acid. A nucleic acid is a type of polynucleotide comprising two or more nucleotide bases. In certain aspects, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors, and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2(5): 466-477. In certain aspects, the cell is a Eukaryotic cell. In certain aspects, the Eukaryotic cell is a mammalian cell. In certain aspects, the mammalian cell is a cell line useful for producing antibodies is a Chines Hamster Ovary cell (CHO) cell, an NS0 murine myeloma cell, or a PER.C6® cell. In certain aspects, the nucleic acid encoding the antibody is integrated into a genomic locus of a cell useful for producing antibodies. In certain aspects, described herein is a method of making an antibody comprising culturing a cell comprising a nucleic acid encoding an antibody under conditions in vitro sufficient to allow production and secretion of said antibody.

The section headings used herein are for organizational purposes only and are not to be construed as limiting the subject matter described.

### EXAMPLES

The following examples are included for illustrative purposes only and are not intended to limit the scope of the invention.

### Example 1: Acute GvHD Study for Dose-Ranging Investigation of Antibody 1

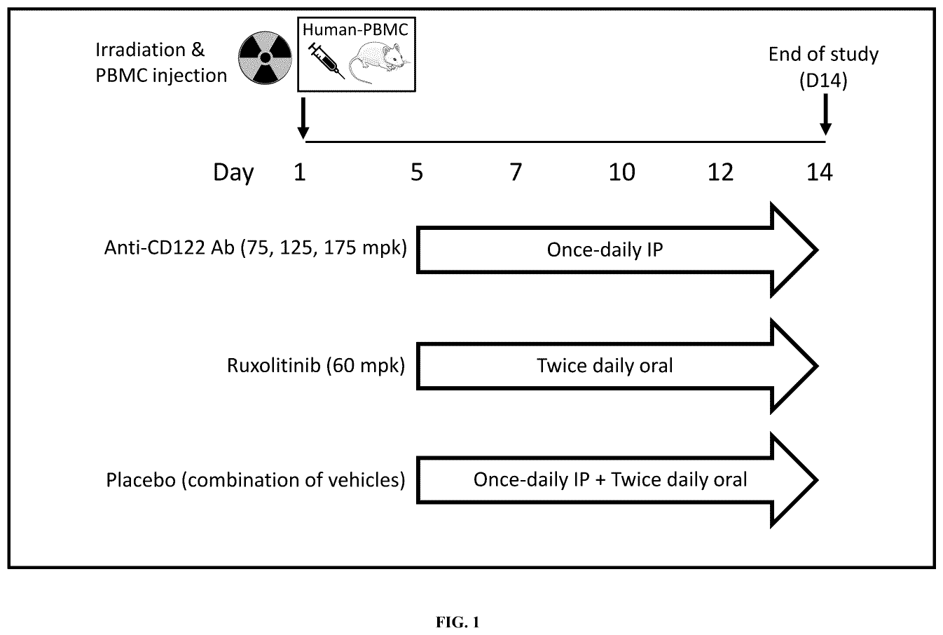

A xenograft GvHD mouse model was utilized to study human T and NK cell-mediated aGvHD and the therapeutic effects of anti-CD122 antibody treatment therein. Female NCG-hIL-15 mice were irradiated at a dosage of 100 cGy on Day 1 of the protocol and human peripheral blood mononuclear cells (PBMCs) were transplanted via injection at a dosage of 1×10⁷ cells/200 μL/mice via IV administration into the irradiated mice. PBMCs are immune cells that originate in the bone marrow and are secreted into the peripheral circulation forming critical components of the immune system. PBMCs are involved in humoral and cell-mediated immune function. As seen from the study design in FIG. 1, treatment with an anti-CD122 antibody was begun on Day 5 following transplant. Groups of 10 mice were used for each test category. A vehicle group (n=10) served as a negative control with no treatment administered (only a combination of vehicle treatments) following transplant. A positive control group (n=10) of mice treated with 60 mcg each of ruxolitinib twice daily by oral administration was included in the study design. Ruxolitinib-treated animals were treated via oral administration of ruxolitinib twice daily on D5, D6, D7, D8, D9, D10, D11, D12, D13, and D14. Three groups of Antibody 1 treated animals (n=10 for each group) were tested using a) 75 milligram per kilogram of body weight (mpk) of antibody, b) 125 mpk of antibody, and c) 175 mpk of antibody, respectively. Animals were treated via intraperitoneal (IP) injection for administration of Antibody 1 (anti-CD122 antibody as described herein) once daily on D5, D6, D7, D8, D9, D10, D11, D12, D13, and D14. The study concluded on Day 14.

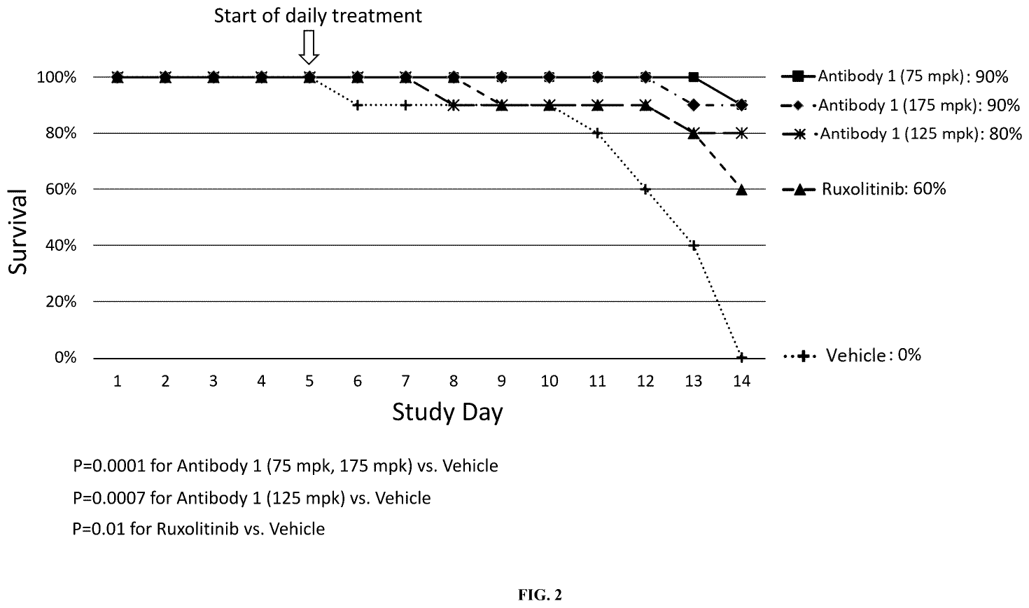

FIG. 2 demonstrates that an aggressive form of aGvHD was established with this protocol. Treating the animals with 75, 125, or 175 mpk of Antibody 1 resulted in a marked improvement in survival at study conclusion of Day 14. 75 mpk and 175 mpk treated animals had 90% survival at study conclusion (p=0.0001 vs. vehicle by Fisher's exact test). 125 mpk of Antibody 1 treatment had 80% survival at study conclusion (p=0.0007 vs. vehicle). FIG. 2 shows the results of ruxolitinib treatment on survival. Ruxolitinib-treated animals had 60% survival at study conclusion (p=0.01 vs. vehicle). Vehicle-treated animals had 0% survival by Day 14. These results demonstrate that anti-CD122 antibody therapy using an antibody described in this specification is an effective therapeutic strategy for aGvHD.

### Example 2: Acute GvHD Study Comparing Single and Combination Therapies

A xenograft GvHD mouse model was utilized to study human T and NK cell-mediated aGvHD and the therapeutic effects of anti-CD122 antibody treatment in single or combination therapy approaches. Female NCG-hIL-15 mice were irradiated at a dosage of 100 cGy on Day 1 of the protocol and human peripheral blood mononuclear cells (PBMCs) were transplanted via injection at a dosage of 1×10⁷ cells/200 μL/mice via IV administration into the irradiated mice according to the same xenograft GvHD mouse model design as in Example 1.

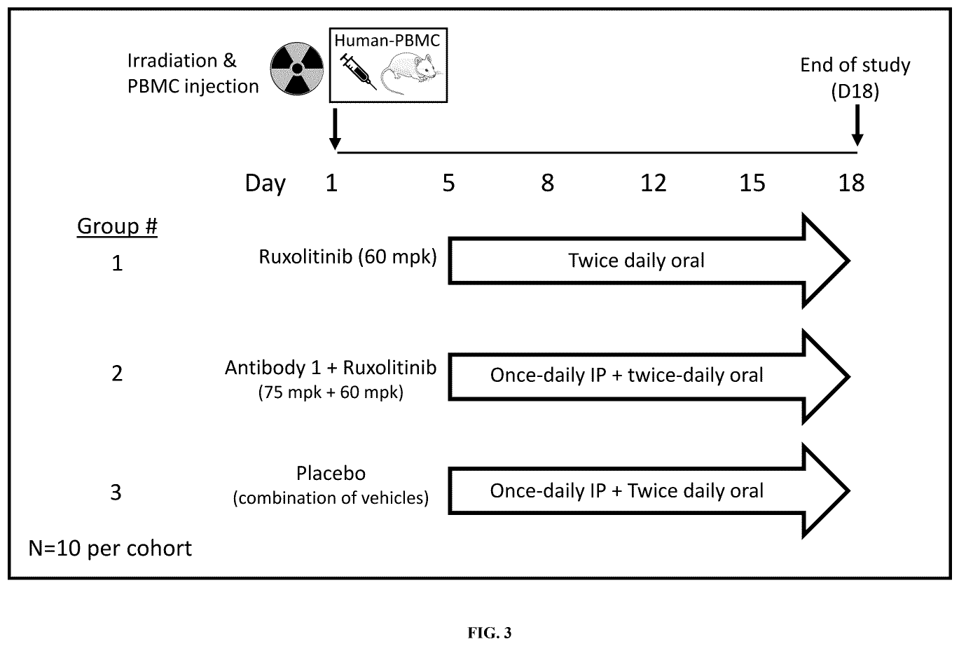

Treatment according to 3 different grouping of animals was begun on Day 5 following transplant, as shown in the study design of FIG. 3. Groups of 10 mice were used for each test category. A single treatment group, Group #1, (n=10) received ruxolitinib (at a dosage of 60 mpk) twice-daily by oral administration beginning on Day 5 and ending on Day 18. Days of treatment were D5, D6, D7, D8, D9, D10, D11, D12, D13, D14, D15, D16, D17, and D18. A combination therapy group, Group #2, (n=10) received ruxolitinib (at a dosage of 60 mpk) twice-daily by oral administration beginning on Day 5 and ending on Day 18. Days of treatment using ruxolitinib in Group #2 were D5, D6, D7, D8, D9, D10, D11, D12, D13, D14, D15, D16, D17, and D18. Group #2 animals separately received treatment with Antibody 1 (anti-CD122 antibody as described herein) at a dosage of 75 mpk once-daily by IP administration. Days of treatment using Antibody 1 in Group #2 were D5, D6, D7, D8, D9, D10, D11, D12, D13, D14, D15, D16, D17, and D18. A vehicle group, Group #3, (n=10) served as a negative control with vehicle-only administered following transplant received twice daily oral placebo (vehicle only) beginning on Day 5 and ending on Day 18. Days of oral placebo treatment were D5, D6, D7, D8, D9, D10, D11, D12, D13, D14, D15, D16, D17, and D18. This vehicle group also separately received once daily IP placebo (vehicle only) on D5, D6, D7, D8, D9, D10, D11, D12, D13, D14, D15, D16, D17, and D18.

Day 18 was the end of study day for all treatment groups. Animals from all treatment groups were assessed daily beginning on Day 1 and ending on Day 18 for survival following transplant. A comparison of survival rate between Group #1 (single treatment group with ruxolitinib monotherapy), Group #2 (combination therapy group with ruxolitinib and Antibody 1 combination treatment), and Group #3 (placebo group serving as negative control) was undertaken to evaluate treatment effects on subject survival rate daily throughout the study and to evaluate overall survival rate at the end of the study. At the end of the study, tissue was collected from all surviving animals for histological assessment of key target organs. These key target organs include liver, lung, and skin. A histological and phenotypic analysis of collected tissue is undertaken to determine the presence and extent of aGvHD symptoms at an organ level, a tissue level, a cellular level, and a molecular level. A comparison of histological findings between treatment groups is undertaken to determine which treatment groups yield the most favorable outcomes.

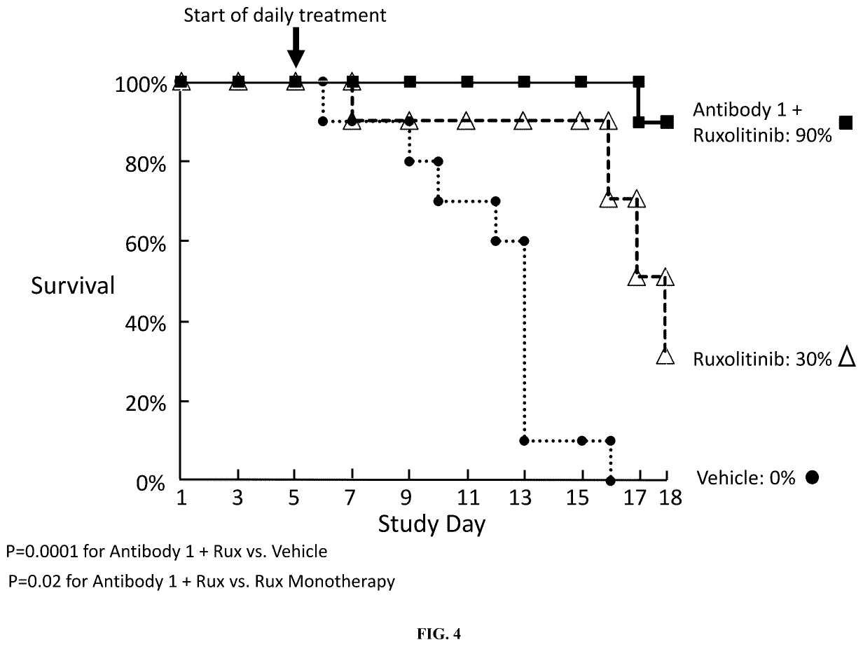

This study showed a therapeutic benefit for survival in this model of aGvHD for animals receiving combination therapy of ruxolitinib treatment and Antibody 1 treatment compared with either ruxolitinib single therapy or compared with placebo-treatment using xenotransplant from a specific donor of human PBMCs. As shown in FIG. 4, survival for treatment group #1, #2, and #3 was assessed daily and plotted to assess survival duration and compare by treatment group. At the end of the study, Group #2 (Antibody 1+ruxolitinib) animals had a 90% survival rate, Group #1 (ruxolitinib) animals had a 30% survival rate, and Group #3 (control) animals had a 0% survival rate. Statistical analysis using Fisher's exact test indicated that combination therapy of ruxolitinib treatment and Antibody 1 treatment yielded a statistically significant improved result in survival rate of subjects compared with ruxolitinib single therapy (p-value=0.02). Statistical analysis using Fisher's exact test indicated that combination therapy of ruxolitinib treatment and Antibody 1 treatment yielded a statistically significant improved result in survival rate of subjects compared with placebo-treatment (p-value=0.0001). Combination therapy for aGvHD comprising administering anti-CD122 antibody and ruxolitinib demonstrated a drastic improvement in survival rate compared with ruxolitinib monotherapy.

This result may indicate a synergistic effect of targeting the IL2, IL15, or IL2 and IL15 signaling pathways at different levels of the signal transduction cascade (e.g., targeting IL2 and/or IL15 signal reception and/or initiation of transduction with treatment using anti-CD122 antibody and targeting downstream signal transduction through modulating protein phosphorylation by inhibiting Janus kinases to ultimately yield a more robust effect of modulating IL2 and/or IL15 signal transduction responses in cells, tissues, and organs subjected to the immunological and pathology effects of aGvHD). This result indicates that combination treatment of anti-CD122 antibody therapy combined with JAK inhibitor therapy can improve survival outcome in an aGvHD model compared with JAK inhibitor as a monotherapy.

Table 21 lists survival rates of different groups on Day 18 tested in this example.

TABLE 21
Survival rates of different groups on Day 18

| | Average | Median | P values | P values |
|---|---|---|---|---|
| | survival time | survival | (vs. | (vs. |
| Group | (days) | (days) | Group #3) | Group #1) |
| Group #1 (ruxolitinib | 16.3 ± 3.4 | 17.5 | 0.002* | — |
| monotherapy) | | | | |
| Group #2 | 17.9 ± 0.3 | >18 | <0.001*** | 0.007** |
| (combination | | | | |
| therapy: Antibody | | | | |
| 1 + ruxolitinib) | | | | |
| Group #3 (Placebo - | 11.8 ± 3.1 | 13 | — | / |
| vehicle control) | | | | |

Data shown as Mean ± SD. Statistics were performed using Log-rank test compared to Group #3 or Group #1, *: P ≤ 0.05, **: P ≤ 0.01, ***: P ≤ 0.001.

### Example 3: Chronic GvHD Study to Investigate Anti-CD122 Antibody Monotherapy or in Combination with a JAK Inhibitor

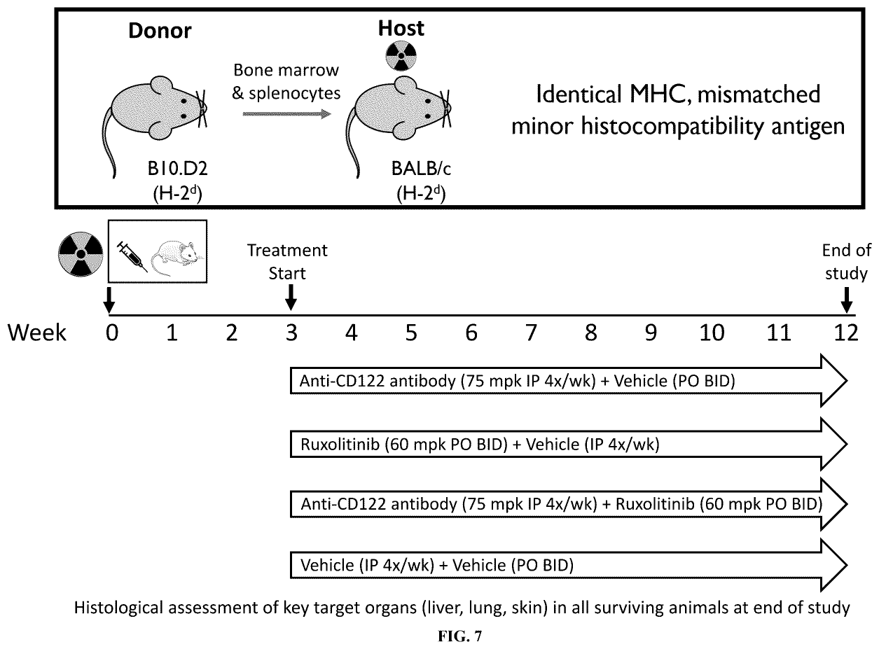

An allogeneic model of chronic GvHD according to the animal model design of Ramos et al (Ramos T L et al. Delayed administration of ixazomib modifies the immune response and prevents chronic graft-versus-host disease. Bone Marrow Transplant. 2021 December; 56(12):3049-3058) is investigated. In this model as shown in FIG. 7, B10.D2 mice serve as donor for bone marrow and splenocytes to be transplanted into irradiated BALB/c host mice. Both of these strains possess identical MHC (H-2^{d}), but are mismatched for minor histocompatibility antigens. In this model of cGvHD, there is a low mortality rate extending past 150 days post-transplant and cGvHD symptoms can typically appear starting at approximately 30 days post-transplant. Study design includes treatment start at Week 3 post-transplant and end of study is marked at Week 12 post-transplant. Each group has an n=20 of animals tested. Three test treatment groups include a) anti-CD122 antibody therapy (75 mpk delivered IP 4 times/week)+vehicle (PO BID), b) ruxolitinib (60 mpk PO BID)+vehicle (IP 4 times/week), and c) anti-CD122 antibody (75 mpk delivered IP 4 times/week)+ruxolitinib (60 mpk PO BID). One negative control group including vehicle (IP 4 times/week)+vehicle (PO BID) is also tested. Phenotypic cGvHD score and survival are assessed starting at Week 3 and ending at Week 12. This includes histological assessment of key target organs (including liver, lung, and skin) in all surviving animals. Results indicate that combination therapy of anti-CD122 antibody treatment via administration of an anti-CD122 antibody and JAK inhibition through administration of ruxolitinib produces superior prophylaxis and treatment of cGvHD symptoms than either anti-CD122 antibody therapy or ruxolitinib therapy alone. In this example, the anti-CD122 antibody administered to animals is an anti-CD122 antibody disclosed in the specification.

Detailed experiments protocols for testing this cGvHD model are listed below:

### Drugs and Treatment:

| Gr. | N | Strain | Active Agent | dose | Route | Schedule |
|---|---|---|---|---|---|---|
| 1 | 20 | BALB/c | Anti-CD122 | 75 mpk | IP | 4x/wk |
| | | | antibody | | | |
| 2 | 20 | BALB/c | Ruxolitinib | 60 mpk | PO | 2x/day |
| 3 | 20 | BALB/c | Anti-CD122 | 75 mpk and | IP and | 4x/wk and |
| | | | antibody and | 60 mpk | PO | 2x/day |
| | | | Ruxolitinib | | | |
| 4 | 20 | BALB/c | None | None | IP and | 4x/wk and |
| | | | (Vehicles only) | | PO | 2x/day |

#—Control Group

### Procedures:

BALB/c mice are randomized into treatment groups based on Day 1 bodyweight.
Female BALB/c mice are irradiated (5 Gy) and intravenously injected with B10.D2 donor cells on Day 1.
Cell injection volume is 200 μl of Bone Marrow cells and splenocytes mixture as described below in treatments. Total cell volume injection=200 μl in PBS
GvHD Scoring and body weight measurement: tiwk

GvHD Scoring:

| Criteria | Grade 0 | Grade 1 | Grade 2 |
|---|---|---|---|
| Weight Loss | <10% | ≥10%, <25% | ≥25% |
| Activity | Normal | Mild to moderately | Stationary until |
| Posture | Normal | Hunching only at rest | Severe gait, impaired |
| Fur Texture | Normal | Mild to moderate | Severe ruffling/poor |
| Skin Integrity | Normal | Obvious areas of | Scaling of paws |
| | | denuded skin | and/or tail |

Histological assessment key target organs, including liver, lung, and skin, are conducted in all surviving animals at study termination.

### Example 4: Acute GvHD Study for Dose-Ranging Investigation of Anti-CD122 Antibody

A xenograft GvHD mouse model is utilized to study human T and NK cell-mediated aGvHD and the therapeutic effects of anti-CD122 antibody treatment therein. Female NCG-hIL-15 mice are irradiated at a dosage of 100 cGy on Day 1 of the protocol and human peripheral blood mononuclear cells (PBMCs) are transplanted via injection at a dosage of 1×10⁷ cells/200 μL/mice via IV administration into the irradiated mice. PBMCs are immune cells that originate in the bone marrow and are secreted into the peripheral circulation forming critical components of the immune system. PBMCs are involved in humoral and cell-mediated immune function. As seen from the study design in FIG. 1, treatment with an anti-CD122 antibody is begun on Day 5 following transplant. Groups of mice are used for each test category in sufficient number of animals to evaluate potential differences between groups. A vehicle group serves as a negative control with no treatment administered (only a combination of vehicle treatments) following transplant. A positive control group of mice treated with 60 mcg each of ruxolitinib twice daily by oral administration is included in the study design. Ruxolitinib-treated animals are treated via oral administration of ruxolitinib twice daily on D5, D6, D7, D8, D9, D10, D11, D12, D13, and D14. Three groups of anti-CD122 antibody treated animals are tested using a) 75 milligram per kilogram of body weight (mpk) of antibody, b) 125 mpk of antibody, and c) 175 mpk of antibody, respectively. Animals are treated via intraperitoneal (IP) injection for administration of an anti-CD122 antibody as described herein once daily on D5, D6, D7, D8, D9, D10, D11, D12, D13, and D14. The study concludes on Day 14.

Test subject survival at study conclusion is examined for determining improvement in survival in the animals treated with 75, 125, or 175 mpk of the anti-CD122 antibody compared to negative control subjects and positive control subjects. These results are assessed to examine efficacy of anti-CD122 antibody therapy using an antibody described in this specification as an effective therapeutic strategy for aGvHD.

### Example 5: Acute GvHD Study Comparing Single and Combination Therapies

A xenograft GvHD mouse model was utilized to study human T and NK cell-mediated aGvHD and the therapeutic effects of anti-CD122 antibody treatment in single or combination therapy approaches. Female NCG-hIL-15 mice were irradiated at a dosage of 100 cGy on Day 1 of the protocol and human peripheral blood mononuclear cells (PBMCs) were transplanted via injection at a dosage of 1×10⁷ cells/200 μL/mice via IV administration into the irradiated mice according to the same xenograft GvHD mouse model design as in Example 1.

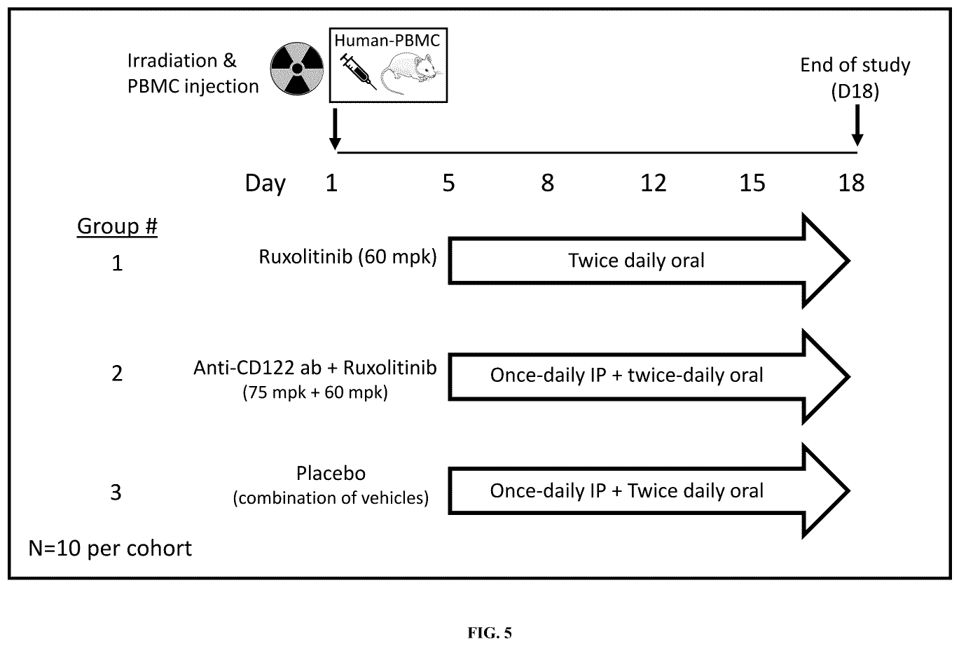

Treatment according to 3 different grouping of animals was begun on Day 5 following transplant, as shown in the study design of FIG. 5. Groups of a sufficient number of mice were used for each test category in order to evaluate a significant difference between test groups. A single treatment group, Group #1, received ruxolitinib (at a dosage of 60 mpk) twice-daily by oral administration beginning on Day 5 and ending on Day 18. Days of treatment are D5, D6, D7, D8, D9, D10, D11, D12, D13, D14, D15, D16, D17, and D18. A combination therapy group, Group #2, received ruxolitinib (at a dosage of 60 mpk) twice-daily by oral administration beginning on Day 5 and ending on Day 18. Days of treatment using ruxolitinib in Group #2 were D5, D6, D7, D8, D9, D10, D1I, D12, D13, D14, D15, D16, D17, and D18. Group #2 animals separately receive anti-CD122 antibody therapy (at a dosage of 75 mpk) once-daily by IP administration. Days of treatment using anti-CD122 antibody in Group #2 were D5, D6, D7, D8, D9, D10, D11, D12, D13, D14, D15, D16, D17, and D18. A vehicle group, Group #3, served as a negative control with vehicle-only administered following transplant received twice daily oral placebo (vehicle only) beginning on Day 5 and ending on Day 18. Days of oral placebo treatment were D5, D6, D7, D8, D9, D10, D11, D12, D13, D14, D15, D16, D17, and D18. This vehicle group also separately received once daily IP placebo (vehicle only) on D5, D6, D7, D8, D9, D10, D11, D12, D13, D14, D15, D16, D17, and D18. In this example, the anti-CD122 antibody administered to animals is an anti-CD122 antibody described in the specification.

Day 18 was the end of study day for all treatment groups. Animals from all treatment groups were assessed daily beginning on Day 1 and ending on Day 18 for survival following transplant. A comparison of survival rate between Group #1 (single treatment group with ruxolitinib monotherapy), Group #2 (combination therapy group with ruxolitinib and anti-CD122 antibody combination treatment), and Group #3 (placebo group serving as negative control) was undertaken to evaluate treatment effects on subject survival rate daily throughout the study and to evaluate overall survival rate at the end of the study. At the end of the study, tissue was collected from all surviving animals for histological assessment of key target organs. These key target organs include liver, lung, and skin. A histological and phenotypic analysis of collected tissue is undertaken to determine the presence and extent of aGvHD symptoms at an organ level, a tissue level, a cellular level, and a molecular level. A comparison of histological findings between treatment groups is undertaken to determine which treatment groups yield the most favorable outcomes.

The study was evaluated to determine efficacy of a therapeutic benefit for survival in this model of aGvHD for animals receiving combination therapy of ruxolitinib treatment and anti-CD122 antibody treatment compared with either ruxolitinib single therapy or compared with placebo-treatment. Combination therapy for aGvHD comprising administering anti-CD122 antibody and ruxolitinib was evaluated to determine an extent of improvement in survival rate compared with ruxolitinib monotherapy.

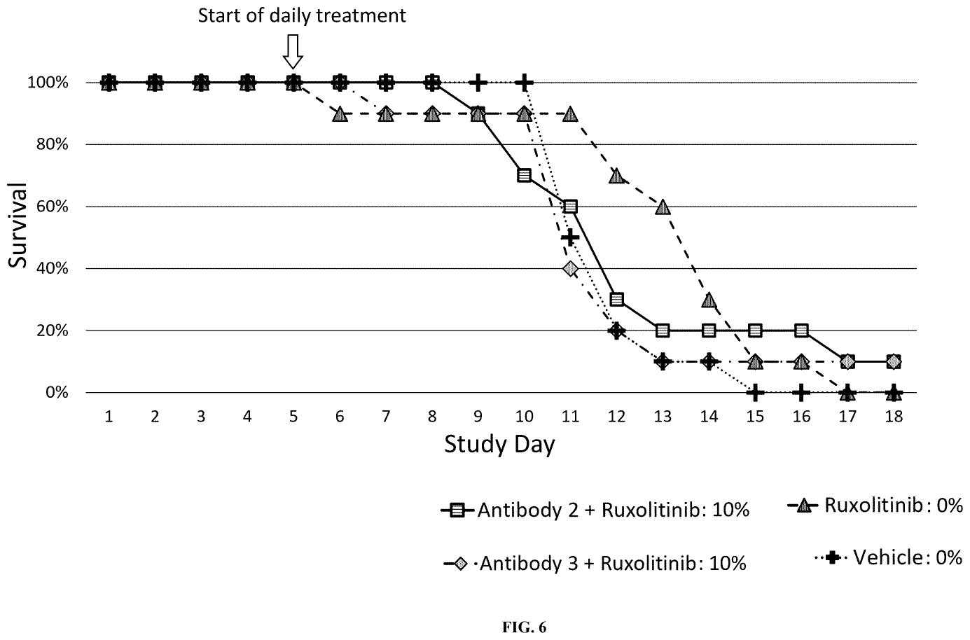

FIG. 6 shows a graph of survival duration in the acute GvHD mouse model described in this example comparing effectiveness of i) anti-CD122 antibody (Antibody 2 as described herein) plus ruxolitinib treatment, ii) anti-CD122 antibody (Antibody 3 as described herein) plus ruxolitinib treatment, and iii) ruxolitinib monotherapy, versus iv) vehicle-treated control using xenotransplant from a specific donor of human PBMCs. Survival rates of different groups on Day 18 are as follows:

Group #1 (single treatment group with ruxolitinib monotherapy): 0% survival (0/10), Group #2a (combination therapy group with ruxolitinib and Antibody 2 combination treatment): 10% survival (1/10), Group #2b (combination therapy group with ruxolitinib and Antibody 3 combination treatment): 10% survival (1/10), and Group #3 (placebo group serving as negative control): 0% survival (0/10). Table 22 lists survival rates of different groups on Day 18.

TABLE 22
Survival rates of different groups on Day 18

| | Average | Median | P values |
|---|---|---|---|
| | survival | survival | (vs. |
| Group | time (days) | (days) | Group #3) |
| Group #1 | 13.20 ± 0.93 | 14 | 0.016* |
| (ruxolitinib | | | |
| monotherapy) | | | |
| Group #2a | 12.40 ± 0.93 | 12 | 0.136 |
| (combination | | | |
| therapy: | | | |
| Antibody 2 + | | | |
| ruxolitinib) | | | |
| Group #2b | 11.70 ± 0.86 | 11 | 0.204 |
| (combination | | | |
| therapy: | | | |
| Antibody 3 + | | | |
| ruxolitinib) | | | |
| Group #3 | 11.00 ± 0.49 | 10.5 | — |
| (Placebo - | | | |
| vehicle control) | | | |

Data shown as Mean ± SD. Statistics were performed using Log-rank test compared to Group #3, *: P ≤ 0.05. Groups #2a and #2b were the only test groups in this example to have at least one surviving animal at end of study (Day 18).

### Example 6: Anti-CD122 Antibody Treatment in aGvHD Patients

A bicentric non-controlled fixed-dose treatment is performed to evaluate the efficacy of an antibody directed against CD122 in patients with aGvHD. This is an interventional study type, the primary purpose of which is treatment. In this study, anti-CD122 antibody monotherapy and anti-CD122 antibody combination therapy with ruxolitinib administration is evaluated. A total of 40 adult patients with acute steroid-resistant GVHD are treated for a 12 months period. Below inclusion and exclusion criteria for selection of patients are used:

### Inclusion Criteria:

Patients at least 18 years of age.
Patients suffering from acute GVHD which is staged Grade II-IV according to the modified Glucksberg Criteria and progressing after 3 days, or not improving after 7 days, of methylprednisolone at a dose of 2 mg/kg per day.
Patients or an impartial witness (in case the patient is capable to provide verbal consent but not capable to sign the informed consent) have given written informed consent.

### Exclusion Criteria:

Patients with signs or symptoms suggestive of chronic GvHD.
Patients receiving concomitant investigational therapeutics for acute GvHD, including investigational agents used for GvHD prophylaxis, at the time of enrollment.
Patients participating in a clinical trial with another investigational product within 30 days prior to providing informed consent.

Number of arms: 2 (i: anti-CD122 antibody monotherapy, and ii: anti-CD122 antibody combination therapy with ruxolitinib). 20 patients assigned to each arm. Monotherapy treatment comprises a standard dose infusion of a pharmaceutical composition comprising an anti-CD122 antibody described herein selected from G1-G20 (dosage range selected from 6-2000 mg fixed dose) given 48-hours apart by i.v. administration over a 4-hour period. Combination treatment comprises a standard dose infusion of a pharmaceutical composition comprising an anti-CD122 antibody described herein selected from G1-G20 (dosage range selected from 6-2000 mg fixed dose) given 48-hours apart by i.v. administration over a 4-hour period and a separate administration of ruxolitinib (dosage and administration frequency range from 5 mg QD to 10 mg BID). Dose reductions may be used to manage side effects: 10 mg BID may be reduced to 5 mg BID; 5 mg BID may be reduced to 5 mg QD. Tapering may be considered after 6 months of treatment as clinically indicated. Taper ruxolitinib by 1 dose level approximately every 8 weeks (e.g., 10 mg BID to 5 mg BID to 5 mg QD). The follow-up period is 6 months. This is an open label study with no masking.

The primary objective is to determine the efficacy of the anti-CD122 antibody monotherapy, or the anti-CD122 antibody in combination with ruxolitinib, 4 weeks after the first infusion (Day 28), in inducing an objective clinical response in patients with acute GvHD refractory to standard first line corticosteroid therapy.

### The Secondary Objectives are:

To evaluate the overall safety and efficacy of anti-CD122 antibody during the first 6 months after initiation of therapy.
To determine the pharmacokinetic profile of anti-CD122 antibody
To determine the immunogenicity of anti-CD122 antibody

### The Exploratory Objectives are:

To study the specificity and kinetics of the treatment-induced depletion and subsequent repopulation of certain lymphocyte subsets
To evaluate diagnostic and predictive aGvHD biomarkers relative to treatment outcomes

### Primary Outcome Measure: AGvHD Response Rate.

The aGvHD response rate at 4 weeks after the first injection of anti-CD122 antibody, being defined as the fraction of patients showing a complete or partial response (CR or PR) within a time frame up to Day 28.

### Secondary Outcome Measures:

1) Safety and tolerability of anti-CD122 antibody treatment. The safety and tolerability of anti-CD122 antibody administration as assessed by evaluating Dose Limiting Toxicities (DLT's), adverse and serious adverse events reported during 6 months after initiation of treatment within a time frame up to up to 6 months after initiation of treatment.
2) Very good partial response rate. The proportion of patients achieving a very good partial response rate (VGPR) of their aGvHD at 4 weeks after the first infusion (Day 28).
3) Acute GVHD relapse rate during 6 months after initiation of therapy.
4) Incidence of development of chronic GvHD during 6 months after initiation of therapy.
5) Overall survival and progression-free survival during 6 months after initiation of therapy.
6) Pharmacokinetic profile of anti-CD122 antibody:
   a. Areas under the time-concentration curves (AUC);
   b. Peak concentration (Cmax);
   c. Time to peak concentration (Tmax);
   d. Terminal-phase elimination half-life (t½);
   e. Apparent Clearance (CL/F);
   f. Steady-state volume of distribution (Vss/F)
7) The occurrence of treatment-induced cytokine release. The occurrence of treatment-induced cytokine release, as determined by measurement of interleukin-2 (IL-2), IL-4, IL-5, IL-6, IL-8, IL-10, tumor necrosis factor-alpha (TNF-α), and interferon-gamma (IFN-γ) serum levels at t=0 (pre-dose), approximately 1 and 4 hours after starting of each infusion.

### Other Outcome Measures:

1) Kinetics and specificity of treatment-induced T cell and natural killer cell (NK cell) depletion. This is determined by the flow cytometric assessment of the number of T-cells, B-cells and NK-cells during the first 4 weeks after initiation of treatment.
2) Composition and evolution of T-, B- and NK-cell compartments. This is determined by the flow cytometric phenotyping of lymphocyte subsets for determine the composition and evolution of the T-, B-, and NK-cells compartments at pretreatment and at 4 weeks, 3 and 6 months after the first infusion.
3) Composition and evolution of T-cell receptor (TCR) Vbeta group at pretreatment and at 4 weeks, 3 and 6 months after the first infusion.
4) The identification and evolution of host-reactive T-cell clones at pretreatment and at 4 weeks, 3 and 6 months after the first infusion.
5) aGvHD biomarkers. Measurement of diagnostic and predictive aGvHD biomarkers relative to treatment outcomes, including citrulline, C reactive protein (CRP), elafin, IL8, tumor necrosis factor receptor 1 (TNFR1), interleukin 2 receptor-alpha (IL-2Ralpha), hepatocyte growth factor (HGF), and Reg3alpha at pre-treatment, Day 14, Day 28, Day 90, and Day 180.

### Summary of Evaluation of Primary Objective:

Evaluation of the primary object determines the efficacy of anti-CD122 monotherapy and anti-CD122 combination therapy with ruxolitinib and allows comparison of respective efficacy of each treatment arm.

### Example 7: Anti-CD122 Antibody Treatment in cGvHD Patients

This study aims to determine how well the anti-CD122 antibody used for treatment works in treating chronic GvHD in patients that have not responded to treatment after at least two prior therapies. The anti-CD122 antibody blocks functions of a protein (CD122) that stimulates the body's immune system. By blocking this protein, the investigators may reduce the symptoms of chronic GvHD.

Primary Objective: Efficacy is determined by the proportion of patients with failure free survival (FFS) at 6 month.

### Secondary Objectives:

1) Patients achieving a complete response (CR) or partial response (PR) at 6 months based on clinician judged response.
2) Patients achieving a CR or PR by objective response measures at 6 months.
3) Failure-free survival (FFS) at 1 year.
4) Relative change in steroid dose from enrollment to 6 months. Prednisone is not a pre-specified intervention; however, some patients may take prednisone while on this study.

Time to study secondary objectives: from baseline to 6 months after initiation of treatment.

### Study Outline:

Patients receive the pharmaceutical composition comprising the selected anti-CD122 antibody (selected from antibodies GT-G20) intravenously (IV) over 1 hour every 2 weeks for 12 weeks (weeks 1, 3, 5, 7, 9, and 11) and then every 4 weeks for 12 weeks (weeks 13, 17, and 21). After completion of study treatment, patients are followed up at 3 and 6 months. This is an interventional study type, the primary purpose of which is supportive care of enrolled patients. Interventional study model: Single group assignment. Number of arms: 1. No masking is performing in the study (open label). Fixed dosage of pharmaceutical composition comprising the anti-CD122 antibody (selected dosage range between 6-5000 mg) is delivered with each administration.

Arm 1: Patients receive the pharmaceutical composition comprising the selected anti-CD122 antibody IV over 1 hour every 2 weeks for 12 weeks (weeks 1, 3, 5, 7, 9, and 11) and then every 4 weeks for 12 weeks (weeks 13, 17, and 21).

### Other Outcome Measured Assessed:

1) B-cell subsets (assessed up to Week 21).
2) Tumor necrosis factor superfamily, member 13b (BAFF) levels (assessed up to Week 21).
3) T-cell subsets (assessed up to Week 21).

For selection of patients to be treated with anti-CD122 antibody, below inclusion and exclusion criteria has been utilized:

### Inclusion Criteria:

Patients >18 years of age at time of baseline assessment
Subject has moderate or severe overlap cGvHD according to National Institutes of Health (NIH) criteria
Active cGvHD despite treatment with at least two immunosuppressive treatments (not including GvHD prophylaxis) in the past year
Subject underwent an allogeneic stem cell transplantation at least 6 months prior to enrollment in study
Subject has not begun any new systemic immunosuppressive therapies within 2 weeks prior to enrollment
Subject meets the following medication restriction requirements and agrees to follow medication restrictions during the study; the following concomitant medications are not allowed: cyclophosphamide, abatacept, etanercept, adalimumab infliximab, golimumab, tofacitinib, and alemtuzumab; these medications also cannot have been used for at least 4 half-lives prior to enrollment
Subject agrees to comply with the study requirements and agrees to come to the clinic for required study visits during the study.

### Exclusion Criteria:

Donor lymphocyte infusion in the preceding 100 days
Evidence of malignancy within 6 months of study enrollment; this is defined as clear morphologic, radiologic or molecular evidence of disease; mixed chimerism is allowed at the discretion of the clinician
Treatment with any non-Food and Drug Administration (FDA) approved agent within 4 weeks (or 4 half-lives of the investigational drug, whichever is longer) of study enrollment
Serum creatinine >1.6 mg/dL (141 μmol/L) in females and >1.9 mg/dL (168 μmol/L) in males; patients with serum creatinine values exceeding these limits are eligible for the study if their estimated glomerular filtration rates (GFR) are >30 ml/min/1.73 m\hat{ }2
Alanine aminotransferase (ALT) or aspartate aminotransferase (AST) >1.5 times upper limit of normal (ULN)
Total bilirubin >upper limit of normal (ULN)
Absolute neutrophil count <1.5×10\hat{ }9/L (1500/mm\hat{ }3)

Summary of evaluation of primary objective: The proportion of patients with failure free survival (FFS) at 6 months is calculated to assess treatment efficacy compared to a known proportion of subjects with similar symptom criteria who are not undergoing the treatment listed in this study design.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

### ASPECTS

Aspect 1: A method of preventing or treating graft versus host disease (GvHD) in a subject in need thereof, the method comprising: administering to the subject an effective amount of an anti-CD122 antibody, or its antigen-binding fragment thereof, comprising i) a variable heavy chain (VH) domain and ii) a variable light chain (VL) domain, wherein the VH domain comprises an HCDR1 sequence comprising a sequence selected from SEQ ID NOs: 110-115, an HCDR2 sequence comprising a sequence selected from SEQ ID NOs: 116-123, and an HCDR3 sequence comprising a sequence selected from SEQ ID NOs: 124-130, and the VL domain comprises an LCDR1 sequence comprising a sequence selected from SEQ ID NOs: 131-140, an LCDR2 sequence comprising a sequence selected from SEQ ID NOs: 141-146, and an LCDR3 sequence comprising a sequence selected from SEQ ID NOs: 147-151.

Aspect 2: The method of aspect 1, wherein the HCDR1 sequence comprises SEQ ID NO: 110, the HCDR2 sequence comprises SEQ ID NO: 116, the HCDR3 sequence comprises SEQ ID NO: 124, the LCDR1 sequence comprises SEQ ID NO: 131, the LCDR2 sequence comprises SEQ ID NO: 141, and the LCDR3 sequence comprises SEQ ID NO: 147.

Aspect 3: The method of aspect 1, wherein the HCDR1 sequence comprises SEQ ID NO: 111, the HCDR2 sequence comprises SEQ ID NO: 116, the HCDR3 sequence comprises SEQ ID NO: 125, the LCDR1 sequence comprises SEQ ID NO: 132, the LCDR2 sequence comprises SEQ ID NO: 141, and the LCDR3 sequence comprises SEQ ID NO: 147.

Aspect 4: The method of aspect 1, wherein the HCDR1 sequence comprises SEQ ID NO: 111, the HCDR2 sequence comprises SEQ ID NO: 116, the HCDR3 sequence comprises SEQ ID NO: 125, the LCDR1 sequence comprises SEQ ID NO: 133, the LCDR2 sequence comprises SEQ ID NO: 141, and the LCDR3 sequence comprises SEQ ID NO: 147.

Aspect 5: The method of aspect 1, wherein the HCDR1 sequence comprises SEQ ID NO: 111, the HCDR2 sequence comprises SEQ ID NO: 117, the HCDR3 sequence comprises SEQ ID NO: 125, the LCDR1 sequence comprises SEQ ID NO: 132, the LCDR2 sequence comprises SEQ ID NO: 142, and the LCDR3 sequence comprises SEQ ID NO: 148.

Aspect 6: The method of aspect 1, wherein the HCDR1 sequence comprises SEQ ID NO: 110, the HCDR2 sequence comprises SEQ ID NO: 118, the HCDR3 sequence comprises SEQ ID NO: 124, the LCDR1 sequence comprises SEQ ID NO: 134, the LCDR2 sequence comprises SEQ ID NO: 143, and the LCDR3 sequence comprises SEQ ID NO: 149.

Aspect 7: The method of aspect 1, wherein the HCDR1 sequence comprises SEQ ID NO: 112, the HCDR2 sequence comprises SEQ ID NO: 119, the HCDR3 sequence comprises SEQ ID NO: 126, the LCDR1 sequence comprises SEQ ID NO: 135, the LCDR2 sequence comprises SEQ ID NO: 142, and the LCDR3 sequence comprises SEQ ID NO: 147.

Aspect 8: The method of aspect 1, wherein the HCDR1 sequence comprises SEQ ID NO: 113, the HCDR2 sequence comprises SEQ ID NO: 120, the HCDR3 sequence comprises SEQ ID NO: 124, the LCDR1 sequence comprises SEQ ID NO: 136, the LCDR2 sequence comprises SEQ ID NO: 143, and the LCDR3 sequence comprises SEQ ID NO: 148.

Aspect 9: The method of aspect 1, wherein the HCDR1 sequence comprises SEQ ID NO: 110, the HCDR2 sequence comprises SEQ ID NO: 119, the HCDR3 sequence comprises SEQ ID NO: 127, the LCDR1 sequence comprises SEQ ID NO: 137, the LCDR2 sequence comprises SEQ ID NO: 143, and the LCDR3 sequence comprises SEQ ID NO: 149.

Aspect 10: The method of aspect 1, wherein the HCDR1 sequence comprises SEQ ID NO: 110, the HCDR2 sequence comprises SEQ ID NO: 121, the HCDR3 sequence comprises SEQ ID NO: 128, the LCDR1 sequence comprises SEQ ID NO: 138, the LCDR2 sequence comprises SEQ ID NO: 144, and the LCDR3 sequence comprises SEQ ID NO: 148.

Aspect 11: The method of aspect 1, wherein the HCDR1 sequence comprises SEQ ID NO: 114, the HCDR2 sequence comprises SEQ ID NO: 122, the HCDR3 sequence comprises SEQ ID NO: 129, the LCDR1 sequence comprises SEQ ID NO: 139, the LCDR2 sequence comprises SEQ ID NO: 145, and the LCDR3 sequence comprises SEQ ID NO: 150.

Aspect 12: The method of aspect 1, wherein the HCDR1 sequence comprises SEQ ID NO: 115, the HCDR2 sequence comprises SEQ ID NO: 123, the HCDR3 sequence comprises SEQ ID NO: 130, the LCDR1 sequence comprises SEQ ID NO: 140, the LCDR2 sequence comprises SEQ ID NO: 146, and the LCDR3 sequence comprises SEQ ID NO: 151.

Aspect 13: The method of any one of aspects 1-12, wherein the VH domain comprises at least 80%, at least 85%, at least 90%, at least 95%, at least 97%, at least 99%, or 100% sequence identity to a sequence selected from SEQ ID NOs: 152-158.

Aspect 14: The method of any one of aspects 1-13, wherein the VL domain comprises at least 80%, at least 85%, at least 90%, at least 95%, at least 97%, at least 99%, or 100% identity to a sequence selected from SEQ ID NOs: 159-165.

Aspect 15: The method of any one of aspects 1-14, wherein the VH domain comprises a portion of a heavy chain comprising at least 80%, at least 85%, at least 90%, at least 95% sequence identity to a sequence selected from SEQ ID NOs: 177-196.

Aspect 16: The method of any one of aspects 1-15, wherein the VL domain comprises a portion of a light chain comprising at least 80%, at least 85%, at least 90%, at least 95% sequence identity to a sequence selected from SEQ ID NOs: 197-199.

Aspect 17: The method of any one of aspects 1-16, wherein the anti-CD122 antibody is a monoclonal antibody.

Aspect 18: The method of any one of aspects 1-17, wherein the anti-CD122 antibody is a humanized antibody.

Aspect 19: The method of any one of aspects 1-18, wherein the anti-CD122 antibody or its antigen-binding fragment thereof comprises IgG-scFv, IgA, IgM, IgE antibody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

Aspect 20: The method of any one of aspects 1-19, wherein the anti-CD122 antibody interferes with Interleukin-15 (IL-15) binding to the intermediate affinity IL-βγ receptor.

Aspect 21: The method of any one of aspects 1-20, wherein the anti-CD122 antibody diminishes or disrupts IL-15-induced signal transduction.

Aspect 22: The method of any one of aspects 1-21, wherein the anti-CD122 antibody interferes with Interleukin-2 (IL-2) binding to the intermediate affinity IL-βγ receptor.

Aspect 23: The method of any one of aspects 1-22, wherein the anti-CD122 antibody diminishes or disrupts IL-2-induced signal transduction mediated through the intermediate affinity IL-βγ receptor.

Aspect 24: The method of any one of aspects 1-23, wherein the GvHD is acute graft versus host disease (aGvHD).

Aspect 25: The method of any one of aspects 1-24, wherein the administering an effective amount of the anti-CD122 antibody delays an onset of one or more symptoms of aGvHD in the subject.

Aspect 26: The method of any one of aspects 1-25, wherein the administering an effective amount of the anti-CD122 antibody alleviates one or more symptoms of aGvHD in the subject.

Aspect 27: The method of aspect 25 or 26, wherein the one or more symptoms of aGvHD comprises itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, or weight loss.

Aspect 28: The method of any one of aspects 1-23, wherein the GvHD is chronic graft versus host disease (cGvHD).

Aspect 29: The method of aspect 28, wherein the administering an effective amount of the anti-CD122 antibody delays an onset of one or more symptoms of chronic GvHD (cGvHD) in the subject.

Aspect 30: The method of aspect 28 or 29, wherein the administering an effective amount of the anti-CD122 antibody alleviates one or more symptoms of cGvHD in the subject.

Aspect 31: The method of aspect 29 or 30, wherein the one or more symptoms of cGvHD comprises skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, or changes in hair texture.

Aspect 32: The method of any one of aspects 1-31, wherein the administering an effective amount of the anti-CD122 antibody increases the survival rate of the subject.

Aspect 33: The method of any one of aspects 1-32, wherein the anti-CD122 antibody is administered systemically, locally, intradermally, subcutaneously, or topically.

Aspect 34: The method of aspect 33, wherein the anti-CD122 antibody administered systemically is administered by intravenous injection.

Aspect 35: The method of any one of aspects 1-34, wherein the method further comprises administering to the subject an effective amount of a JAK inhibitor, thereby preventing or treating GvHD in the subject.

Aspect 36: The method of aspect 35, wherein the anti-CD122 antibody and the JAK inhibitor are co-administered.

Aspect 37: The method of aspect 35 or 36, wherein the anti-CD122 antibody and the JAK inhibitor are administered separately or sequentially.

Aspect 38: The method of any one of aspects 35-37, wherein the JAK inhibitor is selected from ruxolitinib, abrocitinib, baricitinib, delgocitinib, fedratinib, filgotinib, oclacitinib, pacritinib, peficitinib, tofacitinib, itacitinib and upadacitinib.

Aspect 39: The method of any one of aspects 35-38, wherein the JAK inhibitor is ruxolitinib.

Aspect 40: The method of any one of aspects 35-39, wherein the JAK inhibitor is administered first and the anti-CD122 antibody is administered second.

Aspect 41: The method of any one of aspects 35-39, wherein the administering an effective amount of the anti-CD122 antibody and the JAK inhibitor delays an onset of one or more symptoms of aGvHD or cGvHD in the subject.

Aspect 42: The method of any one of aspects 35-41, wherein the administering an effective amount of the anti-CD122 antibody and the JAK inhibitor alleviates one or more symptoms of aGvHD or cGvHD in the subject.

Aspect 43: The method of any one of aspects 35-42, wherein the administering an effective amount of the anti-CD122 antibody and the JAK inhibitor increases the survival rate of the subject.

Aspect 44: The method of any one of aspects 35-43, wherein the administering an effective amount of the anti-CD122 antibody and the JAK inhibitor increases the survival rate of the subject compared with a subject treated with a JAK inhibitor as a monotherapy.

Aspect 45: The method of any one of aspects 35-44, wherein the anti-CD122 antibody and the JAK inhibitor are administered by the same route of administration.

Aspect 46: The method of any one of aspects 35-45, wherein the anti-CD122 antibody and the JAK inhibitor are administered by separate routes of administration.

Aspect 47: The method of any one of aspects 35-46, wherein the anti-CD122 antibody is administered systemically, locally, intradermally, subcutaneously, or topically, and wherein the JAK inhibitor is administered systemically, locally, intradermally, subcutaneously, or topically.

Aspect 48: The method of aspect 47, wherein the anti-CD122 antibody administered systemically is administered by intravenous injection or intraperitoneal injection.

Aspect 49: The method of aspect 47 or 48, wherein the JAK inhibitor administered systemically is administered by intravenous injection, by enteral administration, or through inhalation.

Aspect 50: The method of aspect 49, wherein the JAK inhibitor administered by enteral administration is administered orally.

Aspect 51: A method of treating graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 152, and an LCDR1, LCDR2, LCDR3 of a light chain variable domain comprising the amino acid sequence set forth in SEQ ID NO: 159, wherein the administering treats one or more symptoms of acute GvHD or chronic GvHD.

Aspect 52: A method of preventing graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 152, and an LCDR1, LCDR2, LCDR3 of a light chain variable domain comprising the amino acid sequence set forth in SEQ ID NO: 159, wherein the administering prevents one or more symptoms of acute GvHD or chronic GvHD.

Aspect 53: A method of treating graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 160, wherein the administering treats one or more symptoms of acute GvHD or chronic GvHD.

Aspect 54: A method of preventing graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 160, wherein the administering prevents one or more symptoms of acute GvHD or chronic GvHD.

Aspect 55: A method of treating graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 161, wherein the administering treats one or more symptoms of acute GvHD or chronic GvHD.

Aspect 56: A method of preventing graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 153, and an LCDR1, LCDR2, LCDR3 of a light chain variable domain comprising the amino acid sequence set forth in SEQ ID NO: 161, wherein the administering prevents one or more symptoms of acute GvHD or chronic GvHD.

Aspect 57: A method of treating graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 156, and an LCDR1, LCDR2, LCDR3 of a light chain variable domain comprising the amino acid sequence set forth in SEQ ID NO: 163, wherein the administering treats one or more symptoms of acute GvHD or chronic GvHD.

Aspect 58: A method of preventing graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 156, and an LCDR1, LCDR2, LCDR3 of a light chain variable domain comprising the amino acid sequence set forth in SEQ ID NO: 163, wherein the administering prevents one or more symptoms of acute GvHD or chronic GvHD.

Aspect 59: A method of treating graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 158, and an LCDR1, LCDR2, LCDR3 of a light chain variable domain comprising the amino acid sequence set forth in SEQ ID NO: 165, wherein the administering treats one or more symptoms of acute GvHD or chronic GvHD.

Aspect 60: A method of preventing graft versus host disease (GvHD) in a subject in need thereof, the method comprising: administering to the subject an effective amount of an anti-CD122 antibody, or its antigen-binding fragment thereof, comprising an HCDR1, HCDR2, HCDR3 of a heavy chain variable domain comprising the amino acid sequence set forth in SEQ ID NO: 158, and an LCDR1, LCDR2, LCDR3 of a light chain variable domain comprising the amino acid sequence set forth in SEQ ID NO: 165, wherein the administering prevents one or more symptoms of acute GvHD or chronic GvHD.

Aspect 61: A method of preventing or treating graft versus host disease (GvHD) in a subject in need thereof, the method comprising:
administering to the subject an effective amount of an anti-CD122 antibody, or its antigen-binding fragment thereof, comprising i) a variable heavy chain (VH) domain and ii) a variable light chain (VL) domain, wherein the VH domain comprises an HCDR1 sequence comprising a sequence selected from SEQ ID NOs: 1-11, an HCDR2 sequence comprising a sequence selected from SEQ ID NOs: 12-23, and an HCDR3 sequence comprising a sequence selected from SEQ ID NOs: 24-36, and the VL domain comprises an LCDR1 sequence comprising a sequence selected from SEQ ID NOs: 37-47, an LCDR2 sequence comprising a sequence selected from GTS, TTS, YTS, WAS, KAS, GAT, YAS or STS, and an LCDR3 sequence comprising a sequence selected from SEQ ID NOs: 56-67.

Aspect 62: The method of aspect 61, wherein:
the HCDR1 sequence comprises SEQ ID NO: 7, the HCDR2 sequence comprises SEQ ID NO: 18, the HCDR3 sequence comprises SEQ ID NO: 30, the LCDR1 sequence comprises SEQ ID NO: 43, the LCDR2 sequence comprises YTS, and the LCDR3 sequence comprises SEQ ID NO: 62,
the HCDR1 sequence comprises SEQ ID NO: 1, the HCDR2 sequence comprises SEQ ID NO: 12, the HCDR3 sequence comprises SEQ ID NO: 24, the LCDR1 sequence comprises SEQ ID NO: 37, the LCDR2 sequence comprises GTS, and the LCDR3 sequence comprises SEQ ID NO: 56,
the HCDR1 sequence comprises SEQ ID NO: 2, the HCDR2 sequence comprises SEQ ID NO: 13, the HCDR3 sequence comprises SEQ ID NO: 25, the LCDR1 sequence comprises SEQ ID NO: 38, the LCDR2 sequence comprises TTS, and the LCDR3 sequence comprises SEQ ID NO: 57,
the HCDR1 sequence comprises SEQ ID NO: 3, the HCDR2 sequence comprises SEQ ID NO: 14, the HCDR3 sequence comprises SEQ ID NO: 26, the LCDR1 sequence comprises SEQ ID NO: 39, the LCDR2 sequence comprises YTS, and the LCDR3 sequence comprises SEQ ID NO: 58,
the HCDR1 sequence comprises SEQ ID NO: 4, the HCDR2 sequence comprises SEQ ID NO: 15, the HCDR3 sequence comprises SEQ ID NO: 27, the LCDR1 sequence comprises SEQ ID NO: 40, the LCDR2 sequence comprises WAS, and the LCDR3 sequence comprises SEQ ID NO: 59,
the HCDR1 sequence comprises SEQ ID NO: 5, the HCDR2 sequence comprises SEQ ID NO: 16, the HCDR3 sequence comprises SEQ ID NO: 28, the LCDR1 sequence comprises SEQ ID NO: 41, the LCDR2 sequence comprises YTS, and the LCDR3 sequence comprises SEQ ID NO: 60,
the HCDR1 sequence comprises SEQ ID NO: 6, the HCDR2 sequence comprises SEQ ID NO: 17, the HCDR3 sequence comprises SEQ ID NO: 29, the LCDR1 sequence comprises SEQ ID NO: 42, the LCDR2 sequence comprises KAS, and the LCDR3 sequence comprises SEQ ID NO: 61,
the HCDR1 sequence comprises SEQ ID NO: 8, the HCDR2 sequence comprises SEQ ID NO: 19, the HCDR3 sequence comprises SEQ ID NO: 31, the LCDR1 sequence comprises SEQ ID NO: 44, the LCDR2 sequence comprises YTS, and the LCDR3 sequence comprises SEQ ID NO: 63,
the HCDR1 sequence comprises SEQ ID NO: 9, the HCDR2 sequence comprises SEQ ID NO: 20, the HCDR3 sequence comprises SEQ ID NO: 32, the LCDR1 sequence comprises SEQ ID NO: 45, the LCDR2 sequence comprises GAT, and the LCDR3 sequence comprises SEQ ID NO: 64,
the HCDR1 sequence comprises SEQ ID NO: 1, the HCDR2 sequence comprises SEQ ID NO: 21, the HCDR3 sequence comprises SEQ ID NO: 33, the LCDR1 sequence comprises SEQ ID NO: 37, the LCDR2 sequence comprises GTS, and the LCDR3 sequence comprises SEQ ID NO: 65,
the HCDR1 sequence comprises SEQ ID NO: 1, the HCDR2 sequence comprises SEQ ID NO: 21, the HCDR3 sequence comprises SEQ ID NO: 34, the LCDR1 sequence comprises SEQ ID NO: 37, the LCDR2 sequence comprises GTS, and the LCDR3 sequence comprises SEQ ID NO: 65,
the HCDR1 sequence comprises SEQ ID NO: 10, the HCDR2 sequence comprises SEQ ID NO: 22, the HCDR3 sequence comprises SEQ ID NO: 35, the LCDR1 sequence comprises SEQ ID NO: 46, the LCDR2 sequence comprises YAS, and the LCDR3 sequence comprises SEQ ID NO: 66, or
the HCDR1 sequence comprises SEQ ID NO: 11, the HCDR2 sequence comprises SEQ ID NO: 23, the HCDR3 sequence comprises SEQ ID NO: 36, the LCDR1 sequence comprises SEQ ID NO: 47, the LCDR2 sequence comprises STS, and the LCDR3 sequence comprises SEQ ID NO: 67.

Aspect 63: The method of aspect 61 or 62, wherein the VH domain comprises at least 80%, at least 85%, at least 90%, at least 95%, at least 97%, at least 99%, or 100% sequence identity to a sequence selected from SEQ ID NOs: 68-80.

Aspect 64: The method of any one of aspects 61-63, wherein the VL domain comprises at least 80%, at least 85%, at least 90%, at least 95%, at least 97%, at least 99%, or 100% identity to a sequence selected from SEQ ID NOs: 81-93.

Aspect 65: The method of any one of aspects 61-64, wherein the VH domain comprises a portion of a heavy chain comprising at least 80%, at least 85%, at least 90%, at least 95% sequence identity to a sequence selected from SEQ ID NOs: 94-101.

Aspect 66: The method of any one of aspects 61-65, wherein the VL domain comprises a portion of a light chain comprising at least 80%, at least 85%, at least 90%, at least 95% sequence identity to a sequence selected from SEQ ID NOs: 102-105.

Aspect 67: The method of any one of aspects 61-66, wherein the anti-CD122 antibody is a monoclonal antibody.

Aspect 68: The method of any one of aspects 61-67, wherein the anti-CD122 antibody is a humanized antibody.

Aspect 69: The method of any one of aspects 61-68, wherein the anti-CD122 antibody or its antigen-binding fragment thereof comprises IgG-scFv, IgA, IgM, IgE antibody, mini-antibody, minibody, scFv-CH3 KIH, Fab-scFv-Fc KIH, Fab-scFv, scFv-CH-CL-scFv, Fab′, F(ab′)2, F(ab′)3, F(ab′)2-scFv2, scFv, scFv-KIH, Fab-scFv-Fc, or intrabody.

Aspect 70: The method of any one of aspects 61-69, wherein the anti-CD122 antibody interferes with IL-15 binding to the intermediate affinity IL-βγ receptor composed of CD122 beta chain and CD132 gamma chain subunits.

Aspect 71: The method of any one of aspects 61-70, wherein the anti-CD122 antibody diminishes or disrupts IL-15-induced signal transduction.

Aspect 72: The method of any one of aspects 61-71, wherein the anti-CD122 antibody interferes with IL-2 binding to the intermediate affinity IL-βγ receptor composed of CD122 beta chain and CD132 gamma chain subunits.

Aspect 73: The method of any one of aspects 61-72, wherein the anti-CD122 antibody diminishes or disrupts IL-2-induced signal transduction mediated through the intermediate affinity IL-βγ receptor composed of CD122 beta chain and CD132 gamma chain subunits.

Aspect 74: The method of any one of aspects 61-73, wherein the GvHD is acute graft versus host disease (aGvHD).

Aspect 75: The method of any one of aspects 61-74, wherein the administering an effective amount of the anti-CD122 antibody delays an onset of one or more symptoms of aGvHD in the subject.

Aspect 76: The method of any one of aspects 61-75, wherein the administering an effective amount of the anti-CD122 antibody alleviates one or more symptoms of aGvHD in the subject.

Aspect 77: The method of aspects 75 or 76, wherein the one or more symptoms of aGvHD is selected from a group consisting of: itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, and weight loss.

Aspect 78: The method of any one of aspects 61-73, wherein the GvHD is chronic graft versus host disease (cGvHD).

Aspect 79: The method of aspect 78, wherein the administering an effective amount of the anti-CD122 antibody delays an onset of one or more symptoms of chronic GvHD (cGvHD) in the subject.

Aspect 80: The method of aspect 78 or 79, wherein the administering an effective amount of the anti-CD122 antibody alleviates one or more symptoms of cGvHD in the subject.

Aspect 81: The method of aspect 79 or 80, wherein the one or more symptoms of cGvHD is selected from a group consisting of: skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, and changes in hair texture.

Aspect 82: The method of any one of aspects 61-81, wherein the administering an effective amount of the anti-CD122 antibody increases the survival rate of the subject.

Aspect 83: The method of any one of aspects 61-82, wherein the anti-CD122 antibody is administered systemically, locally, intradermally, subcutaneously, or topically.

Aspect 84: The method of aspect 83, wherein the anti-CD122 antibody administered systemically is administered by intravenous injection.

Aspect 85: The method of any one of aspects 61-84, wherein the method further comprises administering to the subject an effective amount of a JAK inhibitor, thereby preventing or treating GvHD in the subject.

Aspect 86: The method of aspect 85, wherein the anti-CD122 antibody and the JAK inhibitor are co-administered.

Aspect 87: The method of aspect 85 or 86, wherein the anti-CD122 antibody and the JAK inhibitor are administered separately or sequentially.

Aspect 88: The method of any one of aspects 85-87, wherein the JAK inhibitor is selected from ruxolitinib, abrocitinib, baricitinib, delgocitinib, fedratinib, filgotinib, oclacitinib, pacritinib, peficitinib, tofacitinib, itacitinib and upadacitinib.

Aspect 89: The method of any one of aspects 85-88, wherein the JAK inhibitor is ruxolitinib.

Aspect 90: The method of any one of aspects 85-89, wherein the JAK inhibitor is administered first and the anti-CD122 antibody is administered second.

Aspect 91: The method of any one of aspects 85-90, wherein the administering an effective amount of the anti-CD122 antibody and the JAK inhibitor delays an onset of one or more symptoms of aGvHD or cGvHD in the subject.

Aspect 92: The method of any one of aspects 85-91, wherein the administering an effective amount of the anti-CD122 antibody and the JAK inhibitor alleviates one or more symptoms of aGvHD or cGvHD in the subject.

Aspect 93: The method of any one of aspects 85-92, wherein the administering an effective amount of the anti-CD122 antibody and the JAK inhibitor increases the survival rate of the subject.

Aspect 94: The method of any one of aspects 85-93, wherein the administering an effective amount of the anti-CD122 antibody and the JAK inhibitor increases the survival rate of the subject compared with a subject treated with a JAK inhibitor as a monotherapy

Aspect 95: The method of any one of aspects 85-94, wherein the anti-CD122 antibody and the JAK inhibitor are administered by the same route of administration.

Aspect 96: The method of any one of aspects 85-95, wherein the anti-CD122 antibody and the JAK inhibitor are administered by separate routes of administration.

Aspect 97: The method of any one of aspects 85-96, wherein the anti-CD122 antibody and the JAK inhibitor are administered by separate routes of administration.

Aspect 98: The method of any one of aspects 85-97, wherein the anti-CD122 antibody is administered systemically, locally, intradermally, subcutaneously, or topically, and wherein the JAK inhibitor is administered systemically, locally, intradermally, subcutaneously, or topically

Aspect 99: The method of aspect 98, wherein the anti-CD122 antibody administered systemically is administered by intravenous injection or intraperitoneal injection.

Aspect 100: The method of aspect 98 or 99, wherein the JAK inhibitor administered systemically is administered by intravenous injection, by enteral administration, or through inhalation.

Aspect 101: The method of aspect 100, wherein the JAK inhibitor administered by enteral administration is administered orally.

What is claimed is:

1. A method of preventing or treating graft versus host disease (GvHD) in a subject in need thereof, the method comprising:
administering to the subject an effective amount of an anti-CD122 antibody, or its antigen-binding fragment thereof, comprising i) a variable heavy chain (VH) domain and ii) a variable light chain (VL) domain, wherein the VH domain comprises an HCDR1 sequence comprising a sequence selected from SEQ ID NOs: 1-11, an HCDR2 sequence comprising a sequence selected from SEQ ID NOs: 12-23, and an HCDR3 sequence comprising a sequence selected from SEQ ID NOs: 24-36, and the VL domain comprises an LCDR1 sequence comprising a sequence selected from SEQ ID NOs: 37-47, an LCDR2 sequence comprising a sequence selected from GTS, TTS, YTS, WAS, KAS, GAT, YAS or STS, and an LCDR3 sequence comprising a sequence selected from SEQ ID NOs: 56-67.

2. The method of claim 1, wherein:
i. the HCDR1 sequence comprises SEQ ID NO: 7, the HCDR2 sequence comprises SEQ ID NO: 18, the HCDR3 sequence comprises SEQ ID NO: 30, the LCDR1 sequence comprises SEQ ID NO: 43, the LCDR2 sequence comprises YTS, and the LCDR3 sequence comprises SEQ ID NO: 62,
ii. the HCDR1 sequence comprises SEQ ID NO: 1, the HCDR2 sequence comprises SEQ ID NO: 12, the HCDR3 sequence comprises SEQ ID NO: 24, the LCDR1 sequence comprises SEQ ID NO: 37, the LCDR2 sequence comprises GTS, and the LCDR3 sequence comprises SEQ ID NO: 56,
iii. the HCDR1 sequence comprises SEQ ID NO: 2, the HCDR2 sequence comprises SEQ ID NO: 13, the HCDR3 sequence comprises SEQ ID NO: 25, the LCDR1 sequence comprises SEQ ID NO: 38, the LCDR2 sequence comprises TTS, and the LCDR3 sequence comprises SEQ ID NO: 57,
iv. the HCDR1 sequence comprises SEQ ID NO: 3, the HCDR2 sequence comprises SEQ ID NO: 14, the HCDR3 sequence comprises SEQ ID NO: 26, the LCDR1 sequence comprises SEQ ID NO: 39, the LCDR2 sequence comprises YTS, and the LCDR3 sequence comprises SEQ ID NO: 58,
v. the HCDR1 sequence comprises SEQ ID NO: 4, the HCDR2 sequence comprises SEQ ID NO: 15, the HCDR3 sequence comprises SEQ ID NO: 27, the LCDR1 sequence comprises SEQ ID NO: 40, the LCDR2 sequence comprises WAS, and the LCDR3 sequence comprises SEQ ID NO: 59,
vi. the HCDR1 sequence comprises SEQ ID NO: 5, the HCDR2 sequence comprises SEQ ID NO: 16, the HCDR3 sequence comprises SEQ ID NO: 28, the LCDR1 sequence comprises SEQ ID NO: 41, the LCDR2 sequence comprises YTS, and the LCDR3 sequence comprises SEQ ID NO: 60,
vii. the HCDR1 sequence comprises SEQ ID NO: 6, the HCDR2 sequence comprises SEQ ID NO: 17, the HCDR3 sequence comprises SEQ ID NO: 29, the LCDR1 sequence comprises SEQ ID NO: 42, the LCDR2 sequence comprises KAS, and the LCDR3 sequence comprises SEQ ID NO: 61,
viii. the HCDR1 sequence comprises SEQ ID NO: 8, the HCDR2 sequence comprises SEQ ID NO: 19, the HCDR3 sequence comprises SEQ ID NO: 31, the LCDR1 sequence comprises SEQ ID NO: 44, the LCDR2 sequence comprises YTS, and the LCDR3 sequence comprises SEQ ID NO: 63,
ix. the HCDR1 sequence comprises SEQ ID NO: 9, the HCDR2 sequence comprises SEQ ID NO: 20, the HCDR3 sequence comprises SEQ ID NO: 32, the LCDR1 sequence comprises SEQ ID NO: 45, the LCDR2 sequence comprises GAT, and the LCDR3 sequence comprises SEQ ID NO: 64,
x. the HCDR1 sequence comprises SEQ ID NO: 1, the HCDR2 sequence comprises SEQ ID NO: 21, the HCDR3 sequence comprises SEQ ID NO: 33, the LCDR1 sequence comprises SEQ ID NO: 37, the LCDR2 sequence comprises GTS, and the LCDR3 sequence comprises SEQ ID NO: 65,
xi. the HCDR1 sequence comprises SEQ ID NO: 1, the HCDR2 sequence comprises SEQ ID NO: 21, the HCDR3 sequence comprises SEQ ID NO: 34, the LCDR1 sequence comprises SEQ ID NO: 37, the LCDR2 sequence comprises GTS, and the LCDR3 sequence comprises SEQ ID NO: 65,
xii. the HCDR1 sequence comprises SEQ ID NO: 10, the HCDR2 sequence comprises SEQ ID NO: 22, the HCDR3 sequence comprises SEQ ID NO: 35, the LCDR1 sequence comprises SEQ ID NO: 46, the LCDR2 sequence comprises YAS, and the LCDR3 sequence comprises SEQ ID NO: 66, or
xiii. the HCDR1 sequence comprises SEQ ID NO: 11, the HCDR2 sequence comprises SEQ ID NO: 23, the HCDR3 sequence comprises SEQ ID NO: 36, the LCDR1 sequence comprises SEQ ID NO: 47, the LCDR2 sequence comprises STS, and the LCDR3 sequence comprises SEQ ID NO: 67.

3. The method of claim 1, wherein the VH domain comprises a portion of a heavy chain comprising at least 85% sequence identity to a sequence selected from SEQ ID NOs: 94-101.

4. The method of claim 1, wherein the VL domain comprises a portion of a light chain comprising at least 85% sequence identity to a sequence selected from SEQ ID NOs: 102-105.

5. The method of claim 1, wherein the anti-CD122 antibody is a humanized monoclonal antibody.

6. The method of claim 1, wherein the anti-CD122 antibody or its antigen-binding fragment thereof comprises IgG-scFv, IgA, IgM, IgE antibody, mini-antibody, minibody, scFv-CH3 KIH, Fab-scFv-Fc KIH, Fab-scFv, scFv-CH-CL-scFv, Fab′, F(ab′)2, F(ab′)3,_F(ab′)2-scFv2, scFv, scFv-KIH, Fab-scFv-Fc, or intrabody.

7. The method of claim 1, wherein the anti-CD122 antibody interferes with Interleukin-15 (IL-15) binding to the intermediate affinity IL-βγ receptor composed of CD122 beta chain and CD132 gamma chain subunits, and wherein the anti-CD122 antibody does not significantly disrupt an Interleukin-2 (IL-2) from:
a) binding to a high affinity IL2Rα/IL2Rβ/IL2Rγ complex in cells of the subject, or
b) signaling through a high affinity IL2Rα/IL2Rβ/IL2Rγ complex in cells of the subject.

8. The method of claim 1, wherein the anti-CD122 antibody diminishes or disrupts:
a) an IL-2 from binding to an IL2Rβ/IL2Rγ complex,
b) an IL-2-induced signal transduction mediated through the intermediate affinity IL-βγ receptor composed of CD122 beta chain and CD132 gamma chain subunits,
c) an IL-15/IL15Ra complex from binding to an IL15Rβ/IL15Rγ complex,
d) an IL-15-induced signal transduction,
e) a CD122-mediated signal transduction, or
f) any combination of a) through e).

9. The method of claim 1, wherein the GvHD is acute graft versus host disease (aGvHD), wherein the administering an effective amount of the anti-CD122 antibody:
a) delays an onset of one or more symptoms of aGvHD in the subject,
b) alleviates one or more symptoms of aGvHD in the subject, or
c) both a) and b); and
wherein the one or more symptoms of aGvHD is selected from a group consisting of: itchy skin, skin rash, reddened patches on the skin, yellow discoloration of the skin, blisters on the skin, exposed surfaces of the skin flaking off, yellow discoloration of the eyes, jaundice, elevated liver enzyme levels in the blood, nausea, vomiting, diarrhea, abdominal cramping, loss of appetite, and weight loss.

10. The method of claim 1, wherein the GvHD is chronic graft versus host disease (cGvHD), wherein the administering an effective amount of the anti-CD122 antibody:
a) delays an onset of one or more symptoms of chronic GvHD (cGvHD) in the subject,
b) alleviates one or more symptoms of cGvHD in the subject, or
c) both a) and b); and
wherein the one or more symptoms of cGvHD is selected from a group consisting of: skin rash, raised skin, discolored skin, itchy skin, thickened skin, tightened skin, damaged sweat glands, intolerance to temperature changes, abdominal swelling, yellow discoloration of the eyes, jaundice, elevated or abnormal liver enzyme levels in the blood, dry eyes, changes in vision, dry mouth, white patches in the oral cavity, painful mouth ulcers, pain or sensitivity to hot, cold, spicy, and/or acidic foods, pain or sensitivity to carbonated beverages, shortness of breath, dry cough, chronic cough, wheezing, difficulty breathing, pulmonary changes observed on a chest X-ray, difficulty swallowing, difficulty eating, pain with swallowing, gum disease, tooth decay, loss of appetite, weight loss, nausea, vomiting, diarrhea, stomach pain, fatigue, muscle weakness, muscle cramps, neuromuscular pain, decreased range of motion in joints, decreased range of extension of fingers, wrists, elbows, knees, and/or ankles, tightness in joints or in connective tissue, change in physical activity level, change in locomotor activity level, change in posture, change in gait, vaginal dryness, vaginal itching, vaginal pain, vaginal ulcerations and scarring, narrowing of the vagina, painful vaginal intercourse, narrowing and/or scaring of the urethra, itching and/or scarring of the penis and scrotum, irritation of the penis, change in skin texture, change in skin integrity, scaling of skin, areas of denuded skin, loss of hair on the head, hard nails, brittle nails, nail loss, changes in nail texture, premature graying of the hair, and changes in hair texture.

11. The method of claim 1, wherein the administering an effective amount of the anti-CD122 antibody increases the survival rate of the subject.

12. The method of claim 1, wherein the anti-CD122 antibody is administered systemically, locally, intradermally, subcutaneously, or topically.

13. The method of claim 12, wherein the anti-CD122 antibody administered systemically is administered by intravenous injection.

14. The method of claim 1, wherein the method further comprises administering to the subject an effective amount of a JAK inhibitor, thereby preventing or treating GvHD in the subject.

15. The method of claim 14, wherein the JAK inhibitor is selected from the group consisting of ruxolitinib, abrocitinib, baricitinib, delgocitinib, fedratinib, filgotinib, oclacitinib, pacritinib, peficitinib, tofacitinib, itacitinib and upadacitinib.

16. The method of claim 14, wherein the administering an effective amount of the anti-CD122 antibody and the JAK inhibitor:
a) delays an onset of one or more symptoms of aGvHD or cGvHD in the subject,
b) alleviates one or more symptoms of aGvHD or cGvHD in the subject, or
c) both a) and b).

17. The method of claim 14, wherein the administering an effective amount of the anti-CD122 antibody and the JAK inhibitor increases the survival rate of the subject compared with a subject treated with a JAK inhibitor as a monotherapy.

18. The method of claim 14, wherein the anti-CD122 antibody is administered systemically, locally, intradermally, subcutaneously, or topically, and wherein the JAK inhibitor is administered systemically, locally, intradermally, subcutaneously, or topically.

19. The method of claim 18, wherein the anti-CD122 antibody administered systemically is administered by intravenous injection or intraperitoneal injection.

20. The method of claim 18, wherein the JAK inhibitor administered systemically is administered by intravenous injection, by enteral administration, or through inhalation.


SEQUENCE LISTING (253 sequences)

SEQ ID NO: 1 - protein, 8 aa, synthetic construct
GFSLTSYG

SEQ ID NO: 2 - protein, 8 aa, synthetic construct
GYTFTSYW

SEQ ID NO: 3 - protein, 8 aa, synthetic construct
GSTFNRYW

SEQ ID NO: 4 - protein, 8 aa, synthetic construct
GFTFTDYN

SEQ ID NO: 5 - protein, 8 aa, synthetic construct
GYSFTAYT

SEQ ID NO: 6 - protein, 8 aa, synthetic construct
GFNIKDDY

SEQ ID NO: 7 - protein, 8 aa, synthetic construct
GYTFTSHW

SEQ ID NO: 8 - protein, 8 aa, synthetic construct
GFTFSTFA

SEQ ID NO: 9 - protein, 8 aa, synthetic construct
GFTFTDHT

SEQ ID NO: 10 - protein, 8 aa, synthetic construct
GFSLTSYD

SEQ ID NO: 11 - protein, 8 aa, synthetic construct
GYTFTAYW

SEQ ID NO: 12 - protein, 7 aa, synthetic construct
MWGGGST

SEQ ID NO: 13 - protein, 8 aa, synthetic construct
IYPGRGST

SEQ ID NO: 14 - protein, 8 aa, synthetic construct
ILPGSGNT

SEQ ID NO: 15 - protein, 8 aa, synthetic construct
INPNNGRS

SEQ ID NO: 16 - protein, 8 aa, synthetic construct
INPYNGYA

SEQ ID NO: 17 - protein, 8 aa, synthetic construct
IDPENGDT

SEQ ID NO: 18 - protein, 8 aa, synthetic construct
IYPGSGNT

SEQ ID NO: 19 - protein, 9 aa, synthetic construct
ITGDGGTYT

SEQ ID NO: 20 - protein, 8 aa, synthetic construct
IYPRDGYT

SEQ ID NO: 21 - protein, 8 aa, synthetic construct
IWGGGGST

SEQ ID NO: 22 - protein, 7 aa, synthetic construct
IWTGGGT

SEQ ID NO: 23 - protein, 8 aa, synthetic construct
IDPNSGYT

SEQ ID NO: 24 - protein, 15 aa, synthetic construct
ARRTYSDSYYYEMDY

SEQ ID NO: 25 - protein, 9 aa, synthetic construct
ARELGGFAY

SEQ ID NO: 26 - protein, 14 aa, synthetic construct
ARLDYYGSRYYFDY

SEQ ID NO: 27 - protein, 13 aa, synthetic construct
AREDWEGFYAMDY

SEQ ID NO: 28 - protein, 9 aa, synthetic construct
ARVGYYFDY

SEQ ID NO: 29 - protein, 6 aa, synthetic construct
TGYFDY

SEQ ID NO: 30 - protein, 9 aa, synthetic construct
ARERGGFDY

SEQ ID NO: 31 - protein, 11 aa, synthetic construct
ARHSVSSWFAY

SEQ ID NO: 32 - protein, 11 aa, synthetic construct
ARPTSLLRFPY

SEQ ID NO: 33 - protein, 14 aa, synthetic construct
ARHNYDGYYYSLDY

SEQ ID NO: 34 - protein, 14 aa, synthetic construct
ARHNYDNYYYTLDY

SEQ ID NO: 35 - protein, 11 aa, synthetic construct
VRDLFPYAMDY

SEQ ID NO: 36 - protein, 10 aa, synthetic construct
ARGHFGYDDS

SEQ ID NO: 37 - protein, 7 aa, synthetic construct
SSVSSRY

SEQ ID NO: 38 - protein, 7 aa, synthetic construct
SYVSSSY

SEQ ID NO: 39 - protein, 6 aa, synthetic construct
QDIRNY

SEQ ID NO: 40 - protein, 6 aa, synthetic construct
QNVDTD

SEQ ID NO: 41 - protein, 5 aa, synthetic construct
SSVSY

SEQ ID NO: 42 - protein, 6 aa, synthetic construct
QNINLW

SEQ ID NO: 43 - protein, 6 aa, synthetic construct
QDISNF

SEQ ID NO: 44 - protein, 5 aa, synthetic construct
SSVNY

SEQ ID NO: 45 - protein, 6 aa, synthetic construct
EDIDSY

SEQ ID NO: 46 - protein, 6 aa, synthetic construct
QTIGTS

SEQ ID NO: 47 - protein, 5 aa, synthetic construct
SRVSY

SEQ ID NO: 48-55 - skipped (8 entries)

SEQ ID NO: 56 - protein, 9 aa, synthetic construct
QQYHSDPLT

SEQ ID NO: 57 - protein, 9 aa, synthetic construct
HQYHLSPWT

SEQ ID NO: 58 - protein, 9 aa, synthetic construct
QQGDPLPPT

SEQ ID NO: 59 - protein, 9 aa, synthetic construct
EQYSSYPYT

SEQ ID NO: 60 - protein, 9 aa, synthetic construct
QQFTSSPYT

SEQ ID NO: 61 - protein, 9 aa, synthetic construct
LQGQSYPWT

SEQ ID NO: 62 - protein, 9 aa, synthetic construct
QQDNNHPYT

SEQ ID NO: 63 - protein, 9 aa, synthetic construct
QQFTSSPWT

SEQ ID NO: 64 - protein, 9 aa, synthetic construct
QHYYSIPYT

SEQ ID NO: 65 - protein, 9 aa, synthetic construct
QQYHGDPLT

SEQ ID NO: 66 - protein, 9 aa, synthetic construct
QQTNSWPLT

SEQ ID NO: 67 - protein, 9 aa, synthetic construct
QQRSSYPLT

SEQ ID NO: 68 - protein, 121 aa, synthetic construct
QVQLKESGPGLVAPSQSLSITCTVSGFSLTSYGIDWVRQPPGKGLEWLGIMWGGGSTNYNSALMSRLSISKDISKSQVFLKVNSLQTDDTAMYYCARRTYSDSYYYEMDYWGQGTSVTVSS

SEQ ID NO: 69 - protein, 116 aa, synthetic construct
QVQLQQPGTELVKPGASVKMSCKASGYTFTSYWITWVKQRPGQGLEWIGDIYPGRGSTNYNEKFKSKATLTVDTSSSTAYMQLSSLTSEDSAVYYCARELGGFAYWGQGTLVTVSA

SEQ ID NO: 70 - protein, 121 aa, synthetic construct
QVQLQQSGAELMKPGASVKISCKATGSTFNRYWIEWVKQRPGHGLEWIGEILPGSGNTNYNEKFKGKATFTADTSSNTAYMQLSSLTSEDSAVYYCARLDYYGSRYYFDYWGQGTTLTVSS

SEQ ID NO: 71 - protein, 120 aa, synthetic construct
EVQLQQSGPELVKPGASVKMSCKASGFTFTDYNIHWVKQSHGKSLEWIGYINPNNGRSSYNLYFKDKATLTVNKSSSTAYMELRSLTSEDSAVYYCAREDWEGFYAMDYWGQGTSVTVSS

SEQ ID NO: 72 - protein, 116 aa, synthetic construct
EVQLQQSGPELVKPGASMKISCKASGYSFTAYTMNWVRQSHGKNLEWIGLINPYNGYANYNQKFKGKATLTVDKSSSTAYMDLLSLTSEDSAVYYCARVGYYFDYWGQGTTLTVSS

SEQ ID NO: 73 - protein, 113 aa, synthetic construct
EVQLQQSGAELVRPGASVKLSCTSSGFNIKDDYMHWVKQRPEQGLEWIGWIDPENGDTEYASKFQGKATITADTSSNTAYLQLNSLTSEDTAVYYCTGYFDYWGQGTTLTVSS

SEQ ID NO: 74 - protein, 116 aa, synthetic construct
QVQLQQPGAELVKPGASVKMSCKASGYTFTSHWITWVKQRPGQGLEWIGDIYPGSGNTNYNEKFKSKATLTVDTSSSTAYMQLSSLTSEDSADYYCARERGGFDYWGQGTTLTVSS

SEQ ID NO: 75 - protein, 119 aa, synthetic construct
DVILVESGGDLVKPGGSLKLSCAASGFTFSTFAMSWVRQTPEKRLEWVASITGDGGTYTYYSDSVKGRFTISRDNARNTLYLQMSSLRSEDTAFYYCARHSVSSWFAYWGQGTLVTVSA

SEQ ID NO: 76 - protein, 118 aa, synthetic construct
QVQLLQSDAELVKPGASVKISCKVSGFTFTDHTLHWMKQRPEQGLEWIGYIYPRDGYTKYNEKFKGKATLTADKSSSTAYMQLDSLTSEDSAVYFCARPTSLLRFPYWGQGTLVTVSA

SEQ ID NO: 77 - protein, 121 aa, synthetic construct
QVQLKESGPGLVAPSQSLSITCTVSGFSLTSYGVDWVRQPPGKGLEWLGVIWGGGGSTNYNSALMSRLSISKDNSKSQVFLKMNSLQTDDTAMYYCARHNYDGYYYSLDYWGQGTSVTVSS

SEQ ID NO: 78 - protein, 121 aa, synthetic construct
QVQLKESGPGLVAPSQSLSITCTVSGFSLTSYGVDWVRQPPGKGLEWLGVIWGGGGSTNYNSALMSRLSISKDNSKSQVFLKMNSLQTDDTAMYYCARHNYDNYYYTLDYWGQGTSVTVSS

SEQ ID NO: 79 - protein, 117 aa, synthetic construct
QVQLKESGPGLVAPSQSLSITCTVSGFSLTSYDISWIRQPPGKGLEWLGVIWTGGGTNYNSPFMSRLTISKDNSRSQVFLKMNSLQTDDTAMYYCVRDLFPYAMDYWGQGTSVTVSS

SEQ ID NO: 80 - protein, 117 aa, synthetic construct
QVQLQQSGAELAKPGASVKMSCKASGYTFTAYWIHWVKQRPGQGLEWIGYIDPNSGYTDYNQIFKDKATLTADRSSSTAYMQLNSLTSEDSAVYYCARGHFGYDDSWGQGITLTVSS

SEQ ID NO: 81 - protein, 108 aa, synthetic construct
QIVLTQSPAIMSASPGEKVTMTCSASSSVSSRYLHWYQQKSGASPKLWIYGTSNLASGVPARFSGSGSGTSYSLTISSVEAEDAATYYCQQYHSDPLTFGAGTKLELK

SEQ ID NO: 82 - protein, 108 aa, synthetic construct
QIVLTQSPAIMSASLGERVTMTCTASSYVSSSYLHWYQQKPGSSPTLWIYTTSNLASGVPARFSGSGSGTSYSLTISNMEAEDAATYYCHQYHLSPWTFGGGTKLEIK

SEQ ID NO: 83 - protein, 107 aa, synthetic construct
DIQMTQTTSSLSASLGDRVTISCRASQDIRNYLNWYQQKPDGTLKLLIYYTSRLHSGVPSRFSGSGSGTDYSLTISNLEQEDVATYFCQQGDPLPPTFGGGTKVEIK

SEQ ID NO: 84 - protein, 107 aa, synthetic construct
DIVMTQSQKFMSTSVGDRVSITCKASQNVDTDVSWYQQKPGKSPKTLIYWASNRFTGVPDRFTGSGSGTDFTLTITNVQSEDLADYFCEQYSSYPYTFGSGTKLEIK

SEQ ID NO: 85 - protein, 106 aa, synthetic construct
ENVLTQSPTIMSASLGEKVTMSCRASSSVSYMHWYQQKSDASPKLWIYYTSNLAPGVPARFSASGSGNSYSLTISSMEGEDAATYYCQQFTSSPYTFGGGTKLEIK

SEQ ID NO: 86 - protein, 107 aa, synthetic construct
DIQMNQSPSSLSASLGDTISISCRASQNINLWLNWYQQKPGNVPKLLIFKASNLHPGVPSRFSGSGSGTDFTLTISSLQPEDIATYYCLQGQSYPWTFGGGTKLEIK

SEQ ID NO: 87 - protein, 107 aa, synthetic construct
DIQMTQTTSSLSASLGDRVTINCRASQDISNFLNWYQQKPDGTLKLLIYYTSGLHSGVPSRFSGSGSGTDYSLTISNLEEEDIATYFCQQDNNHPYTFGSGTKLEIK

SEQ ID NO: 88 - protein, 106 aa, synthetic construct
ENVLTQSPAIMSASLGEKVTMSCRASSSVNYMYWYQQKSDASPKLWIYYTSNLAPGVPARFSGSGSGNSYSLTISSMEGEDAATYYCQQFTSSPWTFGGGTKLEIK

SEQ ID NO: 89 - protein, 107 aa, synthetic construct
DIQMTQSPASLSASVGETVTITCRASEDIDSYLAWYQQKQGKSPQLLVYGATLLADGVPSRFSGSGSGTQYSLKINSLQSEDVARYYCQHYYSIPYTFGSGTKLEIK

SEQ ID NO: 90 - protein, 108 aa, synthetic construct
QIVLTQSPAIMSASPGEKVTMTCSASSSVSSRYLHWYQQKSGASPKLWIYGTSNLASGVPARFSGSGSGTSYSLTISSVEAEDAATYYCQQYHGDPLTFGAGTKLELK

SEQ ID NO: 91 - protein, 108 aa, synthetic construct
QIVLTQSPAIMSASPGEKVTMTCSASSSVSSRYLHWYQQKSGASPKLWIYGTSNLASGVPARFSGSGSGTSYSLTISSVEAEDAASYYCQQYHGDPLTFGAGTKLELK

SEQ ID NO: 92 - protein, 107 aa, synthetic construct
DILLTQSPAILSVSPGERVSLSCRASQTIGTSIHWYQQRTNGSIRLLIKYASESISGIPSRFSGSGFGTDFALSINSVESEDIADYFCQQTNSWPLTFGAGTKLELK

SEQ ID NO: 93 - protein, 106 aa, synthetic construct
QIVLTQSPAIMSASPGEKVTITCSASSRVSYMHWFQQKPGTSPKLWIYSTSNLASGVPARFSGSGSGTSYSLTISRMEAEDAATYYCQQRSSYPLTFGAGTKLELK

SEQ ID NO: 94 - protein, 485 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLQQPGAELVKPGASVKMSCKASGYTFTSHWITWVKQRPGQGLEWIGDIYPGSGNTNYNEKFKSKATLTVDTSSSTAYMQLSSLTSEDSADYYCARERGGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 485 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLVQSGAEVKKPGASVKVSCKASGYTFTSHWISWVRQAPGQGLEWMGDIYPGSGNTNYNEKLQGRVTMTVDTSTSTAYMELRSLRSDDTAVYYCARERGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 485 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLVQSGAEVKKPGASVKVSCKASGYTFTSHWITWVRQAPGQGLEWMGDIYPGSGNTNYNEKFQGRVTLTVDTSISTAYMELSRLRSDDTVVYYCARERGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 485 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLVQSGAEVKKPGASVKVSCKASGYTFTSHWITWVRQAPGQRLEWMGDIYPGSGNTNYNEKFQGRVTITVDTSASTAYMELSSLRSEDTAVYYCARERGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 482 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLQQPGAELVKPGASVKMSCKASGYTFTSHWITWVKQRPGQGLEWIGDIYPGSGNTNYNEKFKSKATLTVDTSSSTAYMQLSSLTSEDSADYYCARERGGFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 99 - protein, 482 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLVQSGAEVKKPGASVKVSCKASGYTFTSHWISWVRQAPGQGLEWMGDIYPGSGNTNYNEKLQGRVTMTVDTSTSTAYMELRSLRSDDTAVYYCARERGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 482 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLVQSGAEVKKPGASVKVSCKASGYTFTSHWITWVRQAPGQGLEWMGDIYPGSGNTNYNEKFQGRVTLTVDTSISTAYMELSRLRSDDTVVYYCARERGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 101 - protein, 482 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXQVQLVQSGAEVKKPGASVKVSCKASGYTFTSHWITWVRQAPGQRLEWMGDIYPGSGNTNYNEKFQGRVTITVDTSASTAYMELSSLRSEDTAVYYCARERGGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 102 - protein, 254 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXDIQMTQTTSSLSASLGDRVTINCRASQDISNFLNWYQQKPDGTLKLLIYYTSGLHSGVPSRFSGSGSGTDYSLTISNLEEEDIATYFCQQDNNHPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 254 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXDIQMTQSPSSLSASVGDRVTITCRASQDISNFLNWYQQKPEGTLKLLIYYTSGLHSGVPSRFSGSGSGTDFTLTISSLQPEDVATYYCQQDNNHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 254 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXDIQMTQSPSSLSASVGDRVTITCQASQDISNFLNWYQQKPGKTLKLLIYYTSGLHTGVPSRFSGSGSGTDFTFTISSLQPEDIATYYCQQDNNHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 254 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXDIQMTQSPSSLSASVGDRVTITCRASQDISNFLNWYQQKPGKAPKLLIYYTSGLHSGVPSRFSGSGSGTDFTLTISSLQPEDFATYYCQQDNNHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 24 aa, synthetic construct
MDPKGSLSWRILLFLSLAFELSYG

SEQ ID NO: 107 - protein, 20 aa, synthetic construct
METDTLLLWVLLLWVPGSTG

SEQ ID NO: 108 - protein, 19 aa, synthetic construct
MGWSLILLFLVAVATRVHS

SEQ ID NO: 109 - protein, 20 aa, synthetic construct
MRVPAQLLGLLLLWLPGARC

SEQ ID NO: 110 - protein, 5 aa, synthetic construct
SYGVH

SEQ ID NO: 111 - protein, 5 aa, synthetic construct
SYAVH

SEQ ID NO: 112 - protein, 5 aa, synthetic construct
SYAMH

SEQ ID NO: 113 - protein, 5 aa, synthetic construct
SYGMH

SEQ ID NO: 114 - protein, 5 aa, synthetic construct
DFYME

SEQ ID NO: 115 - protein, 7 aa, synthetic construct
NDNHWWN

SEQ ID NO: 116 - protein, 16 aa, synthetic construct
VIWSGGSTDYNAAFIS

SEQ ID NO: 117 - protein, 16 aa, synthetic construct
AIWSGGSTDYNAAVKG

SEQ ID NO: 118 - protein, 16 aa, synthetic construct
AIWSGGSTDYAASVKG

SEQ ID NO: 119 - protein, 16 aa, synthetic construct
AIWSGGSTDYADAVKG

SEQ ID NO: 120 - protein, 16 aa, synthetic construct
VIWSGGSTDYADSVKG

SEQ ID NO: 121 - protein, 16 aa, synthetic construct
VIWSGGSTDYADAVKG

SEQ ID NO: 122 - protein, 19 aa, synthetic construct
ASRNKANDYTTEYSASVKG

SEQ ID NO: 123 - protein, 16 aa, synthetic construct
YIDSSGSSDNNPSLKS

SEQ ID NO: 124 - protein, 11 aa, synthetic construct
AGDYNYDGFAY

SEQ ID NO: 125 - protein, 11 aa, synthetic construct
AGDANYDGFAY

SEQ ID NO: 126 - protein, 11 aa, synthetic construct
AGDYEYDGFAY

SEQ ID NO: 127 - protein, 11 aa, synthetic construct
AGDKNYDGFAY

SEQ ID NO: 128 - protein, 11 aa, synthetic construct
AGDQNYDGFAY

SEQ ID NO: 129 - protein, 10 aa, synthetic construct
SYYRYDGMDY

SEQ ID NO: 130 - protein, 11 aa, synthetic construct
GGGRDYYGMDY

SEQ ID NO: 131 - protein, 10 aa, synthetic construct
SGSSSVSFMY

SEQ ID NO: 132 - protein, 10 aa, synthetic construct
QASQSVSFLY

SEQ ID NO: 133 - protein, 10 aa, synthetic construct
QASSSVSFMY

SEQ ID NO: 134 - protein, 10 aa, synthetic construct
QASQDISFMY

SEQ ID NO: 135 - protein, 10 aa, synthetic construct
QASQSVSHLY

SEQ ID NO: 136 - protein, 10 aa, synthetic construct
QASQSVSYMY

SEQ ID NO: 137 - protein, 10 aa, synthetic construct
QASSSISYMY

SEQ ID NO: 138 - protein, 10 aa, synthetic construct
QASSSVSHLY

SEQ ID NO: 139 - protein, 10 aa, synthetic construct
SAISSVSYMY

SEQ ID NO: 140 - protein, 15 aa, synthetic construct
RASQSVSTSSYSYVH

SEQ ID NO: 141 - protein, 7 aa, synthetic construct
DTSNLAS

SEQ ID NO: 142 - protein, 7 aa, synthetic construct
DTSNLAT

SEQ ID NO: 143 - protein, 7 aa, synthetic construct
DASNLAT

SEQ ID NO: 144 - protein, 7 aa, synthetic construct
DTSNLET

SEQ ID NO: 145 - protein, 7 aa, synthetic construct
DTSNLVS

SEQ ID NO: 146 - protein, 7 aa, synthetic construct
YASNLES

SEQ ID NO: 147 - protein, 9 aa, synthetic construct
QQWSTYPLT

SEQ ID NO: 148 - protein, 9 aa, synthetic construct
QQWDTYPLT

SEQ ID NO: 149 - protein, 9 aa, synthetic construct
QQWDNYPLT

SEQ ID NO: 150 - protein, 9 aa, synthetic construct
QQWNTYPYT

SEQ ID NO: 151 - protein, 9 aa, synthetic construct
QHSWDIPFT

SEQ ID NO: 152 - protein, 119 aa, synthetic construct
EVQLLESGGGLVQPGGSLRLSCAASGFSVTSYGVHWIRQAPGKGLEWLGVIWSGGSTDYNAAFISRLTISKDNSKNTVYFQMNSLQAEDTAIYYCARAGDYNYDGFAYWGQGTLVTVSS

SEQ ID NO: 153 - protein, 119 aa, synthetic construct
EVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSS

SEQ ID NO: 154 - protein, 119 aa, synthetic construct
EVQLLESGGGLVQPGGSLRLSCAASGFSVTSYGVHWIRQAPGKGLEWLGVIWSGGSTDYNAAFISRLTISKDNSKNTVYFQMNSLRAEDTAVYYCARAGDANYDGFAYWGQGTLVTVSS

SEQ ID NO: 155 - protein, 121 aa, synthetic construct
EVKLVESGGGLVQPGGSLRLSCATSGFTFSDFYMEWVRQPPGKRLEWIAASRNKANDYTTEYSASVKGRFIVSRDTSQSILYLQMNALRAEDTAIYYCARSYYRYDGMDYWGQGTSVTVSS

SEQ ID NO: 156 - protein, 121 aa, synthetic construct
EVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSS

SEQ ID NO: 157 - protein, 121 aa, synthetic construct
DVQLQESGPGLVKPSQTVSLTCTVTGYSITNDNHWWNWIRQVSGSKLEWMGYIDSSGSSDNNPSLKSQISITRDTSKNQLFLQLNSVTIEDIGTYYCARGGGRDYYGMDYWGQGTSVTVSS

SEQ ID NO: 158 - protein, 121 aa, synthetic construct
QVQLQESGPGLVKPSQTLSLTCTVSGYSITNDNHWWNWIRQHPGKGLEWMGYIDSSGSSDNNPSLKSQITISRDTSKNQLSLKLSSVTAADTAVYYCARGGGRDYYGMDYWGQGTTVTVSS

SEQ ID NO: 159 - protein, 106 aa, synthetic construct
DIVLTQSPSSLSASVGDRVTITCSGSSSVSFMYWYQQRPGKAPRLLIYDTSNLASGVPSRFSGSGSGTSYTFTISSLQPEDIATYYCQQWSTYPLTFGQGTKVEVK

SEQ ID NO: 160 - protein, 106 aa, synthetic construct
DIQMTQSPSSLSASVGDRVTITCQASQSVSFLYWYQQRPGKAPRLLIYDTSNLASGVPSRFSGSGSGTSYTFTISSLQPEDIATYYCQQWSTYPLTFGQGTKVEIK

SEQ ID NO: 161 - protein, 106 aa, synthetic construct
DIQMTQSPSSLSASVGDRVTITCQASSSVSFMYWYQQRPGKAPRLLIYDTSNLASGVPSRFSGSGSGTSYTFTISSLQPEDIATYYCQQWSTYPLTFGQGTKVEIK

SEQ ID NO: 162 - protein, 106 aa, synthetic construct
QVVLTQSPVIMSASPGEKVTMTCSAISSVSYMYWYQQKPGSSPRLLIYDTSNLVSGVPVRFSGSGSGTSYSLTISRMEAEDAATYYCQQWNTYPYTFGGGTKLEIK

SEQ ID NO: 163 - protein, 106 aa, synthetic construct
EIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIK

SEQ ID NO: 164 - protein, 111 aa, synthetic construct
DIVLTQSPASLAVSLGQRATISCRASQSVSTSSYSYVHWYQQKPGQPPKLLIKYASNLESGVPARFSGSGSGTDFTLNIHPVEEEDTATYYCQHSWDIPFTFGGGTKLEIK

SEQ ID NO: 165 - protein, 111 aa, synthetic construct
DIVMTQSPDSLGVSLGERATINCRASQSVSTSSYSYVHWYQQKPGQPPKLLIKYASNLESGVPDRFSGSGSGTDFTLTISSLQAEDVAVYYCQHSWDIPFTFGQGTKLEIK

SEQ ID NO: 166 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 167 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - protein, 330 aa, Homo sapiens [sequence omitted]

SEQ ID NO: 169 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 173 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 107 aa, Homo sapiens [sequence omitted]

SEQ ID NO: 177 - protein, 486 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 178 - protein, 486 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 179 - protein, 489 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 489 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 489 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 489 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 485 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 184 - protein, 489 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 489 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 488 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLLESGGGLVQPGGSLRLSCAASGFTVTSYAVHWVRQAPGKGLEWLGVIWSGGSTDYNAAFISRLTISKDNSKNTVYFQMNSLRAEDTAVYYCARAGDANYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 488 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 188 - protein, 488 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 189 - protein, 491 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 491 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 491 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 491 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 487 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 194 - protein, 491 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 491 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 490 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEVQLVESGGGLVQPGGSLRLSCAASGFTFSDFYMEWVRQAPGKGLEWIAASRNKANDYTTEYSASVKGRFIVSRDDSKNSLYLQMNSLKTEDTAVYYCARSYYRYDGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 253 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXDIQMTQSPSSLSASVGDRVTITCQASQSVSFLYWYQQRPGKAPRLLIYDTSNLASGVPSRFSGSGSGTSYTFTISSLQPEDIATYYCQQWSTYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 253 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXDIQMTQSPSSLSASVGDRVTITCQASSSVSFMYWYQQRPGKAPRLLIYDTSNLASGVPSRFSGSGSGTSYTFTISSLQPEDIATYYCQQWSTYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 253 aa, synthetic construct
  VARIANT 1..40: note = Any amino acid
  VARIANT 1..40: note = This region may encompass 14-40 residues
XXXXXXXXXXXXXXXXXXXXXXXXXXXXXXXXXXXXXXXXEIVLTQSPATLSLSPGERATLSCSAISSVSYMYWYQQKPGQAPRLLIYDTSNLVSGVPARFSGSGSGTDYTLTISSLEPEDFAVYYCQQWNTYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 19 aa, synthetic construct
MKLWLNWVFLLTLLHGIQC

SEQ ID NO: 201 - protein, 22 aa, synthetic construct
MDFQVQIFSFLLISASVIVSRG

SEQ ID NO: 202 - protein, 8 aa, synthetic construct
GFTFSDFY

SEQ ID NO: 203 - protein, 10 aa, synthetic construct
GFTFSDFYME

SEQ ID NO: 204 - protein, 7 aa, synthetic construct
GFTFSDF

SEQ ID NO: 205 - protein, 6 aa, synthetic construct
SDFYME

SEQ ID NO: 206 - protein, 8 aa, synthetic construct
GFTVTSYA

SEQ ID NO: 207 - protein, 10 aa, synthetic construct
GFTVTSYAVH

SEQ ID NO: 208 - protein, 7 aa, synthetic construct
GFTVTSY

SEQ ID NO: 209 - protein, 6 aa, synthetic construct
TSYAVH

SEQ ID NO: 210 - protein, 10 aa, synthetic construct
SRNKANDYTT

SEQ ID NO: 211 - protein, 12 aa, synthetic construct
ASRNKANDYTTE

SEQ ID NO: 212 - protein, 8 aa, synthetic construct
RNKANDYT

SEQ ID NO: 213 - protein, 15 aa, synthetic construct
WIAASRNKANDYTTE

SEQ ID NO: 214 - protein, 7 aa, synthetic construct
IWSGGST

SEQ ID NO: 215 - protein, 9 aa, synthetic construct
VIWSGGSTD

SEQ ID NO: 216 - protein, 5 aa, synthetic construct
WSGGS

SEQ ID NO: 217 - protein, 12 aa, synthetic construct
WLGVIWSGGSTD

SEQ ID NO: 218 - protein, 12 aa, synthetic construct
ARSYYRYDGMDY

SEQ ID NO: 219 - protein, 11 aa, synthetic construct
ARSYYRYDGMD

SEQ ID NO: 220 - protein, 13 aa, synthetic construct
ARAGDANYDGFAY

SEQ ID NO: 221 - protein, 12 aa, synthetic construct
ARAGDANYDGFA

SEQ ID NO: 222 - protein, 6 aa, synthetic construct
SYMYWY

SEQ ID NO: 223 - protein, 5 aa, synthetic construct
QSVSF

SEQ ID NO: 224 - protein, 6 aa, synthetic construct
SFLYWY

SEQ ID NO: 225 - protein, 5 aa, synthetic construct
SSVSF

SEQ ID NO: 226 - protein, 6 aa, synthetic construct
SFMYWY

SEQ ID NO: 227 - protein, 10 aa, synthetic construct
LLIYDTSNLV

SEQ ID NO: 228 - protein, 10 aa, synthetic construct
LLIYDTSNLA

SEQ ID NO: 229 - protein, 8 aa, synthetic construct
QQWNTYPY

SEQ ID NO: 230 - protein, 8 aa, synthetic construct
QQWSTYPL

SEQ ID NO: 231 - protein, 5 aa, synthetic construct
SHWIT

SEQ ID NO: 232 - protein, 10 aa, synthetic construct
GYTFTSHWIT

SEQ ID NO: 233 - protein, 7 aa, synthetic construct
GYTFTSH

SEQ ID NO: 234 - protein, 6 aa, synthetic construct
TSHWIT

SEQ ID NO: 235 - protein, 5 aa, synthetic construct
SHWIS

SEQ ID NO: 236 - protein, 10 aa, synthetic construct
GYTFTSHWIS

SEQ ID NO: 237 - protein, 6 aa, synthetic construct
TSHWIS

SEQ ID NO: 238 - protein, 17 aa, synthetic construct
DIYPGSGNTNYNEKFKS

SEQ ID NO: 239 - protein, 10 aa, synthetic construct
DIYPGSGNTN

SEQ ID NO: 240 - protein, 6 aa, synthetic construct
YPGSGN

SEQ ID NO: 241 - protein, 13 aa, synthetic construct
WIGDIYPGSGNTN

SEQ ID NO: 242 - protein, 17 aa, synthetic construct
DIYPGSGNTNYNEKLQG

SEQ ID NO: 243 - protein, 13 aa, synthetic construct
WMGDIYPGSGNTN

SEQ ID NO: 244 - protein, 17 aa, synthetic construct
DIYPGSGNTNYNEKFQG

SEQ ID NO: 245 - protein, 7 aa, synthetic construct
ERGGFDY

SEQ ID NO: 246 - protein, 8 aa, synthetic construct
ARERGGFD

SEQ ID NO: 247 - protein, 11 aa, synthetic construct
RASQDISNFLN

SEQ ID NO: 248 - protein, 7 aa, synthetic construct
SNFLNWY

SEQ ID NO: 249 - protein, 11 aa, synthetic construct
QASQDISNFLN

SEQ ID NO: 250 - protein, 7 aa, synthetic construct
YTSGLHS

SEQ ID NO: 251 - protein, 10 aa, synthetic construct
LLIYYTSGLH

SEQ ID NO: 252 - protein, 7 aa, synthetic construct
YTSGLHT

SEQ ID NO: 253 - protein, 8 aa, synthetic construct
QQDNNHPY